SEQUENCE LISTING (150 sequences)

SEQ ID NO: 1 - DNA, 20 bp, Artificial Sequence
  SYNTHESIZED
aacctgcgaaggacgctgtc

SEQ ID NO: 2 - DNA, 20 bp, Artificial Sequence
  SYNTHESIZED
agagattctgtgcttccacg

SEQ ID NO: 3 - DNA, 20 bp, Artificial Sequence
  SYNTHESIZED
ctgctgctctacgcgccagt

SEQ ID NO: 4 - DNA, 20 bp, Artificial Sequence
  SYNTHESIZED
tgcagtcgggcggtgtgtat

SEQ ID NO: 5 - DNA, 20 bp, Artificial Sequence
  SYNTHESIZED
ccctgtcctgactcgccacc

SEQ ID NO: 6 - DNA, 20 bp, Artificial Sequence
  SYNTHESIZED
gaccgcaacctataccgctg

SEQ ID NO: 7 - DNA, 20 bp, Artificial Sequence
  SYNTHESIZED
cgacgtgacgtttgcagtga

SEQ ID NO: 8 - DNA, 20 bp, Artificial Sequence
  SYNTHESIZED
attattaccccctgctccaa

SEQ ID NO: 9 - DNA, 20 bp, Artificial Sequence
  SYNTHESIZED
catcactgcaaacgtcacgt

SEQ ID NO: 10 - DNA, 20 bp, Artificial Sequence
  SYNTHESIZED
attcatgtgcgattcagatt

SEQ ID NO: 11 - DNA, 20 bp, Artificial Sequence
  SYNTHESIZED
ttagcacagagatcttcaat

SEQ ID NO: 12 - DNA, 20 bp, Artificial Sequence
  SYNTHESIZED
tatcatcgtatagaaaacaa

SEQ ID NO: 13 - DNA, 20 bp, Artificial Sequence
  SYNTHESIZED
ccgcgtcgtagttgttgcct

SEQ ID NO: 14 - DNA, 20 bp, Artificial Sequence
  SYNTHESIZED
cttgatgaagacctcgtcgc

SEQ ID NO: 15 - DNA, 20 bp, Artificial Sequence
  SYNTHESIZED
cacgcgcggcaccgtggtgt

SEQ ID NO: 16 - DNA, 20 bp, Artificial Sequence
  SYNTHESIZED
tggcgattcgaagattcctt

SEQ ID NO: 17 - DNA, 20 bp, Artificial Sequence
  SYNTHESIZED
ggatccatataatgtgttaa

SEQ ID NO: 18 - DNA, 20 bp, Artificial Sequence
  SYNTHESIZED
tctttgtctgctctgcatcg

SEQ ID NO: 19 - DNA, 20 bp, Artificial Sequence
  SYNTHESIZED
cgtgacgccagtgaagaatc

SEQ ID NO: 20 - DNA, 20 bp, Artificial Sequence
  SYNTHESIZED
catgtcaccaaaagcattca

SEQ ID NO: 21 - DNA, 20 bp, Artificial Sequence
  SYNTHESIZED
acaatggccatgaatgcttt

SEQ ID NO: 22 - DNA, 20 bp, Artificial Sequence
  SYNTHESIZED
catgaatctttcgctcgtcc

SEQ ID NO: 23 - DNA, 20 bp, Artificial Sequence
  SYNTHESIZED
acacagaagattatatggcg

SEQ ID NO: 24 - DNA, 20 bp, Artificial Sequence
  SYNTHESIZED
cacagagtcaagcgattaac

SEQ ID NO: 25 - DNA, 20 bp, Artificial Sequence
  SYNTHESIZED
gattgccattcgaaaagccc

SEQ ID NO: 26 - DNA, 20 bp, Artificial Sequence
  SYNTHESIZED
taatgaatatgcttctaagc

SEQ ID NO: 27 - DNA, 20 bp, Artificial Sequence
  SYNTHESIZED
gtcctccctatgattcttat

SEQ ID NO: 28 - DNA, 20 bp, Artificial Sequence
  SYNTHESIZED
tccgaagcccaaatacattg

SEQ ID NO: 29 - DNA, 20 bp, Artificial Sequence
  SYNTHESIZED
catccaccaataagaatcat

SEQ ID NO: 30 - DNA, 20 bp, Artificial Sequence
  SYNTHESIZED
tgcttgtccaagtaaccgac

SEQ ID NO: 31 - DNA, 20 bp, Artificial Sequence
  SYNTHESIZED
aaccaatccgatgcatgtgt

SEQ ID NO: 32 - DNA, 20 bp, Artificial Sequence
  SYNTHESIZED
agctgttgccatgacggccc

SEQ ID NO: 33 - DNA, 20 bp, Artificial Sequence
  SYNTHESIZED
acggccgcctcgctgatgaa

SEQ ID NO: 34 - DNA, 20 bp, Artificial Sequence
  SYNTHESIZED
gacctcggtgtcagcgctgt

SEQ ID NO: 35 - DNA, 20 bp, Artificial Sequence
  SYNTHESIZED
agacgtgcaccatgccgtag

SEQ ID NO: 36 - DNA, 20 bp, Artificial Sequence
  SYNTHESIZED
gtatcatgcagctaaccaca

SEQ ID NO: 37 - DNA, 20 bp, Artificial Sequence
  SYNTHESIZED
agaacacagccagccaatta

SEQ ID NO: 38 - DNA, 20 bp, Artificial Sequence
  SYNTHESIZED
gcgcttacctgggcctccag

SEQ ID NO: 39 - DNA, 20 bp, Artificial Sequence
  SYNTHESIZED
gtccacgccgccacccgtca

SEQ ID NO: 40 - DNA, 20 bp, Artificial Sequence
  SYNTHESIZED
gtgaagtatctgcgcagtca

SEQ ID NO: 41 - DNA, 20 bp, Artificial Sequence
  SYNTHESIZED
aacccaatgagttctagaaa

SEQ ID NO: 42 - DNA, 20 bp, Artificial Sequence
  SYTHESIZED
attatacctcaatatccctc

SEQ ID NO: 43 - DNA, 20 bp, Artificial Sequence
  SYNTHESIZED
ttaccgtaacaggagttctt

SEQ ID NO: 44 - DNA, 20 bp, Artificial Sequence
  SYNTHESIZED
ttcccaaagaactcctgtta

SEQ ID NO: 45 - DNA, 20 bp, Artificial Sequence
  SYNTHESIZED
cacagtcgtcatcgtggagg

SEQ ID NO: 46 - DNA, 20 bp, Artificial Sequence
  SYNTHESIZED
cacctgccatgagcgcagtg

SEQ ID NO: 47 - DNA, 20 bp, Artificial Sequence
  SYNTHESIZED
agagactccacgtcccgctc

SEQ ID NO: 48 - DNA, 20 bp, Artificial Sequence
  SYNTHESIZED
acgtggagaccgctctgcgc

SEQ ID NO: 49 - DNA, 20 bp, Artificial Sequence
  SYNTHESIZED
cactggggaccgctccgggc

SEQ ID NO: 50 - DNA, 20 bp, Artificial Sequence
  SYNTHESIZED
gggcaatgccgcagctttcc

SEQ ID NO: 51 - DNA, 20 bp, Artificial Sequence
  SYNTHESIZED
caaacccactgttcacgcga

SEQ ID NO: 52 - DNA, 20 bp, Artificial Sequence
  SYNTHESIZED′
ccaagtctatcaccaagacg

SEQ ID NO: 53 - DNA, 20 bp, Artificial Sequence
  SYNTHESIZED
ggctgacgtcttccacgtct

SEQ ID NO: 54 - DNA, 20 bp, Artificial Sequence
  SYNTHESIZED
agtcgagagccaacatattc

SEQ ID NO: 55 - DNA, 20 bp, Artificial Sequence
  SYNTHESIZED
tactgacaatgatgtatatg

SEQ ID NO: 56 - DNA, 20 bp, Artificial Sequence
  SYNTHESIZED
agcaactgtcgagattgcag

SEQ ID NO: 57 - DNA, 20 bp, Artificial Sequence
  SYNTHESIZED
atccgcagaggacagtcatc

SEQ ID NO: 58 - DNA, 20 bp, Artificial Sequence
  SYNTHESIZED
ccttccgggcggggatccac

SEQ ID NO: 59 - DNA, 20 bp, Artificial Sequence
  SYNTHESIZED
gctccaacaactcttcagct

SEQ ID NO: 60 - DNA, 20 bp, Artificial Sequence
  SYNTHESIZED
ctgctcttccatgcttgctt

SEQ ID NO: 61 - DNA, 20 bp, Artificial Sequence
  SYNTHESIZED
aaacttaggcgcctcctcca

SEQ ID NO: 62 - DNA, 20 bp, Artificial Sequence
  SYNTHESIZED
tacagcagagacgtcatact

SEQ ID NO: 63 - DNA, 20 bp, Artificial Sequence
  SYNTHESIZED
atgctcatgacgttcgcctc

SEQ ID NO: 64 - DNA, 20 bp, Artificial Sequence
  SYNTHESIZED
gaggacgagcaccataatta

SEQ ID NO: 65 - DNA, 20 bp, Artificial Sequence
  SYNTHESIZED
tgccaagtacctccggccgg

SEQ ID NO: 66 - DNA, 20 bp, Artificial Sequence
  SYNTHESIZED
ggacctctccagcctggtcc

SEQ ID NO: 67 - DNA, 20 bp, Artificial Sequence
  SYNTHESIZED
tggcccaatccccgggaaat

SEQ ID NO: 68 - DNA, 20 bp, Artificial Sequence
  SYNTHESIZED
gcatcatggcgccgtcgctg

SEQ ID NO: 69 - DNA, 20 bp, Artificial Sequence
  SYNTHESIZED
caggcacttacgctgcgcag

SEQ ID NO: 70 - DNA, 20 bp, Artificial Sequence
  SYNTHESIZED
caaggacatttgatacgtta

SEQ ID NO: 71 - DNA, 20 bp, Artificial Sequence
  SYNTHESIZED
ccatttaacatacattcgtg

SEQ ID NO: 72 - DNA, 20 bp, Artificial Sequence
  SYNTHESIZED
gccaatatgaaacttatcag

SEQ ID NO: 73 - DNA, 20 bp, Artificial Sequence
  SYNTHESIZED
cctgtttggtaatcatcatc

SEQ ID NO: 74 - DNA, 20 bp, Artificial Sequence
  SYNTHESIZED
tatccacaacttcacactta

SEQ ID NO: 75 - DNA, 20 bp, Artificial Sequence
  SYNTHESIZED
tagcgccatcctcaccaaca

SEQ ID NO: 76 - DNA, 20 bp, Artificial Sequence
  SYNTHESIZED
ttgagtcgggcttcatgagt

SEQ ID NO: 77 - DNA, 20 bp, Artificial Sequence
  SYNTHESIZED
cgccatcttcgccaacatgc

SEQ ID NO: 78 - DNA, 20 bp, Artificial Sequence
  SYNTHESIZED
gagcgacacgcccagcatgt

SEQ ID NO: 79 - DNA, 20 bp, Artificial Sequence
  SYNTHESIZED
cgtcaacaatcccaagaagc

SEQ ID NO: 80 - DNA, 20 bp, Artificial Sequence
  SYNTHESIZED
tcagtcatagaaccgacact

SEQ ID NO: 81 - DNA, 20 bp, Artificial Sequence
  SYNTHESIZED
cgaatccaatgaacagaaga

SEQ ID NO: 82 - DNA, 20 bp, Artificial Sequence
  SYNTHESIZED
agattccttcttctgttcat

SEQ ID NO: 83 - DNA, 20 bp, Artificial Sequence
  SYNTHESIZED
cgtgaaccggagtctgcagc

SEQ ID NO: 84 - DNA, 20 bp, Artificial Sequence
  SYNTHESIZED
ctccctcccaaccaacgtcc

SEQ ID NO: 85 - DNA, 20 bp, Artificial Sequence
  SYNTHESIZED
gtggcaggccaagtggagcg

SEQ ID NO: 86 - DNA, 20 bp, Artificial Sequence
  SYNTHESIZED
agtccggctcacataggaac

SEQ ID NO: 87 - DNA, 20 bp, Artificial Sequence
  SYNTHEISZED
ctgctgctgctgcttctacc

SEQ ID NO: 88 - DNA, 20 bp, Artificial Sequence
  SYNTHESIZED
ccaggatattacgtgtgcac

SEQ ID NO: 89 - DNA, 20 bp, Artificial Sequence
  SYNTHESIZED
gtgcttcgtgcgccgaactc

SEQ ID NO: 90 - DNA, 20 bp, Artificial Sequence
  SYNTHESIZED
aacggtccccggagcaaacc

SEQ ID NO: 91 - DNA, 20 bp, Artificial Sequence
  SYNTHESIZED
tctgaagccaataaaccatc

SEQ ID NO: 92 - DNA, 20 bp, Artificial Sequence
  SYNTHESIZED
gaagttggacgcgatcatgt

SEQ ID NO: 93 - DNA, 20 bp, Artificial Sequence
  SYNTHESIZED
cggatgcgaccctcccagac

SEQ ID NO: 94 - DNA, 20 bp, Artificial Sequence
  SYNTHESIZED
tgctgaatatgcctgctaca

SEQ ID NO: 95 - DNA, 20 bp, Artificial Sequence
  SYNTHESIZED
tcccggaccccgctgtgttc

SEQ ID NO: 96 - DNA, 20 bp, Artificial Sequence
  SYNTHESIZED
gctttgtcagcaaaaattcc

SEQ ID NO: 97 - DNA, 20 bp, Artificial Sequence
  SYNTHESIZED
taccatccgtcgtaaaacaa

SEQ ID NO: 98 - DNA, 20 bp, Artificial Sequence
  SYNTHESIZED
aatggttgacaacattcatc

SEQ ID NO: 99 - DNA, 20 bp, Artificial Sequence
  SYNTHESIZED
cgatagcagaagaacgcctc

SEQ ID NO: 100 - DNA, 20 bp, Artificial Sequence
  SYNTHESIZED
gcttataatttactaatgac

SEQ ID NO: 101 - DNA, 20 bp, Artificial Sequence
  SYNTHESIZED
acaagtactcaatgacacag

SEQ ID NO: 102 - DNA, 20 bp, Artificial Sequence
  SYNTHESIZED
gccataatgctcattggtac

SEQ ID NO: 103 - DNA, 20 bp, Artificial Sequence
  SYNTHESIZED
ttattacatgacctgcaaat

SEQ ID NO: 104 - DNA, 20 bp, Artificial Sequence
  SYNTHESIZED
tagtggccctgttccagtat

SEQ ID NO: 105 - DNA, 20 bp, Artificial Sequence
  SYNTHESIZED
gtagaatcgccacatccccc

SEQ ID NO: 106 - DNA, 20 bp, Artificial Sequence
  SYNTHESIZED
tccttacctctgccggacag

SEQ ID NO: 107 - DNA, 20 bp, Artificial Sequence
  SYNTHESIZED
ggtgtatgtggtatcgttta

SEQ ID NO: 108 - DNA, 20 bp, Artificial Sequence
  SYNTHESIZED
gtgatgaggttatgttcatg

SEQ ID NO: 109 - DNA, 20 bp, Artificial Sequence
  SYNTHESIZED
ttggtattctcggtctgttt

SEQ ID NO: 110 - DNA, 20 bp, Artificial Sequence
  SYNTHESIZED
agcatatcggtatctccaaa

SEQ ID NO: 111 - DNA, 20 bp, Artificial Sequence
  SYNTHESIZED
gcagaaatgatgtatcaaac

SEQ ID NO: 112 - DNA, 20 bp, Artificial Sequence
  SYNTHESIZED
cttggctttctgtatgcctc

SEQ ID NO: 113 - DNA, 20 bp, Artificial Sequence
  SYNTHESIZED
tcttggcgacgttgccgcgc

SEQ ID NO: 114 - DNA, 20 bp, Artificial Sequence
  SYNTHESIZED
cgaagatggtcgccaagcaa

SEQ ID NO: 115 - DNA, 20 bp, Artificial Sequence
  SYNTHESIZED
tcctacagacgccttctctt

SEQ ID NO: 116 - DNA, 20 bp, Artificial Sequence
  SYNTHESIZED
cctccagtcaatgcgtgccc

SEQ ID NO: 117 - DNA, 20 bp, Artificial Sequence
  SYNTHESIZED
ttaccaatgtagctcccaag

SEQ ID NO: 118 - DNA, 20 bp, Artificial Sequence
  SYNTHESIZED
ggcaacataggcgacatact

SEQ ID NO: 119 - DNA, 20 bp, Artificial Sequence
  SYNTHESIZED
agtccagggcacgcattgac

SEQ ID NO: 120 - DNA, 20 bp, Artificial Sequence
  SYNTHESIZED
cccttcatacctggacaggg

SEQ ID NO: 121 - DNA, 20 bp, Artificial Sequence
  SYNTHESIZED
tggcactgcgcgtcagtagc

SEQ ID NO: 122 - DNA, 20 bp, Artificial Sequence
  SYNTHESIZED
acgtggctgaacagttcggg

SEQ ID NO: 123 - DNA, 20 bp, Artificial Sequence
  SYNTHESIZED
gagaggatgccggcgataga

SEQ ID NO: 124 - DNA, 20 bp, Artificial Sequence
  SYNTHESIZED
ctctgaaccaagttgtccta

SEQ ID NO: 125 - DNA, 20 bp, Artificial Sequence
  SYNTHESIZED
tgtcctgatcctgtcacaag

SEQ ID NO: 126 - DNA, 20 bp, Artificial Sequence
  SYNTHESIZED
acctagagaaagaagtgatc

SEQ ID NO: 127 - DNA, 20 bp, Artificial Sequence
  SYNTHESIZED
tcaaaacaatcttgtcccat

SEQ ID NO: 128 - DNA, 20 bp, Artificial Sequence
  SYNTHESIZED
aaaaatgtagaagatttcac

SEQ ID NO: 129 - DNA, 20 bp, Artificial Sequence
  SYNTHESIZED
ctataacaacactctgttcc

SEQ ID NO: 130 - DNA, 20 bp, Artificial Sequence
  SYNTHESIZED
gaccctagtaagcacatatt

SEQ ID NO: 131 - DNA, 20 bp, Artificial Sequence
  SYNTHESIZED
tctatttggagccagtagac

SEQ ID NO: 132 - DNA, 20 bp, Artificial Sequence
  SYNTHESIZED
cagggttatactggcgaata

SEQ ID NO: 133 - DNA, 20 bp, Artificial Sequence
  SYNTHESIZED
aagcaacaatgaccacgacc

SEQ ID NO: 134 - DNA, 20 bp, Artificial Sequence
  SYNTHESIZED
acagacattccgaatgtcga

SEQ ID NO: 135 - DNA, 20 bp, Artificial Sequence
  SYNTHESIZED
aaggtggtacgtgacgatgg

SEQ ID NO: 136 - DNA, 20 bp, Artificial Sequence
  SYNTHESIZED
ctcggtcatcaaaccagtta

SEQ ID NO: 137 - DNA, 20 bp, Artificial Sequence
  SYNTHESIZED
gtatgccatcctgatgacga

SEQ ID NO: 138 - DNA, 20 bp, Artificial Sequence
  SYNTHESIZED
ccgccatcatcactgccgtg

SEQ ID NO: 139 - DNA, 20 bp, Artificial Sequence
  SYNTHESIZED
ggccagggcaatgttccgca

SEQ ID NO: 140 - DNA, 20 bp, Artificial Sequence
  SYNTHESIZED
tgtttgactctcccgtctct

SEQ ID NO: 141 - DNA, 20 bp, Artificial Sequence
  SYNTHESIZED
ggtgatcacaacttcactct

SEQ ID NO: 142 - DNA, 20 bp, Artificial Sequence
  SYNTHESIZED
tgtcttcatcgccggcatcg

SEQ ID NO: 143 - DNA, 20 bp, Artificial Sequence
  SYNTHESIZED
acacgcaccccagctcgctg

SEQ ID NO: 144 - DNA, 20 bp, Artificial Sequence
  SYNTHESIZED
gcttcgccaacagcagcgac

SEQ ID NO: 145 - DNA, 20 bp, Artificial Sequence
  SYNTHESIZED
ctatgagtcgtccacgcccc

SEQ ID NO: 146 - DNA, 20 bp, Artificial Sequence
  SYNTHESIZED
ctttcgcctaacgtccgacc

SEQ ID NO: 147 - DNA, 20 bp, Artificial Sequence
  SYNTHESIZED
acactacagacctcgcttgt

SEQ ID NO: 148 - DNA, 20 bp, Artificial Sequence
  SYNTHESIZED
aaagtcgaccccaacaagcg

SEQ ID NO: 149 - DNA, 20 bp, Artificial Sequence
  SYNTHESIZED
cgcttccgcggcccgttcaa

SEQ ID NO: 150 - DNA, 20 bp, Artificial Sequence
  SYNTHESIZED
atcgtttccgcttaacggcg

(19) United States
(12) Patent Application Publication
(10) Pub. No.: US 2017/0101642 A1
(43) Pub. Date: Apr. 13, 2017

(54) METHODS OF INHIBITING VIRAL REPLICATION COMPRISING THE SIGNAL PEPTIDASE COMPLEX

(71) Applicant: Washington University, St. Louis, MO (US)

(72) Inventors: Michael Diamond, St. Louis, MO (US); Rong Zhang, St. Louis, MO (US)

(21) Appl. No.: 15/289,856

(22) Filed: Oct. 10, 2016

Related U.S. Application Data
(60) Provisional application No. 62/239,067, filed on Oct. 8, 2015.
(60) Provisional application No. 62/239,455, filed on Oct. 9, 2015.

(51) Int. Cl. C12N 15/113

(52) U.S. Cl. CPC C12N 15/1137; C12N 2310/14

(57) ABSTRACT

The present invention is directed to compositions targeting the signal peptidase complex and methods of use in treating and preventing flavivirus infection.

### CROSS REFERENCE TO RELATED APPLICATIONS

This application claims the benefit of U.S. Provisional Application No. 62/239,067, filed Oct. 8, 2015, and U.S. Provisional Application No. 62/239,455, filed Oct. 9, 2015, each of the disclosures of which are hereby incorporated by reference in its entirety.

### FIELD OF THE INVENTION

The present invention is directed to compositions targeting the signal peptidase complex and methods of use in treating and preventing flavivirus infection.

### BACKGROUND OF THE INVENTION

West Nile virus (WNV) is a mosquito-transmitted flavivirus that infects humans and other vertebrate animals and is closely related to several other pathogens (e.g., Dengue (DENV), Japanese encephalitis (JEV), and yellow fever (YFV) viruses) that cause global disease. Despite almost 400 million flavivirus infections annually, there is no specific antiviral therapy for this group of viruses.

Thus, there is a need in the art for novel antiviral therapies for the treatment of flaviviruses.

### SUMMARY OF THE INVENTION

In an aspect, the disclosure provides a method to inhibit flaviviral infection, the method comprising contacting a cell with a composition comprising a compound that downregulates or inhibits the ER signal peptidase complex components SPCS1, SPCS2 and/or SPCS3.

In another aspect, the disclosure provides a method to prevent flaviviral infection in a subject, the method comprising administering to the subject a composition comprising a compound that downregulates or inhibits the ER signal peptidase complex components SPCS1, SPCS2 and/or SPCS3.

In still another aspect, the disclosure provides a method to reduce the amount of flavivirus in a subject infected with a flavivirus, the method comprising administering to the subject a composition comprising a compound that downregulates or inhibits the ER signal peptidase complex components SPCS1, SPCS2 and/or SPCS3.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D, FIG. 1E, FIG. 1F, FIG. 1G, FIG. 1H, FIG. 1I and FIG. 1J depict graphs and immunoblots showing genes required for flavivirus infection based on gene editing studies. FIG. 1A depicts WNV infection in 293T and FIG. 1B depicts WNV infection in HeLa gene-edited cells. 293T or HeLa cells were transduced with plasmids encoding sgRNA against the indicated genes (two sgRNA per gene), the Cas9 gene, and a selectable drug marker (puromycin). After three days of drug selection, cells were infected with WNV at an MOI of 5, and 12 hours later analyzed for intracellular E protein expression by flow cytometry. The results are the average of three independent experiments that were normalized to the sgRNA control. Error bars indicate standard error of the means (SEM). Statistical significance using an ANOVA with a multiple comparisons correction was as follows: 293 T cells: P<0.05: SEC61B, SERP1, SPSC1, STT3A, OST4, OSTC, EMC3, EMC5, EMC6; P<0.01: SEC63, SPSC3; HeLa cells: P<0.05: SEC61B, SEC63, OSTC, EMC2, EMC4, EMC5, EMC6; P<0.01: SERP1, SPSC1, SPSC3, STT3A. Dashed lines indicate the normalized level of WNV infection in cells transduced with an sgRNA control. FIG. 1C, FIG. 1D, FIG. 1E depict a Western blot to confirm the efficiency of gene editing for three of the genes (SEC61B, SPCS1, and SPCS3) in FIG. 1A. β-actin is included as a loading control. FIG. 1F depicts 293T cells expressing the indicated sgRNA infected with WNV (MOI of 0.01) and FIG. 1G depicts 293T cells expressing the indicated sgRNA infected with JEV (MOI of 0.1). Supernatants were titrated for infectious virus by focus-forming assay. The data is representative of three independent experiments, each performed in triplicate. Error bars indicate SEM. FIG. 1H depicts the effect of gene editing on related flavivirus JEV (MOI of 50); FIG. 1I depicts the effect of gene editing on DENV (MOI of 3); and FIG. 1J depicts the effect of gene editing on YFV (MOI of 3) infection in 293T cells. Cells were transduced with individual sgRNA against the indicated genes as described in FIG. 1A and harvested at 22 (JEV), 32 (DENV), or 38 (YFV) h after infection for processing by flow cytometry. The results are the average of three independent experiments that were normalized to the sgRNA control. Compared to the sgRNA control, for JEV and DENV, all differences were statistically significant using an ANOVA with a multiple corrections correction (P<0.01). Compared to the sgRNA control, for YFV, sgRNA against the following showed statistically significance using an ANOVA with a multiple corrections correction: (P<0.01: SEC61B, SEC63, SSR3, SPCS1, SPCS3, STT3A, OSTC).

FIG. 2A, FIG. 2B, FIG. 2C and FIG. 2D depicts graphs showing the conserved requirement for ER-associated genes in flavivirus infection in insect cells. FIG. 2A depicts Drosophila DL1 cells treated with the indicated dsRNAs and infected with WNV (Kunjin) (MOI, 4) and FIG. 2B depicts Drosophila DL1 cells treated with the indicated dsRNAs and infected with DENV-2 (MOI, 1) for 30 h, then processed for viral antigen staining by automated immunofluorescence microscopy. The percentage of infected cells was determined by automated microscopy and normalized to the control β-galactosidase siRNA. The data is expressed as the mean normalized value±SD. Statistically significant differences (*, P<0.05; **, P<0.01; ***, P<0.001; ****, P<0.0001) when compared to control siRNA (Student's t-test) are indicated. The data is pooled from four independent experiments tested in duplicate. FIG. 2C depicts cell viability analysis. DL1 cells were treated with the indicated dsRNA and 30 h later processed for cell viability. FIG. 2D depicts AAG2 cells treated with the indicated dsRNAs and infected with WNV (Kunjin) (MOI, 4).

FIG. 3A, FIG. 3B, FIG. 3C, FIG. 3D, FIG. 3E, FIG. 3F, FIG. 3G, FIG. 3H, FIG. 3I, FIG. 3J and FIG. 3K depict graphs, immunoblots and a schematic showing validation and mechanism of action of key genes in the flavivirus lifecycle in bulk-edited cells. FIG. 3A depicts individual sgRNA cell lines trans-complemented with cDNA expressing C-terminal FLAG-tagged versions of their respective genes and GFP or an empty vector control and GFP. Transfected cells were sorted by flow cytometry and then infected with WNV at an MOI of 5. Twelve hours later, cells were fixed, permeabilized, stained for intracellular E protein antigen, and processed by flow cytometry. The data is the average of three independent experiments performed in triplicate and reflects the percentage of WNV-infected cells in the fraction of cells expressing GFP. The indicated comparisons were statistically different (****, P<0.0001), as determined by the Mann-Whitney test. FIG. 3B depicts a Western blot of selected trans-complemented genes (e.g., SPCS1 and SPCS3) after incubating with anti-FLAG tag antibody. FIG. 3C, FIG. 3D depict the effect of sgRNA on translation and replication of a WT (FIG. 3C) and NS5 GVD polymerase mutant (FIG. 3D) WNV replicon. A cDNA launched WNV replicon with a minimal CMV promoter (eGFP-NS1-NS5) was transfected into gene-edited 293T cells. At 48 and 72 h after transfection, cells were harvested, and processed for GFP staining by flow cytometry (see FIG. 11). Statistically significant differences are indicated below the WT replicon graph as determined by ANOVA with a multiple comparisons correction. FIG. 3E depicts gene-edited cells were transfected with a WT WNV replicon as described in FIG. 3C, FIG. 3D. At 48 and 72 h after transfection, cells were harvested, stained for surface expression of NS1, and processed by flow cytometry. The data is expressed as the percentage of cells expressing NS1 compared to an isotype control MAb and is gated on GFP⁺ cells. Statistically significant differences were determined by ANOVA with a multiple comparisons correction (**, P<0.01; ****, P<0.0001). FIG. 3F depicts a schematic of the polyprotein processing strategy of flaviviruses¹³. Red and blue arrows indicate sites of cleavage by the host signalase and viral NS3 proteins, respectively. FIG. 3G, FIG. 3H depict immunoblots of control, SPCS1, and SPCS3 gene-edited 293T cells infected with WNV (MOI, 100) or mock-infected. At the indicated time points, lysates were prepared, electrophoresed and Western blotted with (FIG. 3G) anti-E (hE16) or (FIG. 3H) anti-prM-E (CR4293). Under these electrophoresis conditions, natively processed E and prM proteins migrate at ˜50 and 21 kDa, respectively. Higher molecular weight bands (E^{hi} and prM-E^{hi}) that react specifically with the E and prM-E MAbs in infected SPCS1 and SPCS3 gene-edited cells are indicated. FIG. 3I depicts prM-E transfected cells Western blotted with hE16 and FIG. 3J depicts prM-E transfected cells Western blotted with CR4293. Note, the shift of the prM-E bands to high molecular weight in cells with reduced expression of SPCS1 or SPCS3. The results are representative of three independent experiments and a loading control (β-actin) is provided immediately beneath. FIG. 3K depicts 293T cells expressing the indicated sgRNA transfected with a plasmid encoding the prM-E genes. 24 h later, supernatants were harvested and SVPs were quantitated by a capture ELISA. The results are of average several independent experiments performed in triplicate. The asterisks indicate relative SVP levels in the supernatant that are statistically different compared to control cells (****, P<0.001, ANOVA with a Dunnett's multiple comparison test).

FIG. 4A, FIG. 4B, FIG. 4C, FIG. 4D, FIG. 4E and FIG. 4F depict graphs, immunoblots and an images showing the effects of SPCS1 on flavivirus protein processing and infection using a clonal SPCS1^{-/-} gene edited cell line. FIG. 4A depicts Western blotting to confirm the gene editing of SPSCS1 in a clonal (referred to as SPSC1^{-/-}) compared to a control cell line. β-actin is included as a loading control. FIG. 4B depicts SPSC1^{-/-} clonal cells transfected with prM-E or E expression plasmids. In both constructs, the E gene has its native signal sequence (last 25 amino acids of prM) but in the prM-E plasmid, the leader is located as an internal sequence (bottom). 48 hours after transfection, cells were subjected to Western blotting with hE16. Note, the shift of the prM-E bands to high molecular weight in SPSC1^{-/-} cells. The results are representative of independent experiments. FIG. 4C depicts supernatants harvested from prM-E transfected control or of SPSC1^{-/-} clonal cells (or untransfected cells) at 24 and 48 hours and evaluated for levels of SVP using a capture ELISA. The results are the average of two independent experiments performed in triplicate. FIG. 4D depicts WNV infection in control and SPSC1^{-/-} clonal cells at 72 h. Cells were infected at an MOI of 0.01 and analyzed by FFA. FIG. 4E depicts a summary of growth kinetics for WNV and FIG. 4F depicts a summary of growth kinetics for Chikungunya virus.

FIG. 5A, FIG. 5B, FIG. 5C and FIG. 5D depict a schematics and graph of the CRISPR-Cas9 screen for genes required for WNV and JEV infection. (FIG. 5A) Scheme of screen. A pooled lentivirus library containing 122,411 sgRNA (on average, 6 sgRNA per gene) was transduced into 293T-Cas9 cells at an MOI of 0.1. Ten days later, cells were left uninfected or infected with WNV or JEV (MOI of 1 and 3, respectively). After two weeks, surviving cells were harvested and the sgRNA sequences were obtained by next-generation sequencing. These results were compared to uninfected cells to determine sgRNA enrichment. The WNV screen was performed in triplicate on two independent days. The JEV screen was performed in triplicate on a single day. (FIG. 5B) Results of CRISPR-Cas9 screen for host factors required for infection with WNV in 293T cells. (FIG. 5D) Results of CRISPR-Cas9 screen for host factors required for infection with JEV in 293T cells. The x-axis is a list of all genes with sgRNA and the order was generated post-hoc to highlight genes that group together. The y-axis indicates the statistical significance of enrichment of particular genes (as reflected by sequencing of sgRNA) as compared to the uninfected population, and was determined after pooling technical and biological replicates. Filled circles represent genes identified in the virus-selected cell population. Hits were colored if they passed the statistical criteria described in the Supplementary Experimental Procedures. Significant hits were grouped by function and are colored as indicated. (FIG. 5C) Results of gene ontology (GO) enrichment biological process for genes that were enriched in the WNV screen. Enrichment analysis was performed on the 45 candidates using Panther. P values are indicated.

FIG. 6 depicts a graph showing validation of top 45 ‘hits’ from CRISPR-Cas9 screen using individual sgRNA. Lentiviruses co-expressing individual sgRNA (3 to 5 sgRNA per gene), Cas9, and puromycin were transduced into 293T cells. The gene targets were identified as the top chits' as described in the Methods and FIG. 5. After drug selection and recovery, transduced 293T cells were infected with WNV at an MOI of 5. Twelve hours later, cells were analyzed for viral E protein expression using flow cytometry. The data is the average of two independent experiments and is expressed as the percentage of cells that stained positive for WNV E protein.

FIG. 7 depicts a graph showing analysis of cell viability of gene-edited cells. WNV-infected (24 h time point) bulk CRISPR-Cas9 edited cells were evaluated for cell viability using a metabolic MTT (4,5-dimethylthiazol-2-yl)-2,5-diphenyltetrazolium bromide) assay. The results are pooled from several independent experiments performed in duplicate and data was compared to cells edited with a control sgRNA. None of the differences were statistically different compared to the control.

FIG. 8 depicts a graph showing the effect of gene editing on infection by additional RNA viruses. Lentiviruses co-expressing individual sgRNA (4 sgRNA per gene), Cas9, and puromycin were transduced into 293T cells. The 11 gene targets were identified as the top ‘hits’ as described in the text. Cells were infected with alphaviruses (SINV or CHIKV), a bunyavirus (LACV) or a rhabdovirus (VSV). Depending on the virus, cells were harvested at 12 or 24 h after infection and analyzed for intracellular viral antigen staining by flow cytometry using virus-specific monoclonal antibodies. The data is the average of two independent experiments and is expressed as relative infection (viral antigen expression) compared to the sgRNA control.

FIG. 9 depicts flow cytometry plots showing trans-complementation of sgRNA gene-edited cells with FLAG-tagged genes. (Top and middle rows) Individual sgRNA bulk gene-edited cell lines were trans-complemented with cDNA expressing C-terminal FLAG-tagged versions of their respective genes and GFP or an empty vector control and GFP. Transfected cells were analyzed by flow cytometry for expression of the FLAG-tag in the GFP⁺ cells. (Bottom row) Individual sgRNA bulk gene-edited cell lines were trans-complemented with cDNA expressing and empty vector and then stained for the FLAG-tag on the phycoerythrin channel. The data is representative of independent experiments.

FIG. 10 depicts a graph showing silencing of ER-associated SPCS2 in human U2OS cells. Human U2OS cells were transfected with either control of SPCS2 siRNAs and infected with WNV (KUN) (MOI, 1) for 18 h, DENV (MOI, 1) or SINV (MOI, 0.1) and CHIKV (MOI, 2) for 20 h, and processed for automated immunofluorescence microscopy. The percentage of infected cells was determined by automated microscopy and normalized to the control siRNA. The data is expressed as the mean normalized value±SD. Statistically significant differences (*, P<0.05; ***, P<0.001 were compared to control siRNA by a Student's t-test) are indicated. The data is pooled from at least two independent experiments tested in quadruplicate.

FIG. 11 depicts flow cytometric analysis of GFP expression in WNV replicon transfected gene-edited cells. Gene-edited bulk-selected 293T cells (sgControl, sgSTT3A, sgSPCS1, and sgSPCS3) were transfected with a cDNA launched replicon (WT or NS5 GVD polymerase dead mutant) containing a minimal CMV promoter, GFP, and the NS1 through NS5 genes of WNV. At 72 h after infection, cells were processed for GFP expression by flow cytometry. The transfection efficiency was approximately 15 to 30% (% positive cells) and the mean fluorescence intensity (MFI) of the GFP⁺ cells is indicated. In all cells tested, the GVD polymerase dead mutant expresses low levels of GFP, which reflects translation of the replicon RNA generated by the host nuclear DNA-dependent RNA polymerase. In sgControl, sgSPCS1, and sgSPCS3 (but not sgSTT3A) gene-edited cells, the WT replicon supports high levels of GFP expression, which reflects the replication activity of the NS5 RNA-dependent RNA polymerase. The results are representative of at least two independent experiments performed in triplicate, and are shown as contour plots.

FIG. 12 depicts flow cytometric analysis of surface NS1 expression in WNV replicon transfected gene-edited cells. Gene-edited bulk-selected 293T cells (sgControl, sgSTT3A, sgSPCS1, and sgSPCS3) were transfected with a cDNA launched WNV replicon (WT or NS5 GVD polymerase dead mutant) as described in FIG. 9. At 48 h after infection, cells were stained with a biotinylated anti-NS1 (9-NS1) or anti-CHIKV (CHK-152) directly to detect plasma membrane associated NS1 on the cell surface. Cells were processed by two-color flow cytometry and analyzed. The results are representative of at least two independent experiments performed in triplicate.

FIG. 13 depicts an immunoblot showing aberrant processing of WNV E protein in SPCS1 and SPCS3 gene-edited 293T cells. Note, this is an over-exposed Western blot of FIG. 3F, and is shown to highlight the accumulation of high molecular weight bands that react with anti-E protein antibody specifically in SPCS1 and SPCS3 gene-edited 293T cells. Control, SPCS1, and SPCS3 gene-edited 293T cells were infected with WNV (MOI, 100) or mock-infected for the indicated times. Lysates were prepared, boiled in SDS sample buffer, electrophoresed and Western blotted with an anti-E (hE16) MAb. Under these electrophoresis conditions, natively processed E protein migrates at ˜50 to 55 kDa, respectively. Higher molecular weight bands (E^{med} and E^{hi}) that react specifically with the E MAb in infected SPCS1 and SPCS3 gene-edited cells are present only in SPCS1 and SPCS3 gene-edited 293T cells. The data is representative of two independent experiments.

FIG. 14 depicts an immunoblot showing aberrant processing of WNV NS1 protein in SPCS1 and SPCS3 gene-edited 293T cells. Control, SPCS1, and SPCS3 gene-edited 293T cells were infected with WNV (New York 1999, MOI of 100) or mock-infected for the indicated times. Lysates were prepared, boiled in SDS sample buffer in the presence of 5% β-mercaptoethanol, electrophoresed and Western blotted with 8-NS1, a MAb that detects a linear epitope on WNV NS1¹⁴. The gel was separated into two parts (space indicated) due to the much higher signal of NS1 (48 kDa) and NS1′ (53 kDa) in the sgControl cells; thus, the top and bottom are not exposed equally. NS1′ is a C-terminal extended product of NS1 and is generated as the result of a −1 programmed ribosomal frameshift¹⁵. Note the higher molecular weight NS1 species (NS1^{hi}) is present only in SPCS1 and SPCS3 gene-edited cells despite the lower overall levels of NS1. Below, is a Western blot for β-actin to confirm equal loading of lysates. The results are representative of two independent experiments.

FIG. 15A and FIG. 15B depict flow cytometry plots, a graph and an immunoblot showing expression of NS1 and MHC class I in gene-edited cells. (FIG. 15A) The indicated bulk CRISPR-Cas9 edited cells were stained for surface expression of HLA-A2 class I MHC molecules using a specific mAb (W6/32) or an isotype control mAb and flow cytometry. The histograms shown are representative of two independent experiments performed in duplicate. (FIG. 15B) A summary is shown on the left of the normalized mean fluorescence intensity compared to the WT controls. A plasmid encoding WNV NS1 with a host-derived signal sequence (human CD33) was transfected into the indicated bulk CRISPR-Ca9 edited cells and 24 h later lysates were analyzed by Western blotting for NS1 (8-NS1 MAb). The results are representative of two independent experiments.

FIG. 16 depicts flow cytometry plots showing expression of complement regulators and MHC class I on the surface of SPSC1^{-/-} clonal cells. Control and SPSC1^{-/-} cells were stained for surface expression of CD55 (Decay accelerating factor (DAF), GPI-anchored), CD59 (transmembrane), HLA-A2 class I MHC molecules (transmembrane), or CD46 (Membrane cofactor protein, MCP, transmembrane) with specific primary MAbs (red and blue histograms) or isotype control MAbs (green and orange histograms) and processed flow cytometry. The histograms shown are representative of independent experiments performed in duplicate. No differences in cell surface expression between control and SPSC1^{-/-} cells were observed.

FIG. 17 depicts a graph showing the effect of trans-complementation of SPCS1 on WNV infection. Trans-complementation of SPCS1 restores production of WNV.

FIG. 18A, FIG. 18B and FIG. 18C depict graphs shows that the loss of expression of SPCS1 results in very little infectious flavivirus production. Virtually no infectious DENV (FIG. 18A), JEV (FIG. 18B), YFV (FIG. 18C) was recovered over time.

### DETAILED DESCRIPTION OF THE INVENTION

Prior drug development efforts have been focused on defining small molecules that target flavivirus proteins including the viral protease and polymerase. Such molecules exert a rapid selective pressure that generally results in emergence of resistance due to the error prone activity of the RNA-dependent RNA polymerase. In contrast, the inventors sought out to identify host genes required for a key and conserved stage in the viral lifecycle such that inhibition of these host genes could abort flaviviral infection. Several identified genes were associated with endoplasmic reticulum (ER) functions including regulation of translocation, protein degradation, and N-linked glycosylation. Among the genes identified by the inventors, the host signal peptidase genes SPCS1 and SPCS3 were the most prominent. Reduced expression of these genes resulted in markedly lower replication of West Nile, Dengue, Japanese encephalitis, and yellow fever viruses. Remarkably, other unrelated viruses were not affected and the host cell did not show toxicity or cell injury. Accordingly, disclosed herein are compositions and methods for treating and/or preventing flaviviral infection comprising targeting ER functions, specifically, the signal peptidase complex.

Various aspects of the invention are described in more detail below.

### I. Compositions

In an aspect, a composition of the invention comprises a compound that modulates ER-associated functions required for optimal flavivirus translation, polyprotein processing and replication. ER-associated functions include carbohydrate modification, translocation and ERAD. In certain embodiments, a gene involved in ER-associated translocation is selected from the group consisting of SEC63, SEC61B, SRP72, SSR1, SSR3, SPCS1, SPCS2 and SPCS3. In other embodiments, a gene involved in ER-associated carbohydrate modification is selected from the group consisting of OST4, SERP1, STT3A and OSTC. In still other embodiments, a gene involved in ER-associated protein degradation (ERAD) is selected from the group consisting of SEL1L, EMC2, EMC3 and EM6. In an embodiment, a composition of the invention comprises a compound that modulates a gene selected from the group consisting of EMC3, EMC4, EMC6, SEL1L, SEC61B, SEC63, STT3A, OSTC, SERP1, SSR3, SPCS1, and SPCS2. In another embodiment, a composition of the invention comprises a compound that modulates a gene selected from the group consisting of SEC61B, SPCS1 and SPCS3. In still another embodiment, a composition of the invention comprises a compound that modulates a gene selected from the group consisting of STT3A, SEC63, SPSC1 and SPCS3. In an embodiment, a composition of the invention comprises a compound that modulates the ER signal peptidase complex. In a specific embodiment, a composition of the invention comprises a compound that modulates the ER signal peptidase complex components SPCS1, SPCS2 and/or SPCS3. A compound that modulates ER-associated functions may be a compound that downregulates genes involved in ER-associated functions. Specifically, a compound that modulates the ER signal peptidase complex may be a compound that downregulates or inhibits SPCS1, SPCS2 and/or SPCS3. Methods to determine if a compound modulates SPCS1, SPCS2 and/or SPCS3 are known in the art. For example, SPCS1, SPCS2 and/or SPCS3 nucleic acid expression, SPCS1, SPCS2 and/or SPCS3 protein expression, or SPCS1, SPCS2 and/or SPCS3 activity may be measured as described in more detail below.

The signal peptidase complex (SPC) is a protein complex that is located in the endoplasmic reticulum membrane and cleaves the signal sequence from precursor proteins following their transport out of the cytoplasmic space. The SPC comprises signal peptidase complex subunit 1 (SPCS1, also referred to as SPC12, HSPC033, microsomal signal peptidase 12 kDa subunit and SPase 12 kDa subunit), signal peptidase complex subunit 2 (SPCS2, also referred to as SPC25, KIAA0102, microsomal signal peptidase 25 kDa subunit and SPase 25 kDa subunit) and signal peptidase complex subunit 3 (SPCS3, also referred to as SPC22, UNQ1841/PRO3567, microsomal signal peptidase 22/23 kDa subunit, SPC22/23 and SPase 22/23 kDa subunit). The SPC is a key host signalase required for efficient processing of the flavivirus polyprotein. Specifically, components of the SPC are required for proper processing of the viral prM, E and NS1 proteins.

A compound with the ability to modulate an ER-associated function in cells may potentially be used as an antiviral agent. Specifically, a compound with the ability to modulate the SPC in cells may potentially be used as an antiviral agent. Even more specifically, a compound with the ability to modulate SPCS1, SPCS2 and/or SPCS3 in cells may potentially be used as an antiviral agent. A compound with the ability to modulate SPCS1, SPCS2 and/or SPCS3 may include, without limitation, a compound, a drug, a small molecule, a peptide, a nucleic acid molecule, a protein, an antibody, a lipid, a carbohydrate, a sugar, a lipoprotein and combinations thereof. A nucleic acid molecule may be an antisense oligonucleotide, a small interfering RNA (siRNA), a ribozyme, a small nuclear RNA (snRNA), a long noncoding RNA (LncRNA), or a nucleic acid molecule which forms triple helical structures. Such compounds can be isolated from nature (e.g., isolated from organisms) or they can be produced in a laboratory (e.g., recombinantly or synthetically). Also encompassed are compounds that are combinations of natural and synthetic molecules. Methods to isolate or produce recombinant or synthetic candidate compounds are known to those skilled in the art. In certain embodiments, a compound that downregulates or inhibits SPCS1, SPCS2 and/or SPCS3 blocks enzymatic activity of SPCS1, SPCS2 and/or SPCS3. In other embodiments, a compound that downregulates or inhibits SPCS1, SPCS2 and/or SPCS3 reduces SPCS1, SPCS2 and/or SPCS3 protein expression. In still other embodiments, a compound that downregulates or inhibits SPCS1, SPCS2 and/or SPCS3 reduces SPCS1, SPCS2 and/or SPCS3 nucleic acid expression.

i. Nucleic Acid Expression

In an embodiment, SPCS1, SPCS2 and/or SPCS3 nucleic acid expression may be measured to identify a compound that downregulates or inhibits SPCS1, SPCS2 and/or SPCS3. For example, when SPCS1, SPCS2 and/or SPCS3 nucleic acid expression is decreased in the presence of a compound relative to an untreated control, the compound decreases the expression of SPCS1, SPCS2 and/or SPCS3. In a specific embodiment, SPCS1, SPCS2 and/or SPCS3 mRNA may be measured to identify a compound that decreases the expression of SPCS1, SPCS2 and/or SPCS3.

Methods for assessing an amount of nucleic acid expression in cells are well known in the art, and all suitable methods for assessing an amount of nucleic acid expression known to one of skill in the art are contemplated within the scope of the invention. The term “amount of nucleic acid expression” or “level of nucleic acid expression” as used herein refers to a measurable level of expression of the nucleic acids, such as, without limitation, the level of messenger RNA (mRNA) transcript expressed or a specific variant or other portion of the mRNA, the enzymatic or other activities of the nucleic acids, and the level of a specific metabolite. The term “nucleic acid” includes DNA and RNA and can be either double stranded or single stranded. Non-limiting examples of suitable methods to assess an amount of nucleic acid expression may include arrays, such as microarrays, PCR, such as RT-PCR (including quantitative RT-PCR), nuclease protection assays and Northern blot analyses. In a specific embodiment, determining the amount of expression of a target nucleic acid comprises, in part, measuring the level of target nucleic acid mRNA expression.

In one embodiment, the amount of nucleic acid expression may be determined by using an array, such as a microarray. Methods of using a nucleic acid microarray are well and widely known in the art. For example, a nucleic acid probe that is complementary or hybridizable to an expression product of a target gene may be used in the array. The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. The term “probe” as used herein refers to a nucleic acid sequence that will hybridize to a nucleic acid target sequence. In one example, the probe hybridizes to an RNA product of the nucleic acid or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the amount of nucleic acid expression may be determined using PCR. Methods of PCR are well and widely known in the art, and may include quantitative PCR, semi-quantitative PCR, multiplex PCR, or any combination thereof. Specifically, the amount of nucleic acid expression may be determined using quantitative RT-PCR. Methods of performing quantitative RT-PCR are common in the art. In such an embodiment, the primers used for quantitative RT-PCR may comprise a forward and reverse primer for a target gene. The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

The amount of nucleic acid expression may be measured by measuring an entire mRNA transcript for a nucleic acid sequence, or measuring a portion of the mRNA transcript for a nucleic acid sequence. For instance, if a nucleic acid array is utilized to measure the amount of mRNA expression, the array may comprise a probe for a portion of the mRNA of the nucleic acid sequence of interest, or the array may comprise a probe for the full mRNA of the nucleic acid sequence of interest. Similarly, in a PCR reaction, the primers may be designed to amplify the entire cDNA sequence of the nucleic acid sequence of interest, or a portion of the cDNA sequence. One of skill in the art will recognize that there is more than one set of primers that may be used to amplify either the entire cDNA or a portion of the cDNA for a nucleic acid sequence of interest. Methods of designing primers are known in the art. Methods of extracting RNA from a biological sample are known in the art.

The level of expression may or may not be normalized to the level of a control nucleic acid. This allows comparisons between assays that are performed on different occasions.

SPCS1, SPCS2 and/or SPCS3 nucleic acid expression may be increased or decreased in the presence of a compound relative to an untreated control. In one embodiment, SPCS1, SPCS2 and/or SPCS3 nucleic acid expression can be compared using the ratio of the level of expression of SPCS1, SPCS2 and/or SPCS3 nucleic acid in the presence of a compound as compared with the expression level of SPCS1, SPCS2 and/or SPCS3 nucleic acid in the absence of a compound. For example, a nucleic acid is differentially expressed if the ratio of the level of expression of SPCS1, SPCS2 and/or SPCS3 nucleic acid in the presence of a compound as compared with the expression level of SPCS1, SPCS2 and/or SPCS3 nucleic acid in the absence of a compound is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment, the increase or decrease in expression is measured using p-value. For instance, when using p-value, a nucleic acid is identified as being differentially expressed between a SPCS1, SPCS2 and/or SPCS3 nucleic acid in the presence of a compound and SPCS1, SPCS2 and/or SPCS3 nucleic acid in the absence of a compound when the p-value is less than 0.1, preferably less than 0.05, more preferably less than 0.01, even more preferably less than 0.005, the most preferably less than 0.001.

ii. Protein Expression

In another embodiment, SPCS1, SPCS2 and/or SPCS3 protein expression may be measured to identify a compound that downregulates or inhibits the expression of SPCS1, SPCS2 and/or SPCS3. For example, when SPCS1, SPCS2 and/or SPCS3 protein expression is decreased in the presence of a compound relative to an untreated control, the compound decreases the expression of SPCS1, SPCS2 and/or SPCS3. In a specific embodiment, SPCS1, SPCS2 and/or SPCS3 protein expression may be measured using immunoblot.

Methods for assessing an amount of protein expression are well known in the art, and all suitable methods for assessing an amount of protein expression known to one of skill in the art are contemplated within the scope of the invention. Non-limiting examples of suitable methods to assess an amount of protein expression may include epitope binding agent-based methods and mass spectrometry based methods.

In some embodiments, the method to assess an amount of protein expression is mass spectrometry. By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can use mass spectrometry to look for the level of protein encoded from a target nucleic acid of the invention.

In some embodiments, the method to assess an amount of protein expression is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to a target gene protein. Nucleic acids may include RNA, DNA, and naturally occurring or synthetically created derivative.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

In general, an epitope binding agent-based method of assessing an amount of protein expression comprises contacting a sample comprising a polypeptide with an epitope binding agent specific for the polypeptide under conditions effective to allow for formation of a complex between the epitope binding agent and the polypeptide. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, an epitope binding agent-based method is an immunoassay. Immunoassays can be run in a number of different formats. Generally speaking, immunoassays can be divided into two categories: competitive immunoassays and non-competitive immunoassays. In a competitive immunoassay, an unlabeled analyte in a sample competes with labeled analyte to bind an antibody. Unbound analyte is washed away and the bound analyte is measured. In a non-competitive immunoassay, the antibody is labeled, not the analyte. Non-competitive immunoassays may use one antibody (e.g. the capture antibody is labeled) or more than one antibody (e.g. at least one capture antibody which is unlabeled and at least one “capping” or detection antibody which is labeled.) Suitable labels are described above.

In some embodiments, the epitope binding agent-based method is an ELISA. In other embodiments, the epitope binding agent-based method is a radioimmunoassay. In still other embodiments, the epitope binding agent-based method is an immunoblot or Western blot. In alternative embodiments, the epitope binding agent-based method is an array. In another embodiment, the epitope binding agent-based method is flow cytometry. In different embodiments, the epitope binding agent-based method is immunohistochemistry (IHC). IHC uses an antibody to detect and quantify antigens in intact tissue samples. The tissue samples may be fresh-frozen and/or formalin-fixed, paraffin-embedded (or plastic-embedded) tissue blocks prepared for study by IHC. Methods of preparing tissue block for study by IHC, as well as methods of performing IHC are well known in the art.

SPCS1, SPCS2 and/or SPCS3 protein expression may be increased or decreased in the presence of a compound relative to an untreated control. In one embodiment, SPCS1, SPCS2 and/or SPCS3 protein expression can be compared using the ratio of the level of expression of SPCS1, SPCS2 and/or SPCS3 protein in the presence of a compound as compared with the expression level of SPCS1, SPCS2 and/or SPCS3 protein in the absence of a compound. For example, a protein is differentially expressed if the ratio of the level of expression of SPCS1, SPCS2 and/or SPCS3 protein in the presence of a compound as compared with the expression level of SPCS1, SPCS2 and/or SPCS3 protein in the absence of a compound is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment, the increase or decrease in expression is measured using p-value. For instance, when using p-value, a protein is identified as being differentially expressed between SPCS1, SPCS2 and/or SPCS3 protein in the presence of a compound and SPCS1, SPCS2 and/or SPCS3 protein in the absence of a compound when the p-value is less than 0.1, preferably less than 0.05, more preferably less than 0.01, even more preferably less than 0.005, the most preferably less than 0.001.

iii. Activity

In an embodiment, SPCS1, SPCS2 and/or SPCS3 activity may be measured to identify a compound that downregulates or inhibits SPCS1, SPCS2 and/or SPCS3. For example, processing of viral prM, E and NS1 proteins may be measured. In an embodiment, a compound that downregulates or inhibits SPCS1, SPCS2 and/or SPCS3 may reduce the amount of E protein present during viral infection and/or increase the molecular weight of E protein detected following viral infection. In another embodiment, a compound that downregulates or inhibits SPCS1, SPCS2 and/or SPCS3 may reduce the amount of prM-E protein present during viral infection and/or increase the molecular weight of prM-E protein detected following viral infection. In still another embodiment, a compound that downregulates or inhibits SPCS1, SPCS2 and/or SPCS3 may reduce the amount of NS1 protein present during viral infection and/or increase the molecular weight of NS1 protein detected following viral infection. In a different embodiment, a compound that downregulates or inhibits SPCS1, SPCS2 and/or SPCS3 may reduce the level of secreted viral particles (SVPs) following viral infection.

### (a) Components of the Composition

The present disclosure also provides pharmaceutical compositions. The pharmaceutical composition comprises a compound that modulates ER-associated functions, as an active ingredient(s), and at least one pharmaceutically acceptable excipient, carrier or diluent. Further, a composition of the invention may contain binders, fillers, pH modifying agents, disintegrants, dispersants, lubricants, taste-masking agents, flavoring agents, preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition a compound that modulates ER-associated functions is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery a compound that modulates ER-associated functions in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a compound that modulates ER-associated functions may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a compound that modulates ER-associated functions (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A compound that modulates ER-associated functions may be encapsulated in a microemulsion by any method generally known in the art.

In yet another embodiment, a compound that modulates ER-associated functions may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Methods

In an aspect, the present invention encompasses a method to inhibit flaviviral infection. The method comprises contacting a cell with a composition comprising a compound that modulates ER-associated functions required for optimal flavivirus translation, polyprotein processing and replication. In an embodiment, the composition comprises a compound that downregulates or inhibits the ER signal peptidase complex. In another embodiment, the composition comprises a compound that downregulates or inhibits the ER signal peptidase complex components SPCS1, SPCS2 and/or SPCS3. In a specific embodiment, the composition comprises a compound that downregulates or inhibits SPCS1. In another specific embodiment, the flaviviral infection is due to a flavivirus selected from the group consisting of West Nile virus, Dengue virus, Japanese encephalitis virus or yellow fever virus. Since a composition of the present invention is useful for inhibiting infection by a flavivirus, a composition of the invention may be used to protect a subject from flaviviral infection. As used herein, the term “protect” refers to prophylactic as well as therapeutic use. Thus, one embodiment of the present invention is a method to prevent flaviviral infection in a subject by administering a composition comprising a compound that downregulates or inhibits the ER signal peptidase complex components SPCS1, SPCS2 and/or SPCS3.

In another aspect, the present invention encompasses a method to reduce the amount of flavivirus in a subject infected with a flavivirus. The method comprises administering a composition comprising a compound that modulates ER-associated functions required for optimal flavivirus translation, polyprotein processing and replication. In an embodiment, the composition comprises a compound that downregulates or inhibits the ER signal peptidase complex. In another embodiment, the composition comprises a compound that downregulates or inhibits the ER signal peptidase complex components SPCS1, SPCS2 and/or SPCS3. In a specific embodiment, the composition comprises a compound that downregulates or inhibits SPCS1. In another specific embodiment, the flaviviral infection is due to a flavivirus selected from the group consisting of West Nile virus, Dengue virus, Japanese encephalitis virus or yellow fever virus.

In still another aspect, the present invention encompasses a method to protect a subject from flavivirus infection. The method comprises administering to the subject a composition comprising a compound that modulates ER-associated functions required for optimal flavivirus translation, polyprotein processing and replication. In an embodiment, the composition comprises a compound that downregulates or inhibits the ER signal peptidase complex. In another embodiment, the composition comprises a compound that downregulates or inhibits the ER signal peptidase complex components SPCS1, SPCS2 and/or SPCS3. In a specific embodiment, the composition comprises a compound that downregulates or inhibits SPCS1. In another specific embodiment, the flaviviral infection is due to a flavivirus selected from the group consisting of West Nile virus, Dengue virus, Japanese encephalitis virus or yellow fever virus.

As used herein, the terms “viral infection”, “viral infectivity”, “infection by a virus”, “viral propagation”, and the like, refer to the ability of a virus to carry out all steps in the viral life cycle, resulting in the production of infectious particles. Such a life cycle comprises a variety of steps including, for example, attachment, uncoating, transcription, translation, protein processing, replication of nucleic acid molecules, assembly of viral particles, intracellular transport of viral particles, budding, release and the like. Other steps may also be included depending on the virus.

As used herein, the terms “inhibit viral infection”, “inhibit infection by a virus”, “inhibit viral infectivity”, “inhibit viral propagation”, and the like, refer to decreasing the amount of virus present in an infected cell or subject relative to the amount of virus present in a cell or subject that has not been contacted with or treated with the disclosed methods or compounds. Also encompassed is the ability to prevent viral infection. Inhibition of viral infection can be effected in a patient infected with a flavivirus, or it can be effected in cells in culture (e.g., tissue culture). It should be appreciated that the terms amount and concentration can be used interchangeably. An amount of virus can also be referred to as a titer. It is also understood by those of skill in the art that the amount of virus can refer to the total number of viral particles, or it can refer to the number of viral particles that are infectious, i.e. capable of carrying out the viral life cycle, including the ability to effect another cycle of infectious particle formation. For example, in a given population of virus particles, some or all of the particles may be unable to carry out a specific step in its life cycle (e.g., attachment or entry) due to a deficiency in a molecule needed to perform that step. While the number of particles in the population may be large, the number of infectious particles could be small to none. Thus the amount of virus determined by counting virus particles may differ from that determined by measuring functional virus in, for example, a plaque assay. Accordingly methods of the present invention can affect the total number of viral particles produced, as well as the number of infectious viral particles produced. Appropriate methods of determining the amount of virus are understood by those skilled in the art and include, but are not limited to, directly counting virus particles, titering virus in cell culture e.g., plaque assay), measuring the amount of viral protein(s), measuring the amount of viral nucleic acids, or measuring the amount of a reporter protein, e.g., luciferase, GFP.

Inhibition of viral infection can result in a partial reduction in the amount of virus, or it can result in complete elimination of virus from a cell or subject or in prevention of viral infection. In one embodiment of the present invention, the amount of virus is reduced by at least 5%, at least 10%, at least 20%, at least 30%, at least 40%, at least 50%, at least 60%, at least 70%, at least 80%, at least 90%, or at least 95%. In another embodiment, the amount of virus is reduced by a factor of at least 10, at least 50, at least 100, at least 500, at least 1000, at least 5000, or at least 10,000. In one embodiment the viral infection is completely inhibited (i.e., there are no infectious particles).

As used herein, the term “contacting” refers to bringing the compound and the cell into proximity so that the compound is capable of interacting with a gene involved in ER-associated function, or more specifically, SPCS1, SPCS2 and/or SPCS3. Such contacting can be achieved by introducing the compound to the cell when the cell is in a tissue culture environment, or it can be achieved when the cell is present in a subject. Consequently contacting the compound with the infected cell can be achieved through introducing the compound into a subject, for example, through an oral medication, an injection or other route of administration. The compound can interact with and remain on outside of the cell, or it can enter the cell and interact with a gene involved in ER-associated function, or more specifically, SPCS1, SPCS2 and/or SPCS3 within the cell.

The composition is described in Section I, the subject and administration are described in more detail below.

### (a) Subject

A method of the invention may be used to treat or prevent flaviviral infection in a subject that is a human, a livestock animal, a companion animal, a lab animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In certain embodiments, the animal is a laboratory animal. Non-limiting examples of a laboratory animal may include rodents, canines, felines, and non-human primates. In other embodiments, the animal is a rodent. Non-limiting examples of rodents may include mice, rats, guinea pigs, etc. In a specific embodiment, the subject is a human.

Given that many flaviviruses are arthropod-transmitted, in some embodiments, a subject may be an arthropod. Arthropods include insects, arachnids, myriapods, and crustaceans. In an embodiment, the arthropod is an insect. In a specific embodiment, the insect is a mosquito. In an exemplary embodiment, the insect is Drosophila.

### (b) Administration

In certain aspects, a therapeutically effective amount of a composition of the invention may be administered to a subject. Administration is performed using standard effective techniques, including peripherally (i.e. not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. The route of administration may be dictated by the disease or condition to be treated. It is within the skill of one in the art, to determine the route of administration based on the disease or condition to be treated.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the peptides useful in this discovery, making them more lipophilic, for example, by encapsulating them in liposomes or by blocking polar groups.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

For therapeutic applications, a therapeutically effective amount of a composition of the invention is administered to a subject. A “therapeutically effective amount” is an amount of the therapeutic composition sufficient to produce a measurable response (e.g., a reduction in infection, reduction in viral particles, reduction in symptoms associated with viral infection). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, the flavivirus, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The timing of administration of the treatment relative to the disease itself and duration of treatment will be determined by the circumstances surrounding the case. Treatment could begin in a hospital or clinic itself, or at a later time after discharge from the hospital or after being seen in an outpatient clinic.

Duration of treatment could range from a single dose administered on a one-time basis to a life-long course of therapeutic treatments. The duration of treatment can and will vary depending on the subject and the disease or disorder to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The frequency of dosing may be once, twice, three times or more daily or once, twice, three times or more per week or per month, or as needed as to effectively treat the symptoms or disease. In certain embodiments, the frequency of dosing may be once, twice or three times daily. For example, a dose may be administered every 24 hours, every 12 hours, or every 8 hours. In other embodiments, the frequency of dosing may be once, twice or three times weekly. For example, a dose may be administered every 2 days, every 3 days or every 4 days. In a different embodiment, the frequency of dosing may be one, twice, three or four times monthly. For example, a dose may be administered every 1 week, every 2 weeks, every 3 weeks or every 4 weeks.

A compound of the present invention, or a composition thereof, may be administered alone or in combination with one or more other pharmaceutical agents, including other compounds of the present invention.

Although the foregoing methods appear the most convenient and most appropriate and effective for administration of a composition of the invention, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. CRISPR/Cas9 Screen Identifies an Endoplasmic Reticulum-Associated Signal Peptidase Complex Required for Infectivity of Multiple Flaviviruses

West Nile virus (WNV) is a mosquito-transmitted flavivirus that infects humans and other vertebrate animals and is closely related to several other pathogens (e.g., Dengue (DENV), Japanese encephalitis (JEV), and yellow fever (YFV) viruses) that cause global disease¹. Despite almost 400 million flavivirus infections annually, there is no specific antiviral therapy for this group of viruses. We reasoned that an improved understanding of the host factors required for efficient infection might identify genes that could be targeted pharmacologically to control infection of multiple members of the viral genus. Although genome-wide siRNA screens have been performed with WNV and other flaviviruses in different laboratories²⁻⁴, the results have varied.

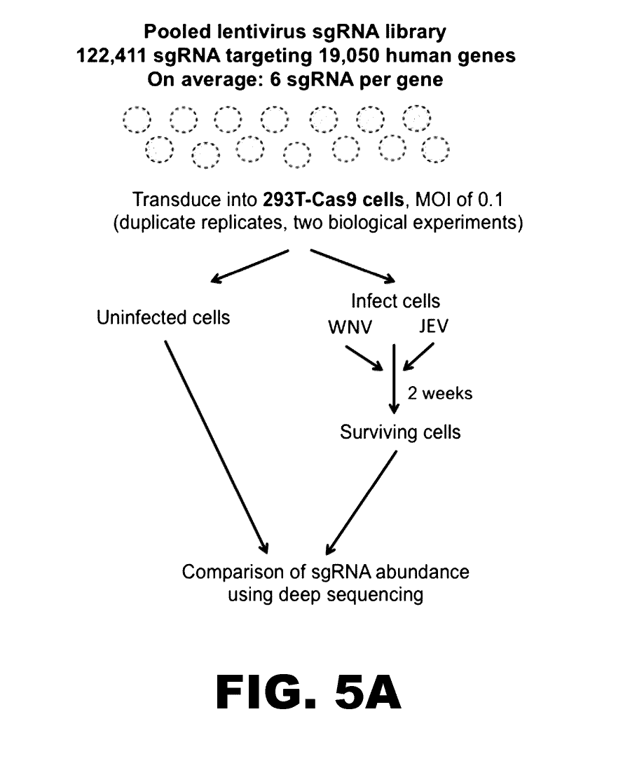

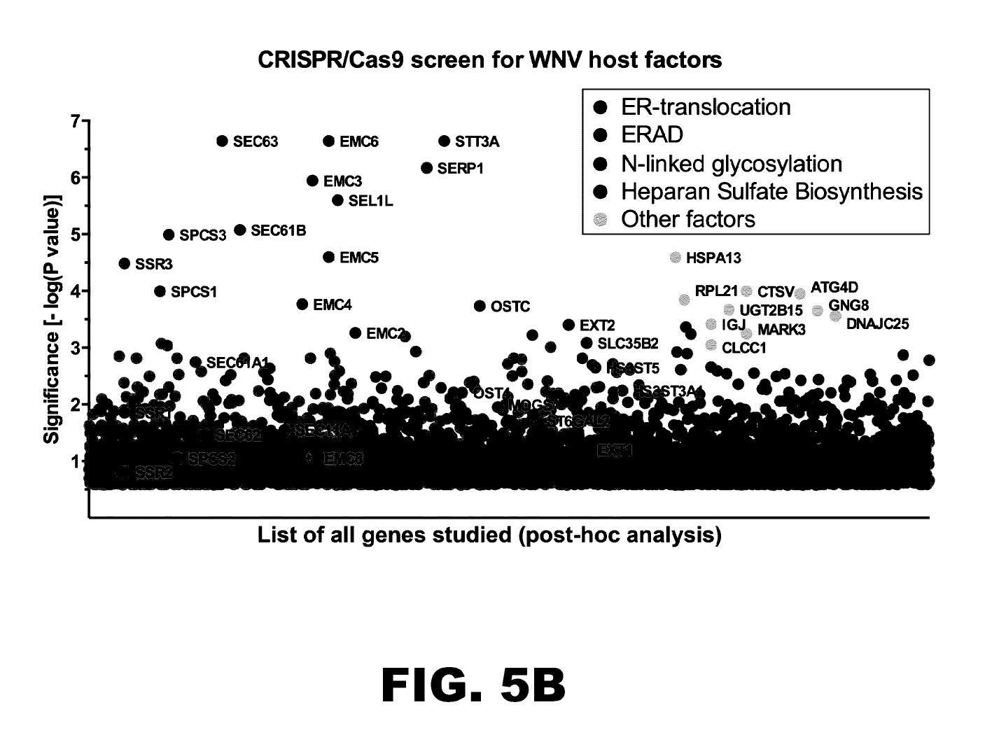

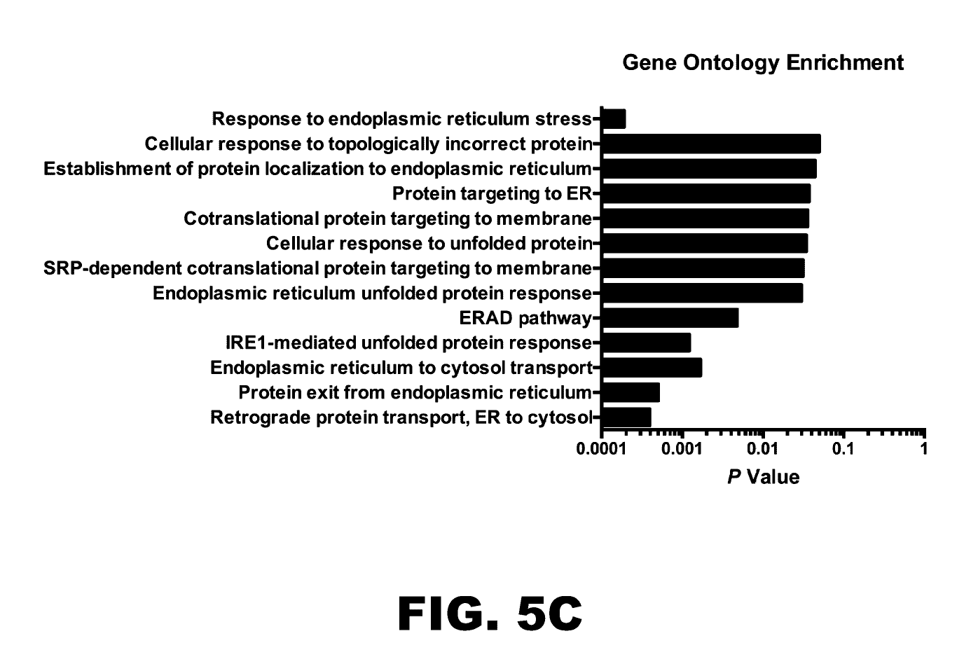

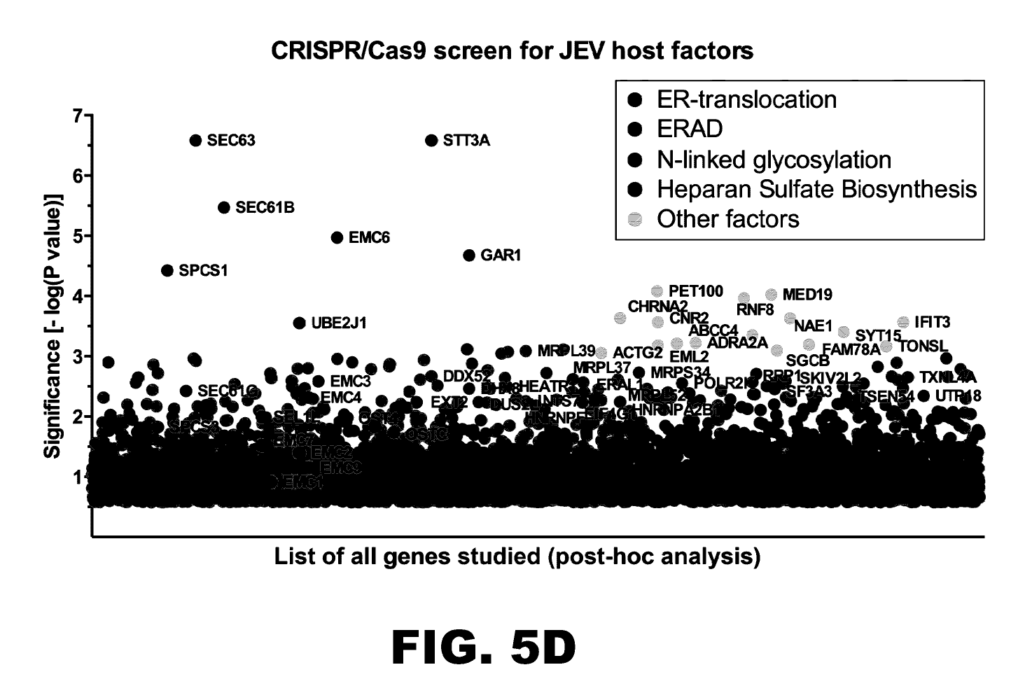

To identify genes required for infection and to overcome off-target effects associated with RNA silencing-based screens, we performed genome-wide CRISPR/Cas9 gene-editing screens in human 293T cells with WNV and JEV. The CRISPR/Cas9 system uses small guide RNAs (sgRNA) that facilitate sequence-dependent insertion or deletion of nucleotides, which enables functional knockout of both alleles in diploid mammalian cells^{5,8}. We designed an inhibition of cytopathic effect screen to identify genes that were required for WNV (strain New York 2000) or JEV (strain 14-14-2) infection in human 293T cells expressing the Cas9 RNA-guided DNA endonuclease (FIG. 5A). We transduced 293T-Cas9 cells with a commercial library of 122,411 sgRNA targeting 19,050 genes; sgRNA were packaged into lentiviruses, pooled to create a master library, and transduced at a low multiplicity of infection to limit the number of sgRNAs in each cell. Lentivirus transduced cells were then either infected with WNV or JEV or left untreated, followed by culture for 14 days. In the absence of library lentivirus transduction, no cells in virus-infected cultures survived. Colonies of lentivirus-transduced 293T-Cas9 cells surviving WNV or JEV infection were expanded and pooled separately, and sgRNA were amplified by PCR, subjected to next-generation sequencing, and compared to the library generated from the uninfected cells cultured in parallel. For additional validation and comparison, we performed the screens with two technical replicates or on separate days.

Based on analysis of the uninfected cell library, the sgRNA coverage was ˜93% of human genes. In cells surviving WNV infection, on average, we obtained ˜100 sgRNA reads that showed ˜10- or greater fold enrichment (Table 1) in the surviving cell population. Prioritization of gene ‘hits’ was based on sequencing data showing multiple different sgRNA per gene, the number of sequencing reads per gene, the enrichment of a given sgRNA compared to the uninfected cell library, and the reproducibility across the technical and biological repeats. Based on these criteria, 45 genes (Table 2) were selected as candidates for validation. Gene ontology enrichment analysis suggested that the majority of these were involved in endoplasmic reticulum (ER)-associated functions including carbohydrate modification (OST4, OSTC, STT3A, and SERP1), translocation (SEC63, SEC61B, SSR1, SSR3, SPSC1, and SPSC3), and protein degradation (ERAD: SEL1L, EMC3, and EMC6) (FIG. 5B, FIG. 5C). Genes also were identified in the heparan sulfate biosynthesis pathway (EXT2, SLC36B2, HS3ST5, HST3ST3A1), which was not unanticipated given the role of heparans in enhancing cellular attachment of flaviviruses^{7,8}. JEV infection of lentivirus-transduced 293T-Cas9 cells also identified several of the same ER-associated gene ‘hits’ (e.g., STT3A, SEC63, SEC61B, EMC3, and EMC6) (FIG. 5D), suggesting that conserved host pathways are required by multiple flavivirus family members for optimal infectivity.

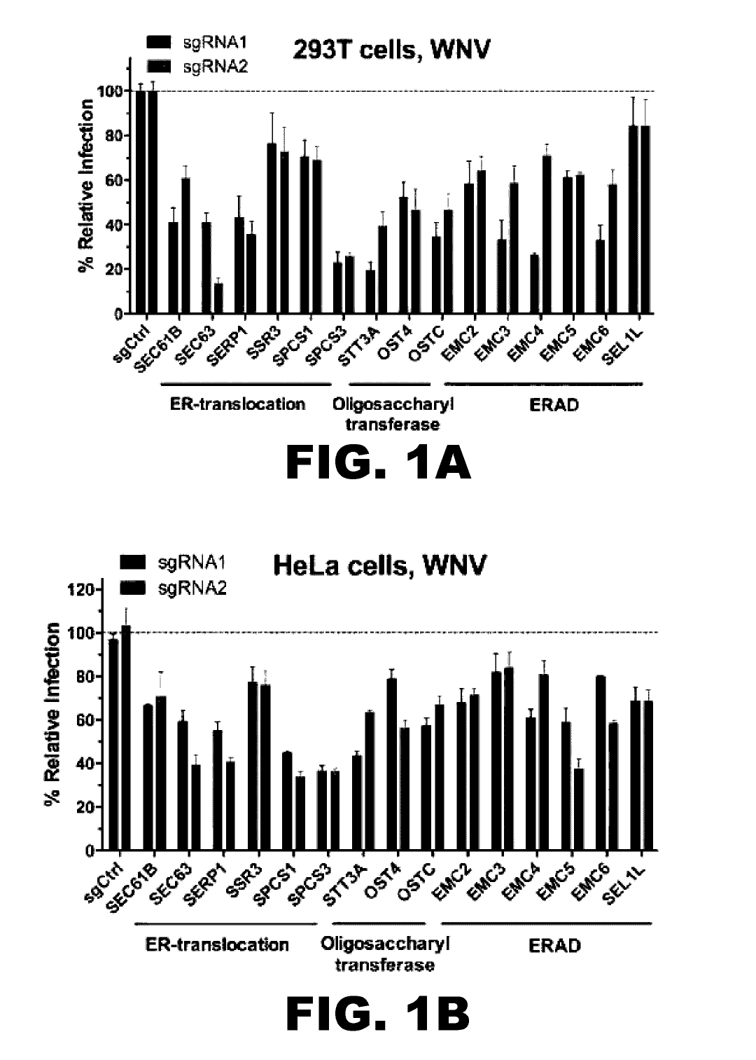

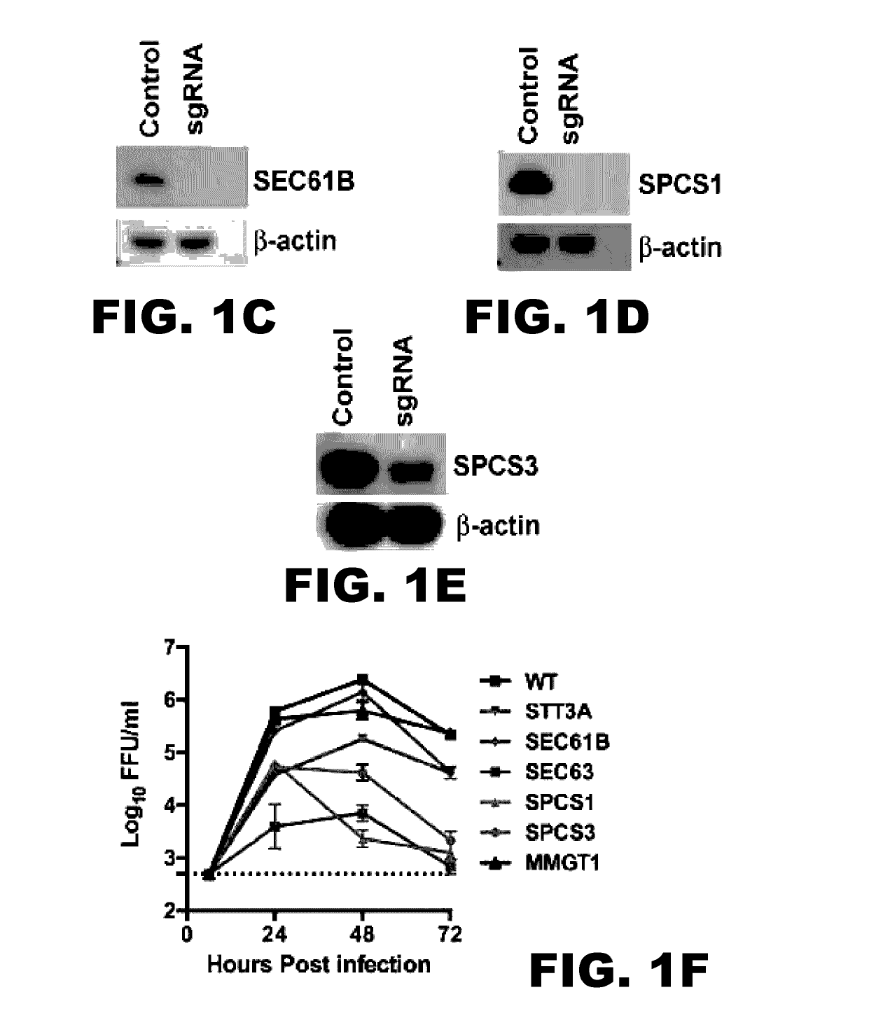

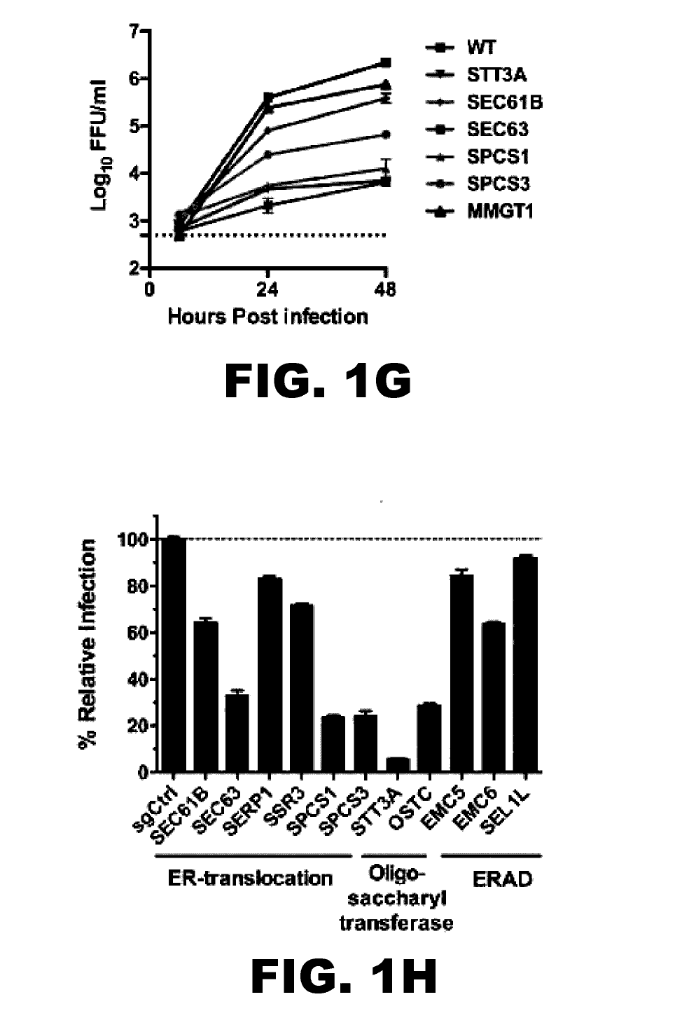

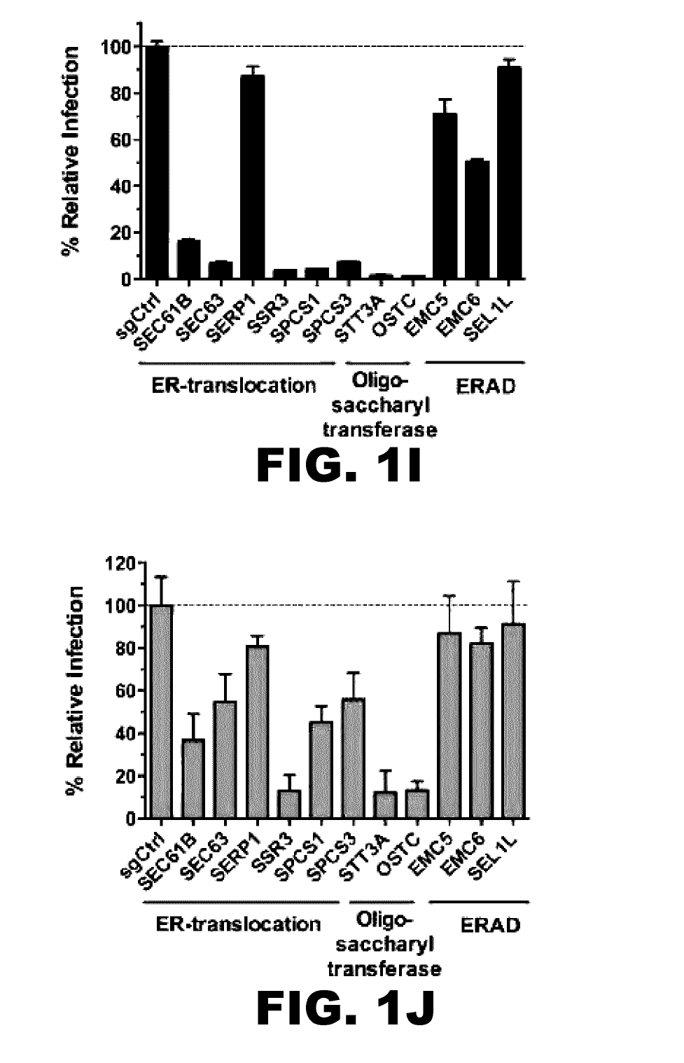

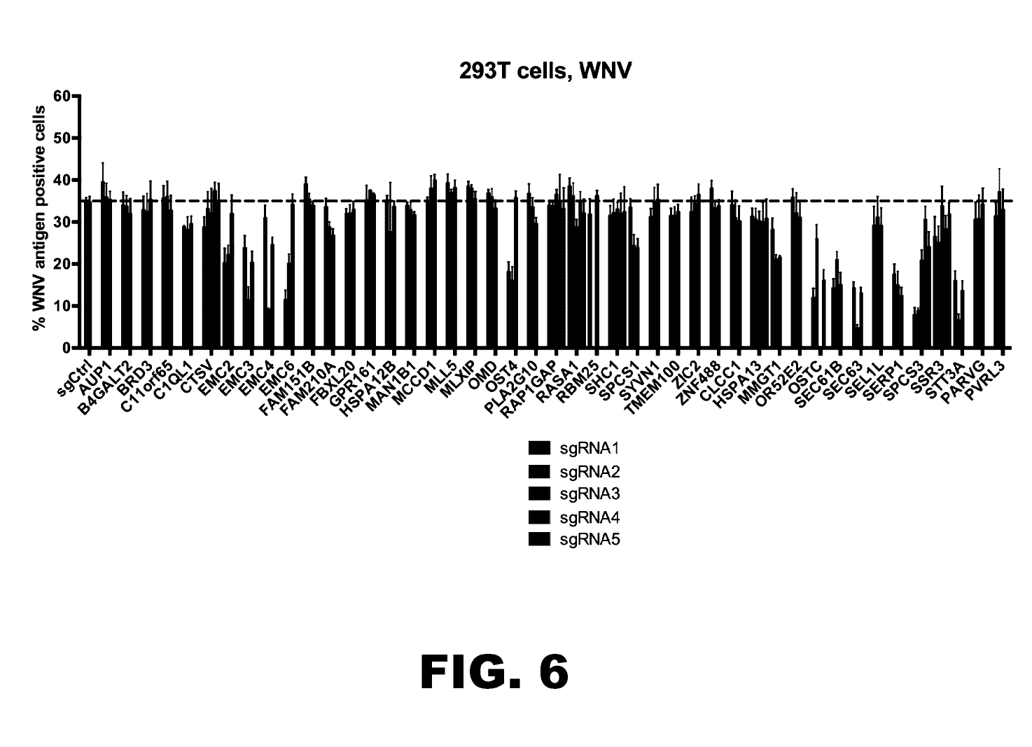

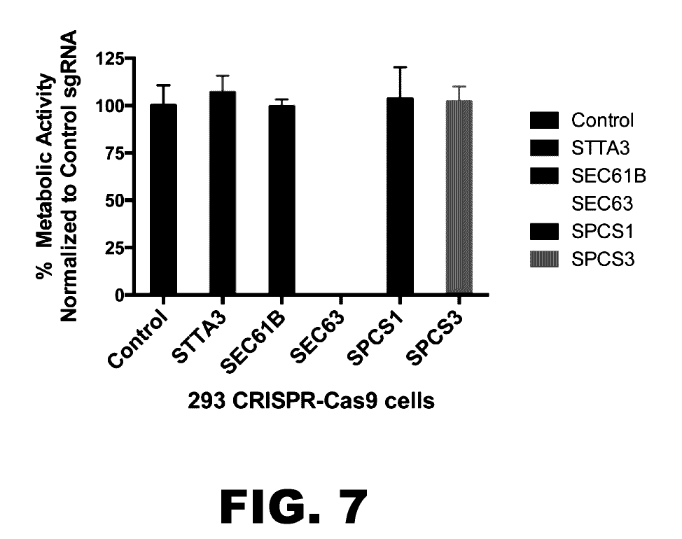

To validate the top 45 genes that emerged from computational analysis, 293T cells were transduced with a vector expressing Cas9, puromycin, and one of five different sgRNAs for each gene (Table 3). Four days after drug selection, bulk cells were infected with WNV at an MOI of 5, and 12 hours later infectivity was assessed by flow cytometry by staining for intracellular E protein expression. Notably, 12 genes (EMC3, EMC4, EMC6, SEL1L, SEC61B, SEC63, STT3A, OSTC, SERP1, SSR3, SPCS1, and SPCS2) were validated by this assay, with reduced infection observed in 293T cells expressing at least 2 different sgRNA against the same gene (FIG. 1A, FIG. 6). Importantly, sgRNA expression did not decrease WNV infection because of cellular toxicity, as cell viability was equivalent in the presence or absence of sgRNA at baseline (data not shown) or 24 h after infection (FIG. 7). Additional validation in a second cell line, human HeLa cells, with the two sgRNAs used to validate studies in 293T cells showed that editing of many of the 12 ‘hits’ also reduced WNV infection (FIG. 1B). We determined the knockout efficiency of our sgRNA in bulk-transduced cells by Western blotting for the selected genes (SEC61B, SPCS1, SPCS3) that we could obtain a validated antibody. These results confirmed that cells transduced with specific sgRNAs led to substantive decreases in protein expression (FIG. 1C, FIG. 1D, FIG. 1E).

We extended our studies to a multi-step growth assay in bulk gene-edited 293T cells with a subset of our validated genes; we selected STT3A, SEC63, SPSC1, or SPCS3 for further analysis because of their phenotypes in both 293T and HeLa cells after infection with WNV. Gene editing of STT3A, SEC63, SPSC1, or SPCS3 resulted in a 50 to 1,000-fold reduction in WNV yield at different time points after infection (FIG. 1F). Since the magnitude of the phenotype was large in this multi-step growth kinetic assay, these genes may have important roles in viral replication or cell-to-cell spread, which would be less apparent in single cycle infections, which were used in the primary screen.

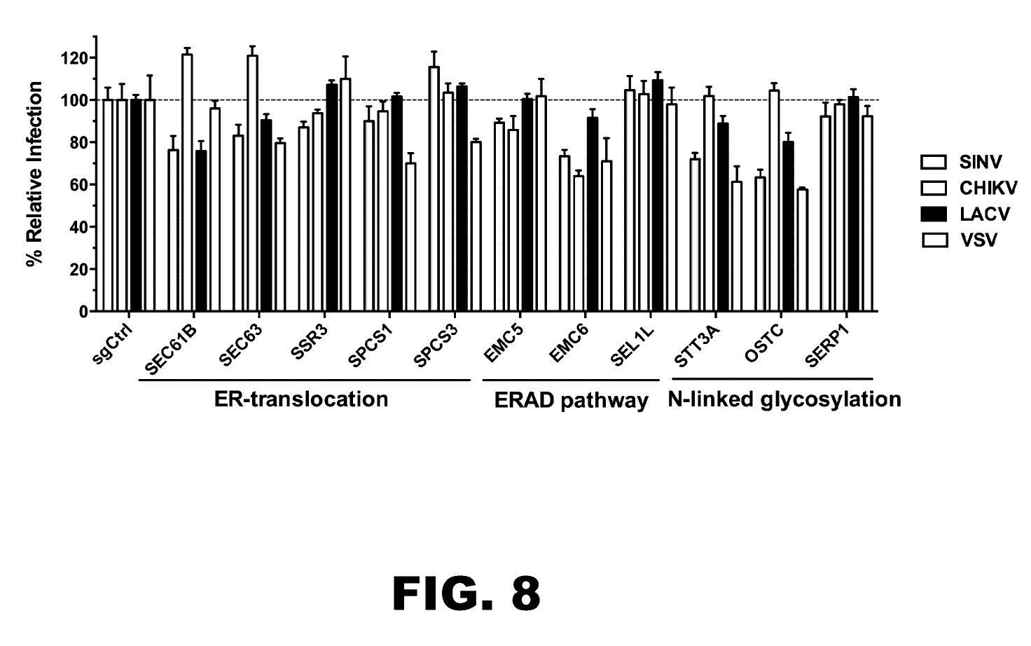

We next tested the role of the genes validated from the WNV screen against other globally relevant flaviviruses, including JEV, DENV serotype 2 (DENV-2), or YFV (FIG. 1H, FIG. 1I, FIG. 1J). Expression of 7 of the validated sgRNA also reduced infection of closely (JEV, ˜85% amino acid identity) and distantly (DENV and YFV, ˜45% amino acid identity) related flaviviruses. Of note, the magnitude of reduction of infection using the flow cytometric assay was greatest for DENV-2. Similar to the results with WNV, editing of STT3A, SEC63, SPSC1, or SPCS3 resulted in markedly (up to 1,000-fold) reduced JEV yield (FIG. 1G). An important role for these ER-associated genes was relatively specific to flaviviruses, as we observed less or no impact of gene editing on infection by unrelated positive or negative sense RNA viruses including alphaviruses, bunyaviruses, and rhabdoviruses (FIG. 8). One exception was genes modifying carbohydrate processing (STT3A and OSTC), which when edited, showed reduced infection of Sindbis and vesicular stomatitis viruses.

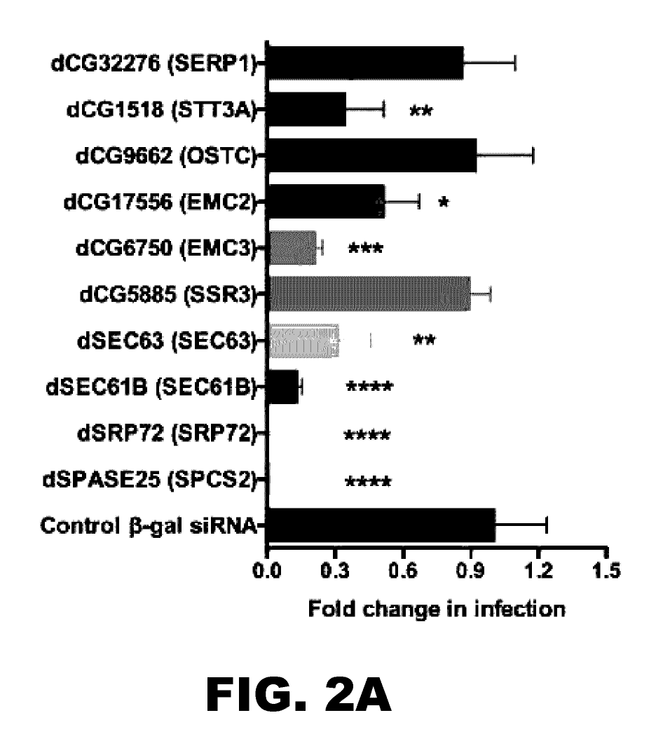

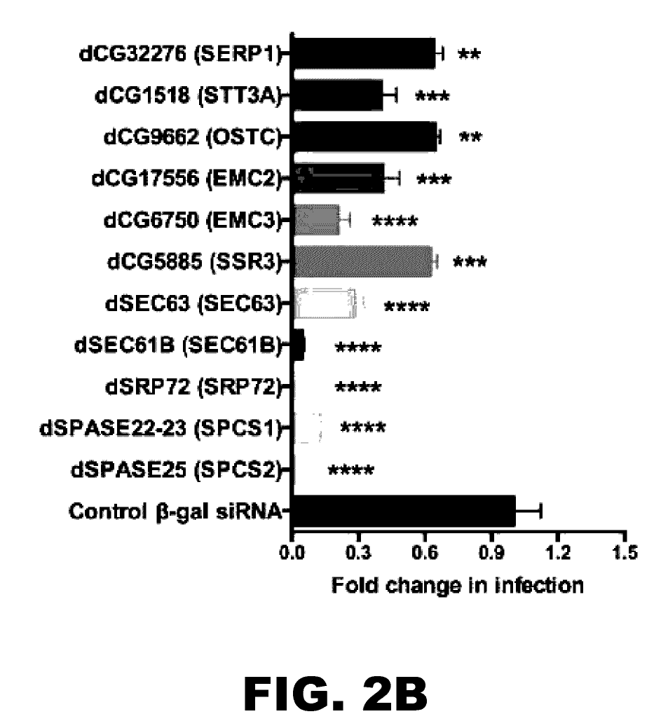

Given that many flaviviruses are arthropod-transmitted, we evaluated the roles of the gene orthologs in Drosophila insect cells using WNV and DENV-2. We tested 11 genes and found that silencing of Drosophila orthologs in the same ER-associated pathways of carbohydrate modification (dCG1518 [STT3A]), translocation and processing (dSEC63, dSEC61b, dSPCS2, dSRP72, dCG5885 [SSR3]), and protein degradation (ERAD: dCG17556 [EMC2] and dCG6750 [EMC3]) resulted in an loss of infection by WNV and DENV-2 (FIG. 2A, FIG. 2B); similar to the results seen in mammalian cells, the magnitude of the effects were larger for DENV compared to WNV. Importantly, silencing of these genes in insect cells did not affect viability (FIG. 2C). An analogous reduction in WNV infection was observed in AAG2 Aedes aegypti mosquito cells after gene silencing of SEC63, SRP72, and SPSC2 (FIG. 2D). Altogether, several of the validated genes that were required for efficient flavivirus infectivity in human cells had analogous impact on infection in insect cells.

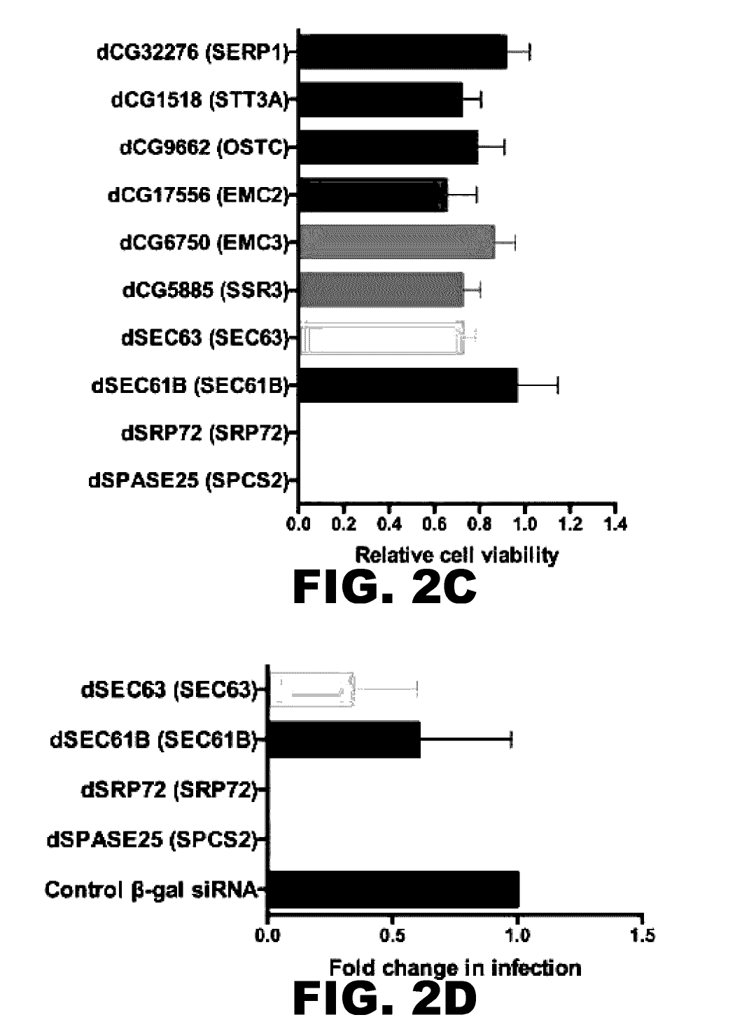

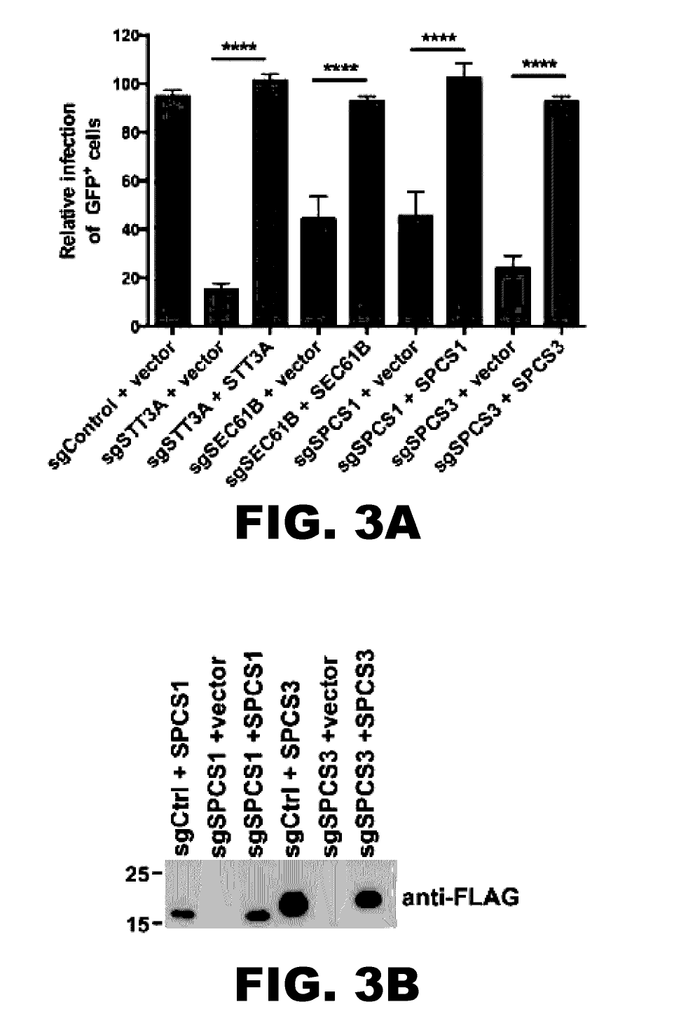

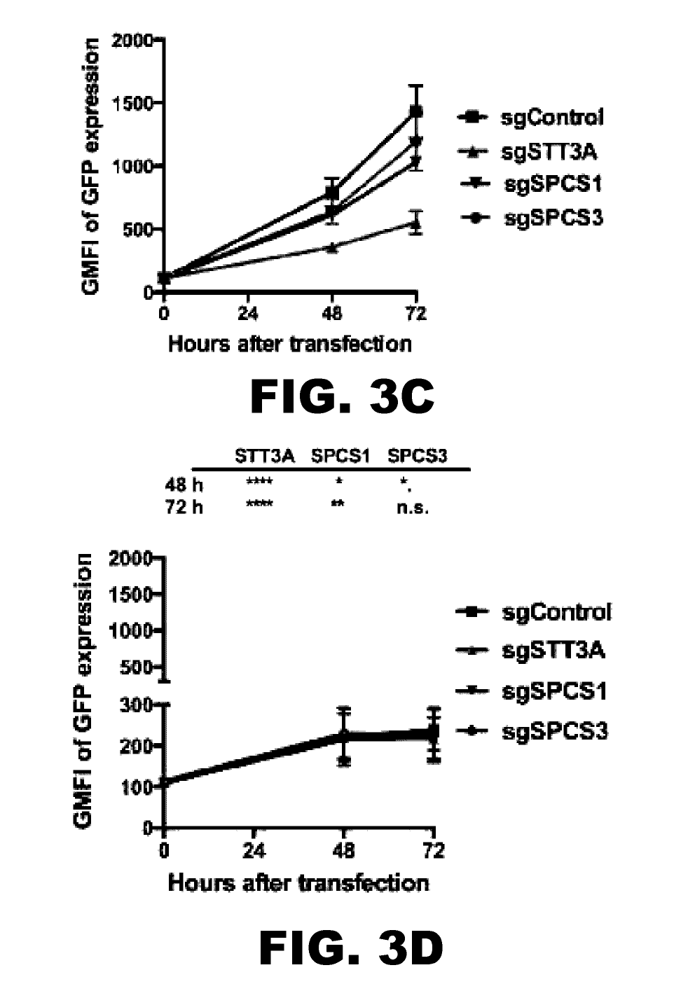

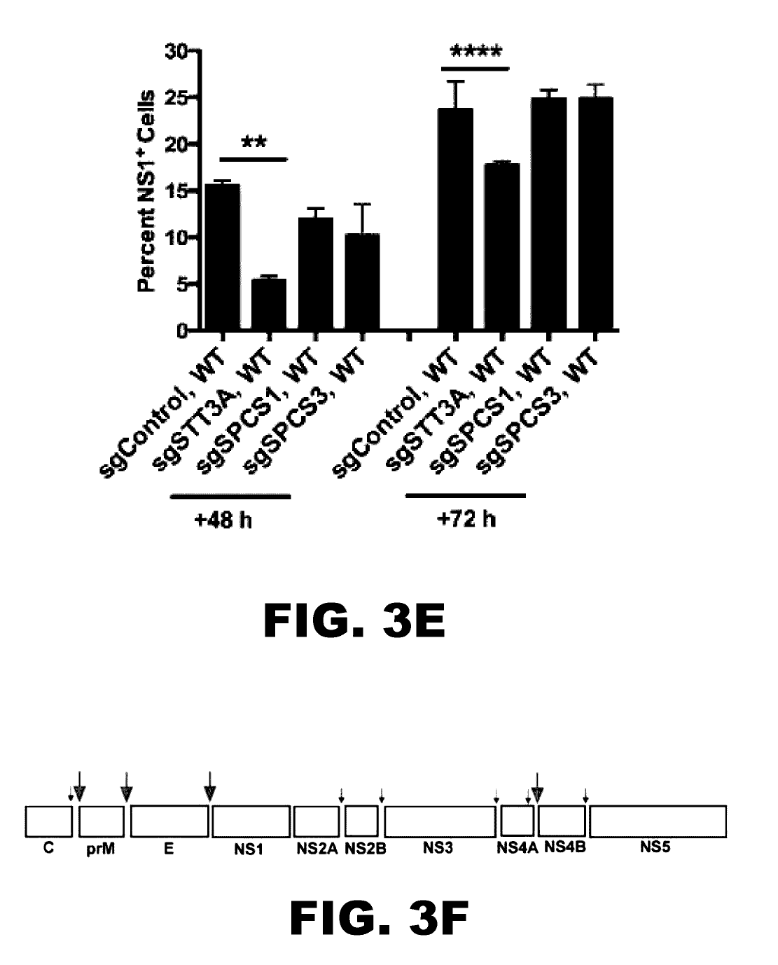

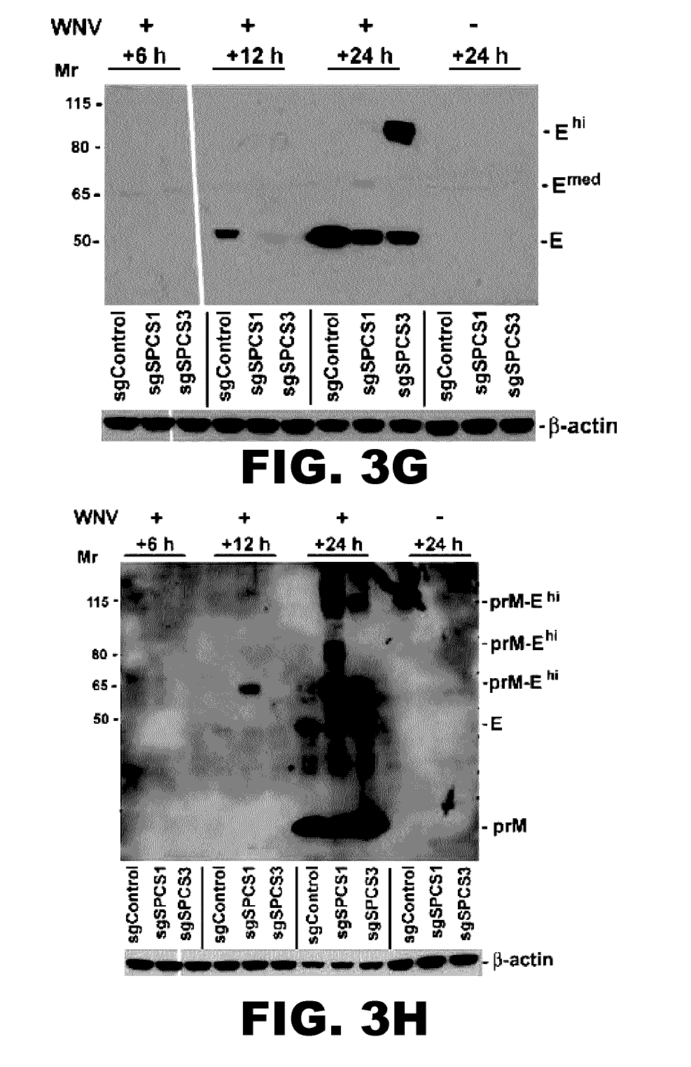

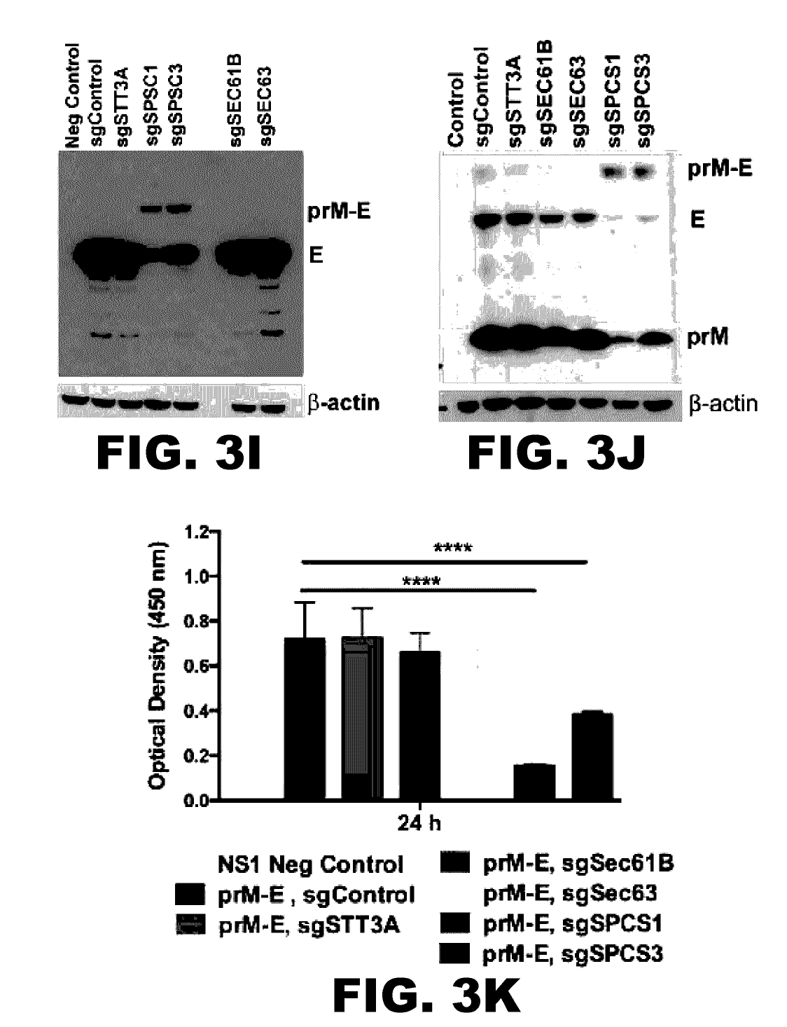

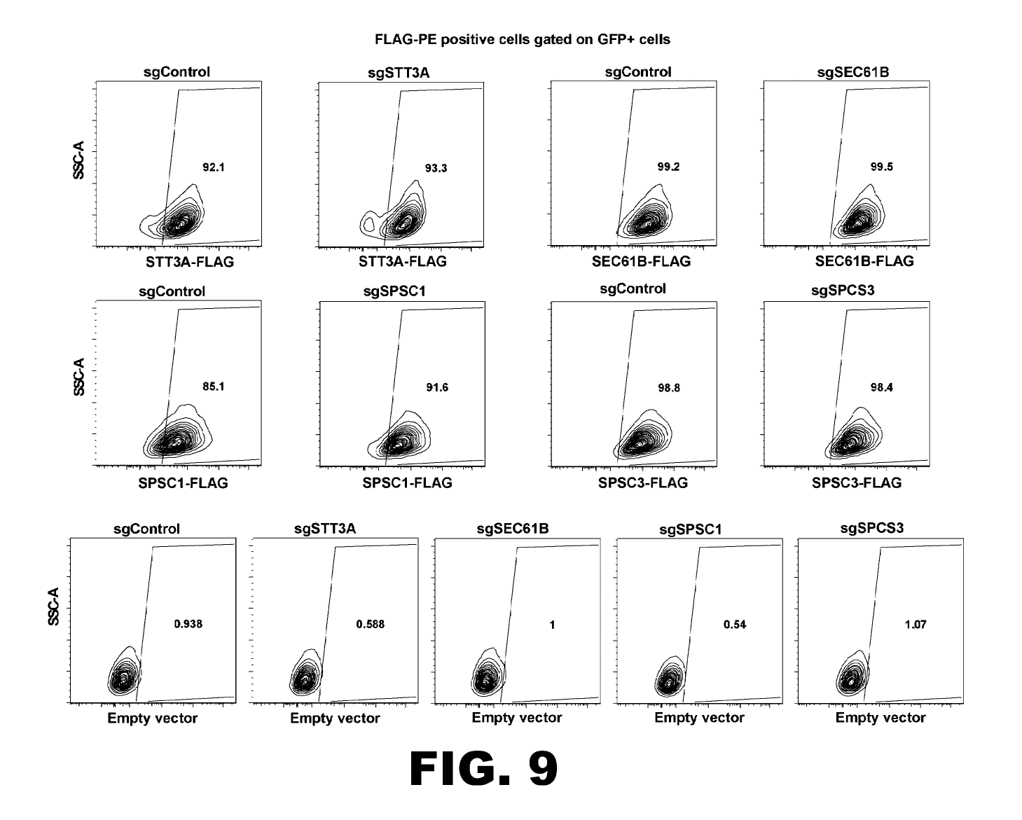

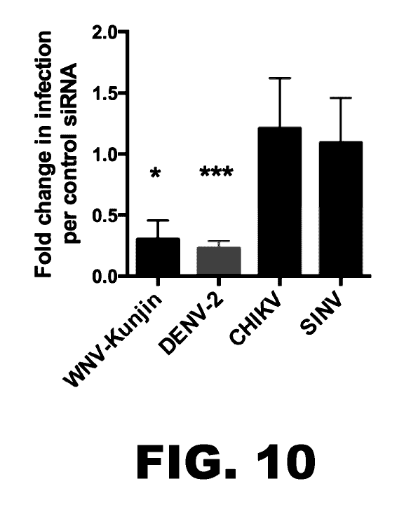

Although the observation of reduced infection of flaviviruses with multiple sgRNAs lessened the possibility of off-target gene editing effects, we validated our findings using trans-complementation with four ER-associated genes that regulate ER translocation (SEC61B, SPCS1, and SPCS3) or carbohydrate modification (STT3A) (FIG. 3A). Transfection of gene edited CRISPR cells with tagged versions of the wild-type (WT) alleles, which was confirmed by Western blotting (FIG. 3B, FIG. 9), resulted in enhanced WNV infection compared to control vector-transfected cells. Since we identified two of the three components of the Signal Peptide Processing Complex (SPSC1 and SPSC3) in the genome-wide CRISPR screen, we also tested whether SPSC2 was required for flavivirus infection using siRNAs. Indeed, depletion of SPSC2 in human U2OS cells led to reduced infection of WNV and DENV yet had no impact on alphavirus (CHIKV or SINV) infection (FIG. 10). However, we were unable to validate a SPCS2 gene-edited 293T cell despite attempts with multiple different sgRNA.

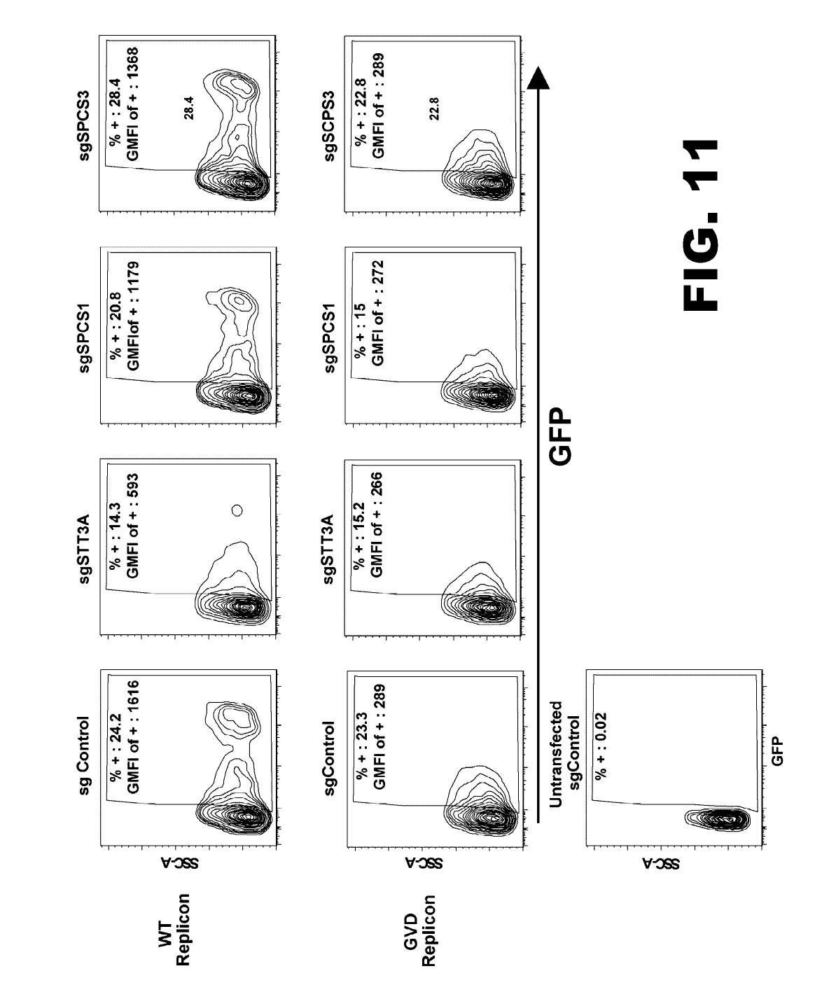

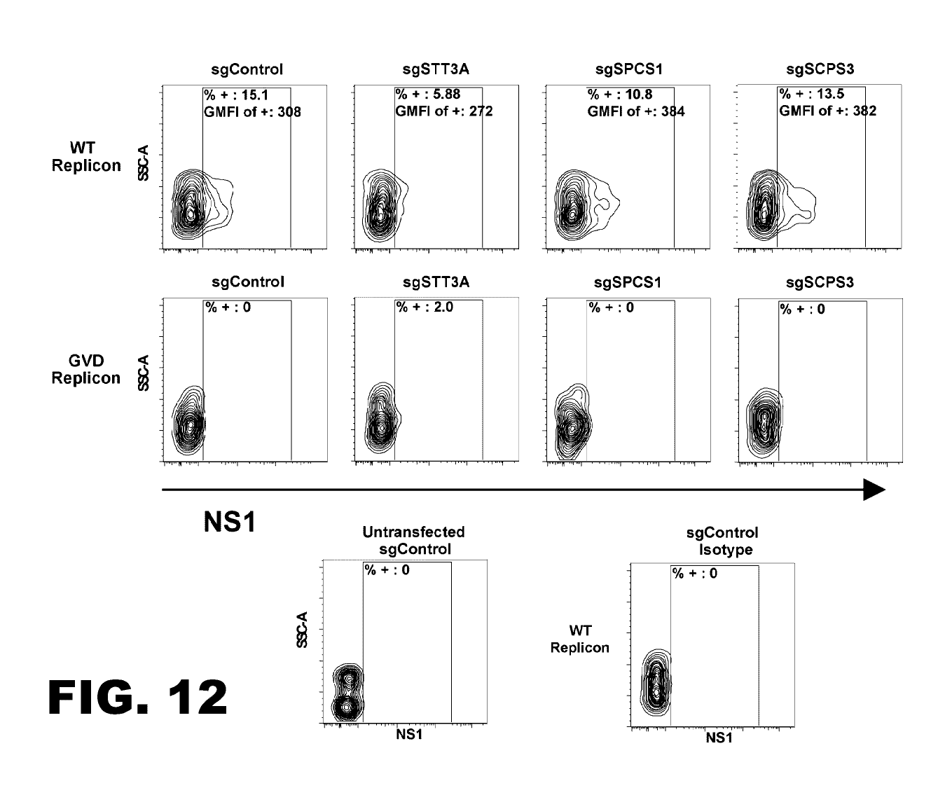

We next evaluated the stage in the viral lifecycle that was affected by loss of expression of several of the ER-associated genes that we validated. To determine whether genes were required for efficient translation and/or replication, we utilized WT and NS5 RNA-dependent RNA polymerase loss-of-function mutant (NS5 GDD→GVD⁹) WNV replicons (FIG. 3C, FIG. 3D). Transfection of cells with this cDNA-launched GFP-expressing replicon (GFP-NS1→NS5) results in the production of relatively low levels of viral RNA from an enhancer-less minimal CMV promoter that is independent of viral RNA replication, as measured by GFP expression (compare WT and GVD, FIG. 11). Viral RNA replication results in a significant increase in GFP-expression with time that depends on a functional RNA-dependent RNA polymerase, allowing a comparative measure of viral RNA replication. Accordingly, in control sgRNA cells, the mutant (NS5 GDD→GVD) replicon was translated but did not replicate and accordingly, the GFP signal remained dim over time (FIG. 3C, FIG. 3D), whereas the fluorescence intensity of GFP in control sgRNA cells transfected with the WT replicon increased over time. In STT3A gene-edited cells, GFP signal at 48 h and 72 h was diminished markedly compared to the control sgRNA cell, although no difference was seen with the non-replicating mutant GVD replicon. This suggests that STT3A is required for efficient replication but not translation. The results with the SPCS1 and SPCS3, however, were distinct. Despite the marked defect in viral yield by multi-step growth analysis (see FIG. 1F) in SPCS1 and SPCS3 gene-edited cells, near wild-type levels of GFP accumulation were observed after transfection of the WNV replicon. Analogous phenotypes were observed when we analyzed surface or intracellular levels of NS1 after transfection of WT replicons in the different gene-edited cells (FIG. 3E, FIG. 12).

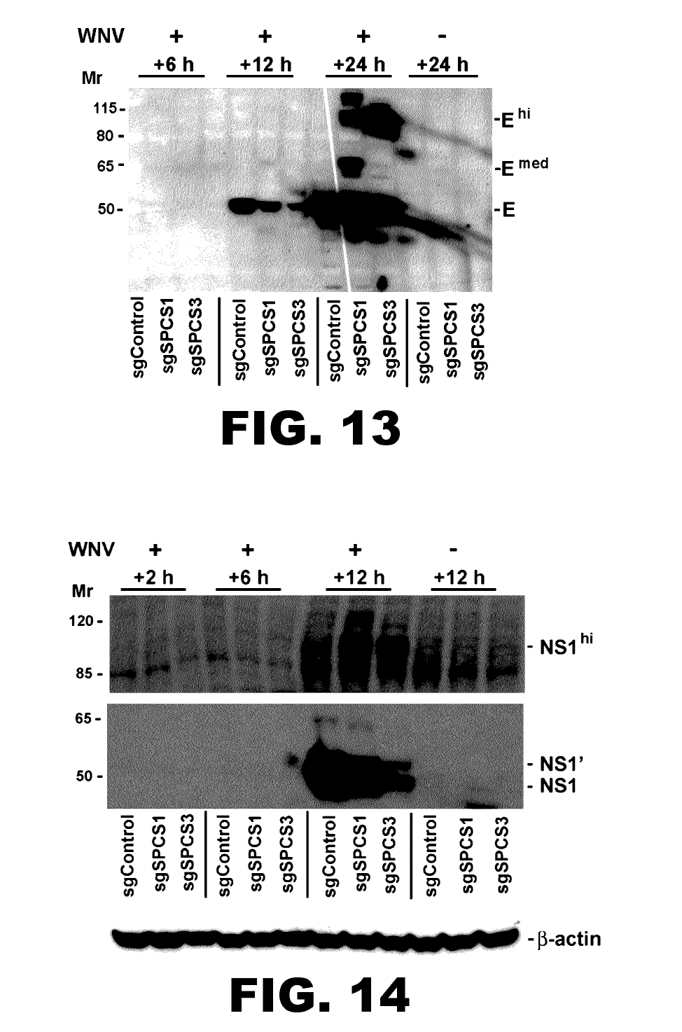

SPCS1 and SPCS3 have annotated functions as components of a signal peptidase complex^{10,11}, and SPCS1 reportedly is required for hepatitis C virus (HCV) assembly¹². Because a deficiency of SPCS1 and SPCS3 resulted in substantially reduced WNV and JEV yield while only modestly impacting replication of the WNV replicon, we speculated that the SPCS complex was a key host signalase required for efficient processing of the flavivirus polyprotein¹³. Flavivirus structural, NS1, and NS4B proteins require cleavage by unknown host signal peptidase(s), whereas the remaining non-structural proteins are cleaved in cis by the viral NS2B-NS3 protease (FIG. 3F and [symbol] ^{14,15}). To assess the role of the SPCS complex in polyprotein processing, gene-edited 293T cells were infected with WNV at a high multiplicity of infection. Lysates were prepared at different time points and subjected to Western blotting to monitor expression of the viral proteins. Studies with an anti-E protein antibody (hE16¹⁶) (FIG. 3G) revealed that less E protein was present in SPCS1 and SPCS3 gene-edited cells than the control cells at 12 h after WNV infection. By 24 h after infection, higher molecular weight aberrant bands reacted with the anti-E protein antibody, and this was more apparent in an over-exposed blot (FIG. 13). Of note, the pattern of high molecular weight bands that reacted with anti-E antibody were overlapping but not identical in SPSC1 and SPSC3 gene-edited cells, suggesting that the absence of an individual subunit could impact the efficiency of cleavage of a given target site in the WNV genome. Higher molecular weight yet non-identical bands also were apparent in the SPCS1 and SPCS3 gene-edited cells after blotting with an antibody (CR4293¹⁷) that binds to a shared determinant on prM and E (FIG. 3H). Consistent with these findings, less NS1 accumulated in WNV-infected cells that were gene-edited for SPCS1 or SCPC3, and a high molecular weight (˜100 kDa) band was apparent after blotting under highly denaturing conditions (boiled in SDS plus 5% β-mercaptoethanol) (FIG. 14). Collectively, this data suggests that components of the SPCS complex are required for proper processing of the viral prM, E, and NS1 proteins.

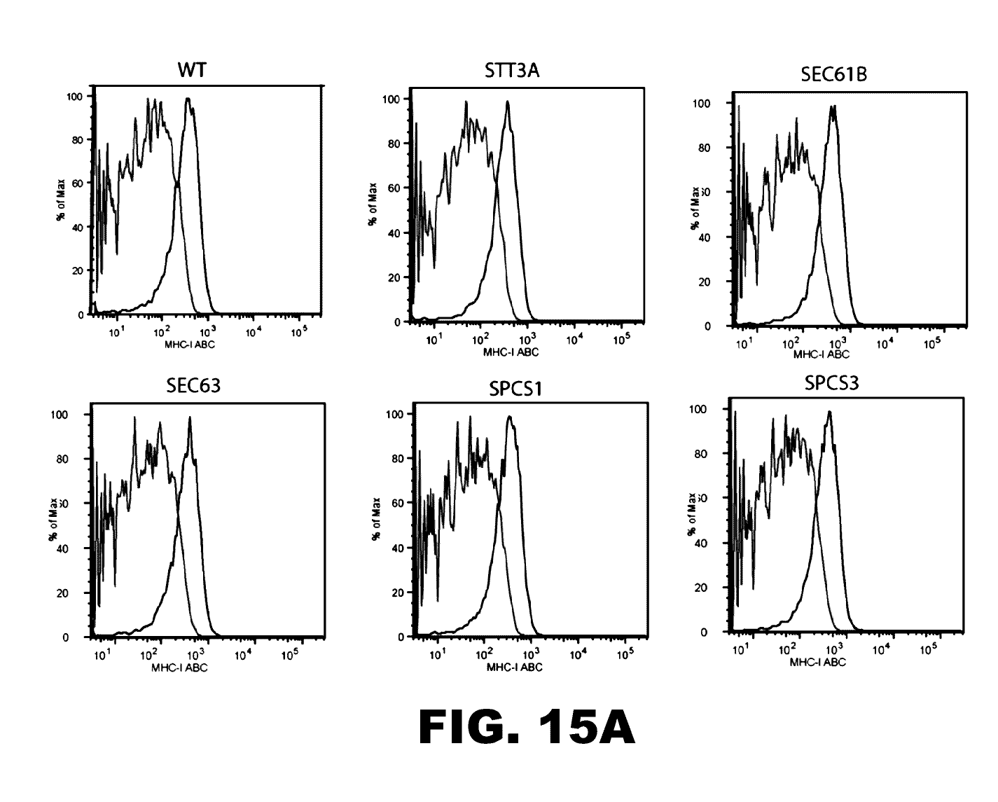

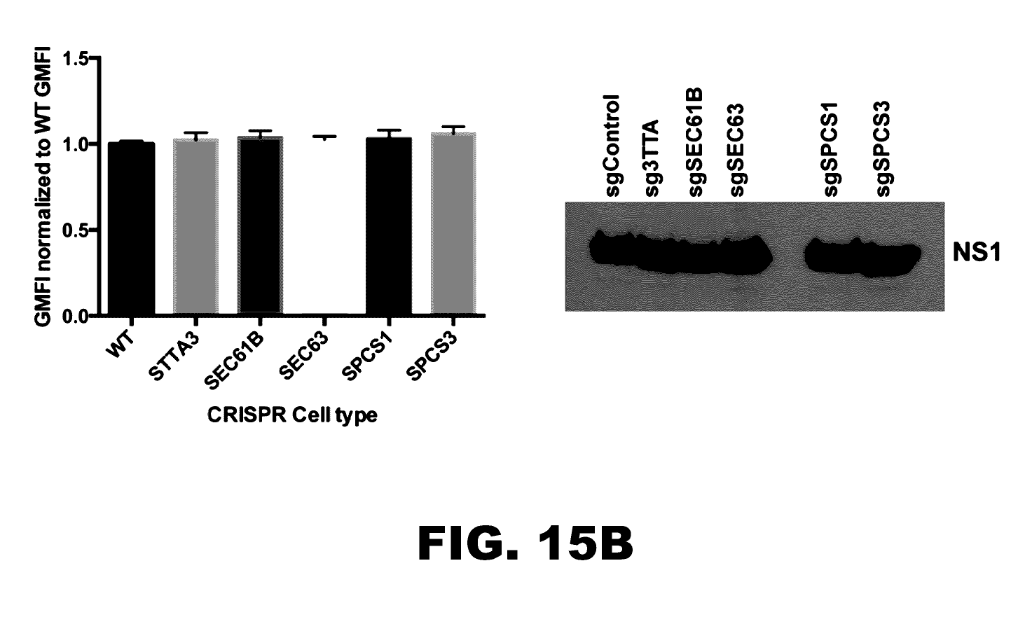

To isolate the effects of the SPCS complex on prM and E protein processing we used a plasmid encoding only the WNV prM-E structural genes, which upon translation and processing can produce secreted subviral particles (SVPs)¹⁸. We transfected this WNV prM-E plasmid into bulk gene-edited cell lines and assessed intracellular and extracellular production of prM and E proteins. Western blotting of cell lysates for E protein (˜55 kDa) showed both reduced levels and a higher molecular weight band (˜80 kDa) in cells deficient in SPCS1 or SPCS3 (FIG. 3I). These data suggest a defect in E processing from prM. This finding was corroborated by Western blotting with the CR4293 antibody, which showed reduced levels of prM and E and the emergence of the high molecular weight 80 kDa band in the SPCS1 and SPCS3 gene-edited cells (FIG. 3J). To confirm that the defect observed impacted particle assembly and release, we measured levels of secreted SVPs in the supernatant at 24 h after prM-E transfection. Substantive reductions were apparent in cells deficient in SPCS1 or SPCS3 (FIG. 3K). This effect of SPCS1 and SPCS3 on flavivirus protein processing was not global in nature, as we observed normal levels of endogenous HLA-A2 class I MHC antigen were present on the surface of these cells (FIG. 15A, FIG. 15B, left) and no impact on processing of a genetically engineered form of NS1 that depends on the endogenous CD33 leader sequence for processing (FIG. 15B, right).

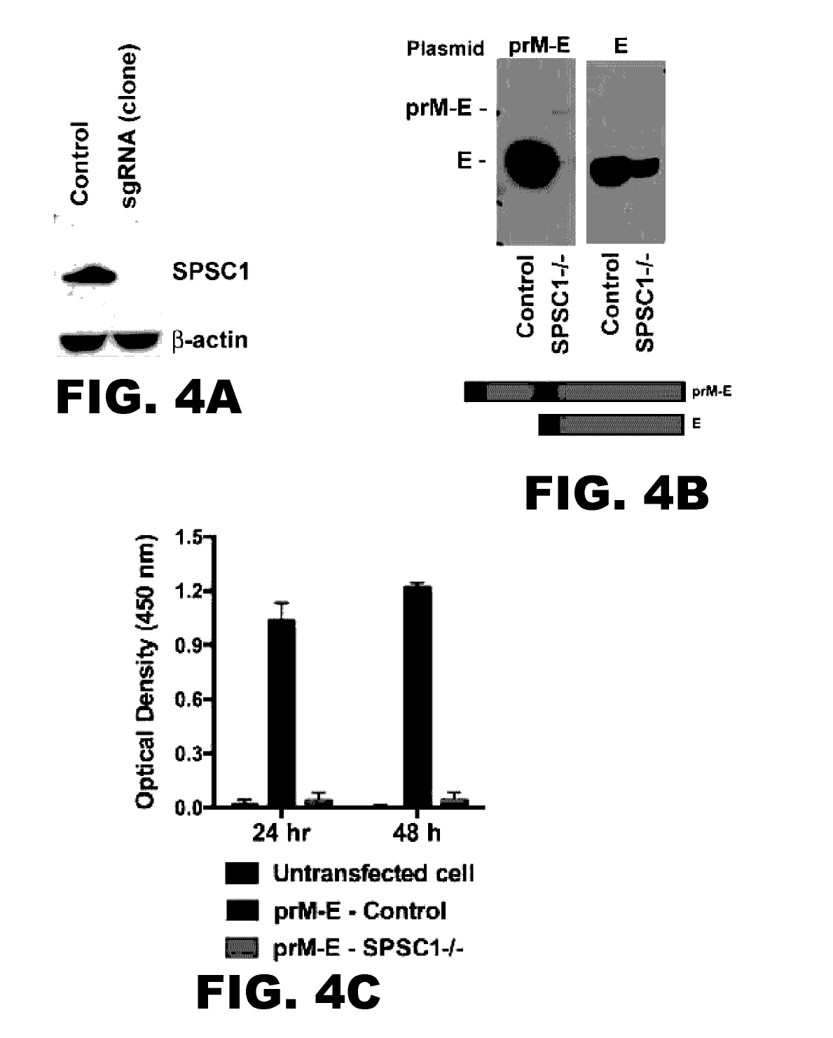

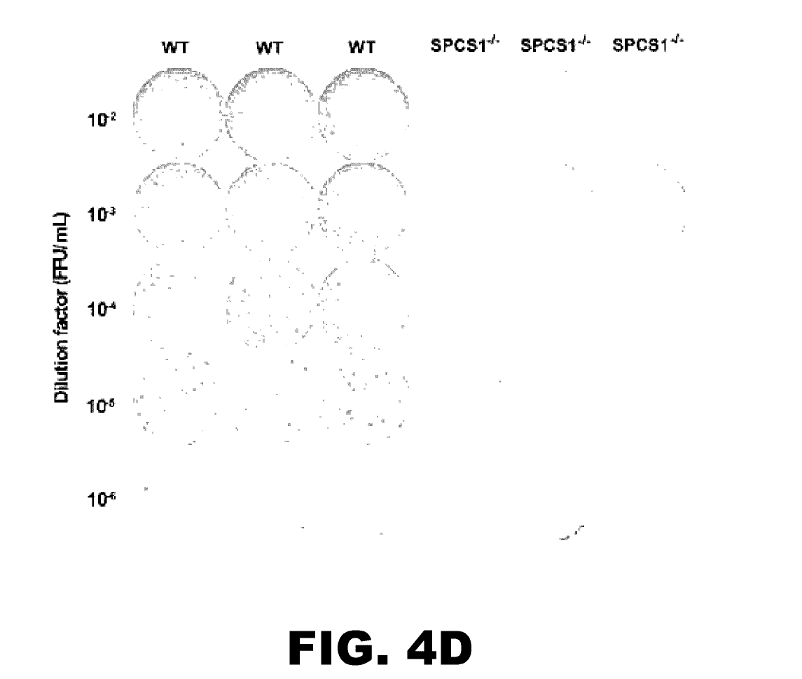

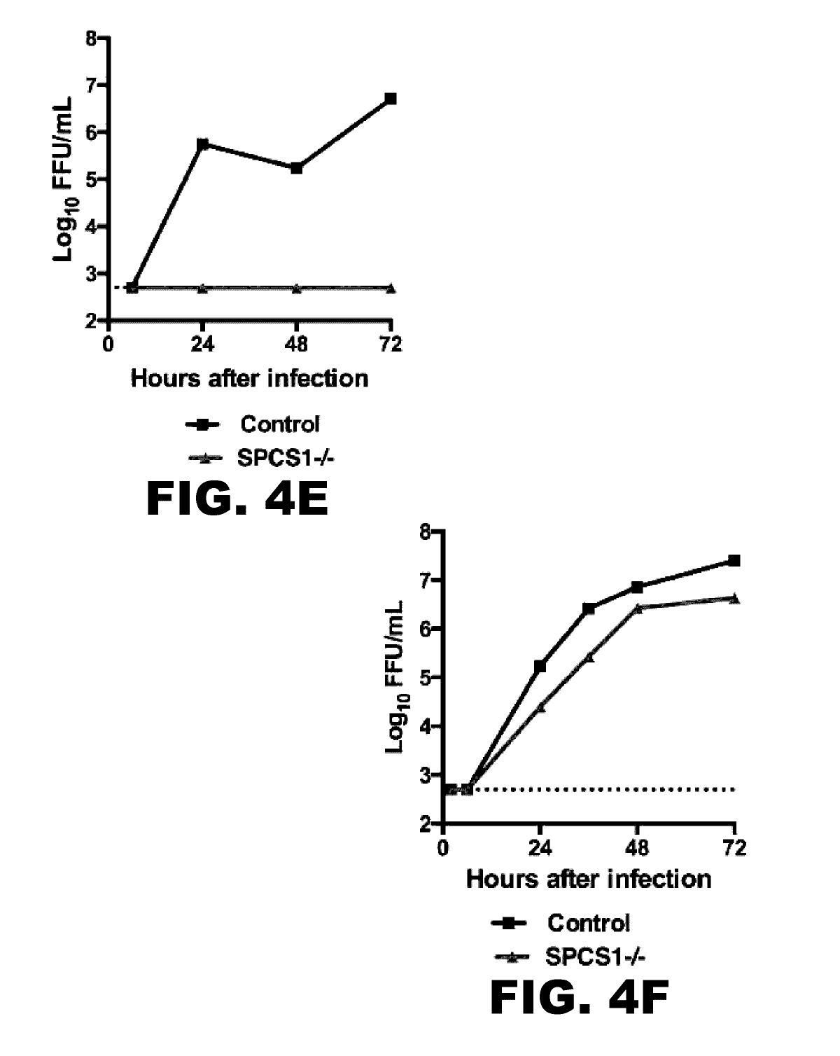

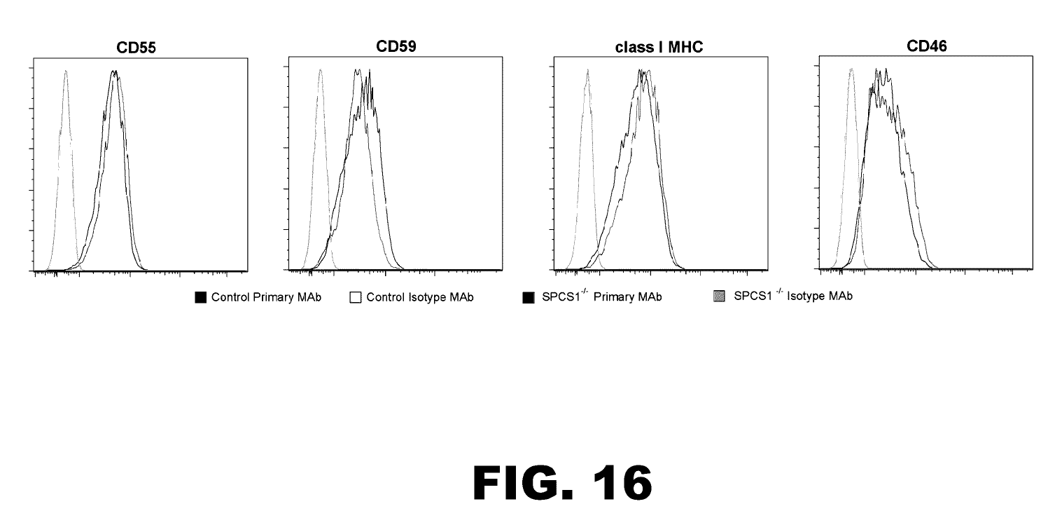

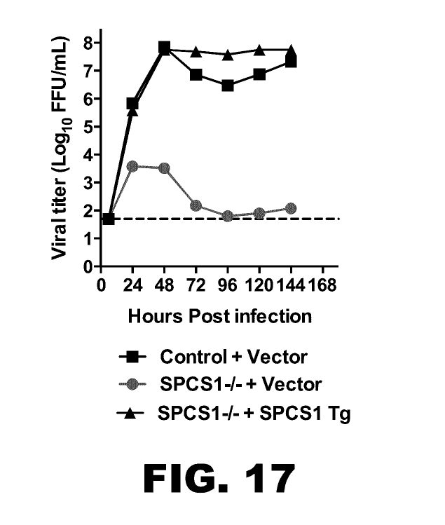

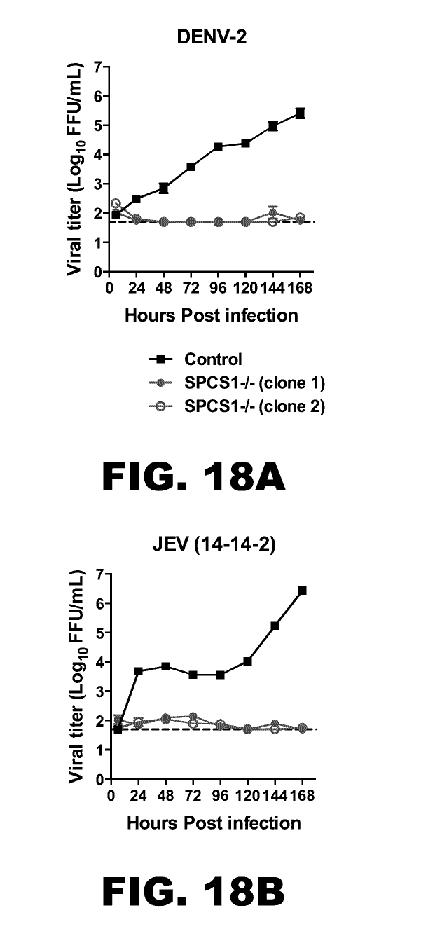

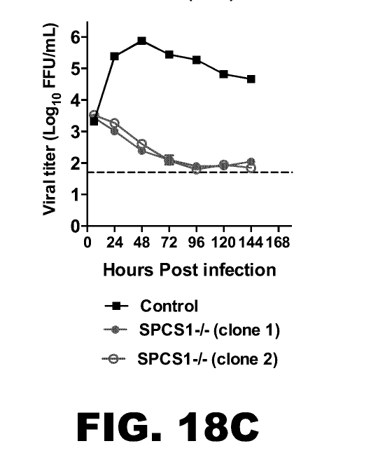

As bulk-selected cells might still retain one WT allele, we transduced sgRNA against SPCS1 or SPCS3 and selected clonal lines after limiting dilution cloning. SPCS3^{-/-} clones were not obtained despite several attempts, suggesting this gene may be essential. Several SPCS1^{-/-} clonal lines emerged and one was chosen for functional analysis after confirming both alleles contained non-sense mutations and/or deletions and the SPCS1 protein was absent (FIG. 4A). Transfection experiments with the single prM-E plasmid followed by Western blotting with anti-E MAb showed that loss of expression of SPSC1 was associated with almost complete loss of E protein expression, with a residual uncleaved prM-E band present (FIG. 4B, lanes 1-2). Consistent with this data, SVPs were not detected in the supernatant of SPCS1^{-/-} cells at 24 or 48 h after transfection, compared to high levels observed in control cells (FIG. 4C). Remarkably, infectious WNV and DENV failed to accumulate in the supernatant of SPCS1^{-/-} cells even 72 h after infection, with at least a 10,000-fold reduction in titer observed (FIG. 4D, FIG. 4E). The SPSC1^{-/-} cell line did not have general defects in processing of host proteins destined for the secretory pathway, as wild type levels of the complement regulator Membrane cofactor protein (MCP; CD46) were observed by Western blotting and cell surface expression of Decay accelerating factor (DAF; CD55), CD59, CD46, and class I MHC molecules anchored by glycophosphatidylinositol (GPI) or transmembrane anchors was unaffected (FIG. 16). Indeed, relatively smaller (5-fold) effects on yield in the supernatant of CHIKV, an unrelated alphavirus were observed in SPCS1^{-/-} cells (FIG. 4F).

In yeast, there exist parallel signal recognition targeting pathways, with specificity conferred by differences in the hydrophobic core of signal sequences^{19,20}. Given the specific reduction in processing and secretion of flavivirus structural proteins in SPCS1^{-/-} cells, we speculated that SPSC1 uniquely facilitated recognition of the hydrophobic character and/or internal leader sequence of flavivirus structural proteins. To test this hypothesis, we compared E protein expression when E was transfected as part of the prM-E plasmid or as a separate plasmid, with both E genes downstream of their native signal sequence (FIG. 4B, bottom). Whereas transfection of the prM-E in a single plasmid with an internal leader peptide did not result in efficient processing of the E protein, transfection of the E gene alone resulted in protein expression that was normal in size, albeit at slightly lower levels than observed in control cells (FIG. 4B, lanes 3-4).

Our screen preferentially identified genes with several ER-associated functions (carbohydrate modification, translocation, and ERAD) required for optimal flavivirus translation, polyprotein processing, and replication. Two of our top gene hits, the ER signal peptidase components SPCS1 and SPCS3 were required for flavivirus polyprotein processing as evidenced by a reduction in cleavage and accumulation of prM-E in the form of subviral particles; these latter experiments suggest that this complex is one of the previously unidentified host signalases required for viral polyprotein cleavage. Although the SPCS1 and SPCS3 were largely dispensable for flavivirus RNA replication, remarkably their loss did not impact surface expression or processing of host proteins including class I MHC molecules or complement regulatory factors, the processing of a recombinant flavivirus NS1 protein containing a heterologous human CD33 signal sequence, or alphavirus structural proteins or infectivity. This specificity suggests that the SPCS complex in mammalian and likely insect cells may represent one of several host signal peptidases that can promote cleavage of signal peptides for entry into the ER lumen, each with unique target site preference. Alternatively, SPCS1, SPSC2, and SPCS3 confer substrate specificity to a larger signal peptidase complex, and these proteins preferentially recognize flavivirus cleavage sites.

In separate interactome analysis, we observed that SPSC2 can bind to WNV NS2B (S. Cherry, unpublished results). Given that SPCS1 and SPCS3 were required for efficient cleavage of prM-E, and that flavivirus NS2B-3 has been reported to modulate the activity of the host signal peptidase cleavage at the C-prM junction²¹, flavivirus non-structural proteins (e.g., NS2B-3) might modulate the target specificity of the SPCS1/SPCS3 enzyme complex to facilitate additional cleavage events of the viral polyprotein.

A subset of our ER-related genes were identified in a prior RNAi screen with WNV in Drosophila cells⁴, and dSec61B was identified in an RNAi screen with DENV³. Virtually all of the human genes identified in our CRISPR screen involved in ER biology with insect orthologs also were required for optimal infection by two different flaviviruses, WNV and DENV, in insect cells. This suggests that flaviviruses utilize highly conserved host pathways in invertebrate and vertebrate cells to facilitate infection in multiple species. This is relevant because many flaviviruses (e.g., WNV) are highly promiscuous and can replicate in insects, birds, and many mammalian species. The ER is a particularly important site in the flavivirus lifecycle as its membranes support viral translation, polyprotein processing, replication, virion morphogenesis and carbohydrate modification of structural proteins. Thus, the identification of gene targets, especially those with enzymatic functions (e.g., signal peptidases) that are required for efficient flavivirus infection across phylogeny provide intriguing new candidates for pharmacological manipulation.

### References for Example 1

1 Suthar, M. S., Diamond, M. S. & Gale, M., Jr. West Nile virus infection and immunity. Nat Rev Microbiol 11, 115-128, doi:10.1038/nrmicro2950 nrmicro2950 [pii] (2013).
2 Krishnan, M. N. et al. RNA interference screen for human genes associated with West Nile virus infection. Nature 455, 242-245 (2008).
3 Sessions, O. M. et al. Discovery of insect and human dengue virus host factors. Nature 458, 1047-1050 (2009).
4 Yasunaga, A. et al. Genome-Wide RNAi Screen Identifies Broadly-Acting Host Factors That Inhibit Arbovirus Infection. PLoS Pathog 10, e1003914, doi:10.1371/journal.ppat.1003914 PPATHOGENS-D-13-01089 [pii] (2014).
5 Cong, L. et al. Multiplex genome engineering using CRISPR/Cas systems. Science 339, 819-823, doi:10.1126/science.1231143 science.1231143 [pii] (2013).
6 Jinek, M. et al. RNA-programmed genome editing in human cells. eLife 2, e00471, doi:10.7554/eLife.00471. 00471 [pii] (2013).
7 Chen, Y. et al. Dengue virus infectivity depends on envelope protein binding to target cell heparan sulfate. Nat Med 3, 866-871 (1997).
8 Lee, E., Hall, R. A. & Lobigs, M. Common E protein determinants for attenuation of glycosaminoglycan-binding variants of Japanese encephalitis and West Nile viruses. J Virol 78, 8271-8280 (2004).
9 Khromykh, A. A., Kenney, M. T. & Westaway, E. G. trans-Complementation of flavivirus RNA polymerase gene NS5 by using Kunjin virus replicon-expressing BHK cells. J Virol 72, 7270-7279 (1998).
10 Evans, E. A., Gilmore, R. & Blobel, G. Purification of microsomal signal peptidase as a complex. Proc Natl Acad Sci USA 83, 581-585 (1986).
11 Meyer, H. A. & Hartmann, E. The yeast SPC22/23 homolog Spc3p is essential for signal peptidase activity. J Biol Chem 272, 13159-13164 (1997).
12 Suzuki, R. et al. Signal peptidase complex subunit 1 participates in the assembly of hepatitis C virus through an interaction with E2 and NS2. PLoS Pathog 9, e1003589, doi:10.1371/journal.ppat.1003589 PPATHOGENS-D-13-00314 [pii] (2013).
13 Lindenbach, B. D., Murray, C. L., Thiel, H. J. & Rice, C. M. in Fields Virology Vol. 1 (eds D. M. Knipe & P. M. Howley) 712-746 (Lippincott Williams & Wilkins, 2013).
14 Chambers, T. J., Grakoui, A. & Rice, C. M. Processing of the yellow fever virus nonstructural polyprotein: a catalytically active NS3 proteinase domain and NS2B are required for cleavages at dibasic sites. J Virol 65, 6042-6050 (1991).
15 Falgout, B., Pethel, M., Zhang, Y. M. & Lai, C. J. Both nonstructural proteins NS2B and NS3 are required for the proteolytic processing of dengue virus nonstructural proteins. J Virol 65, 2467-2475 (1991).
16 Oliphant, T. et al. Development of a humanized monoclonal antibody with therapeutic potential against West Nile virus. Nature Medicine 11, 522-530 (2005).
17 Throsby, M. et al. Isolation and characterization of human monoclonal antibodies from individuals infected with West Nile Virus. J Virol 80, 6982-6992 (2006).
18 Schalich, J. et al. Recombinant subviral particles from tick-borne encephalitis virus are fusogenic and provide a model system for studying flavivirus envelope glycoprotein functions. J Virol 70, 4549-4557 (1996).
19 Hann, B. C. & Walter, P. The signal recognition particle in S. cerevisiae. Cell 67, 131-144, doi:0092-8674(91)90577-L [pii] (1991).
20 Ng, D. T., Brown, J. D. & Walter, P. Signal sequences specify the targeting route to the endoplasmic reticulum membrane. J Cell Biol 134, 269-278 (1996).
21 Stocks, C. E. & Lobigs, M. Signal peptidase cleavage at the flavivirus C-prM junction: dependence on the viral NS2B-3 protease for efficient processing requires determinants in C, the signal peptide, and prM. J Virol 72, 2141-2149 (1998).
22 Ma, H. et al. A CRISPR-Based Screen Identifies Genes Essential for West-Nile-Virus-Induced Cell Death. Cell Rep, doi:S2211-1247(15)00675-0 [pii]. 10.1016/j.celrep.2015.06.049 (2015).

### Methods for Example 1

### Cells and Viruses

Vero, BHK21, HeLa, U205, and 293T cells were cultured at 37° C. in Dulbecco's Modified Eagle Medium supplemented with 10% fetal bovine serum (FBS). C6/36 Aedes albopictus cells were cultured at 28° C. in L15 supplemented with 10% FBS and 25 mM HEPES pH 7.3. Drosophila DL1 cells were cultured at 28° C. in Schneiders' medium supplemented with 10% FBS as described¹. The following viruses were used in screening and validation studies: WNV (New York 2000), WNV (Kunjin), JEV (14-14-2), DENV-2 (16681 and New Guinea C strains), YFV (17D), LACV (original strain), VSV (Indiana), and SINV (Toto). All viruses were propagated in Vero or C6/36 cells and titrated by standard plaque or focus-forming assays².

sgRNA Library and Screen.

A pooled library encompassing 122,411 different sgRNA against 19,050 human genes was derived by the Zheng laboratory³ and obtained from a commercial source (Addgene). The library was packaged using a lentivirus expression system. 293T cells were transfected using Fugene®HD (Promega). Forty-eight hours after transfection, supernatants were harvested, clarified by centrifugation (300 g×5 min), filtered, and aliquotted for storage at −80° C.

For the screen, we generated 293T-Cas9 cells by transfecting the lentiCas9-Blast plasmid (Addgene #52962) using Fugene®HD transfection reagent and blasticidin selection. These 293T-Cas9 cells (5×10⁷) were infected with lentiviruses encoding individual sgRNA at a multiplicity of infection (MOI) of 0.1. Two days later, after extensive washing, transduced cells were infected with WNV or JEV at an MOI of 1 and then incubated for 14 days. In parallel, untransduced 293T-Cas9 cells were infected to ensure virus-induced infection and cell death. The experiments were performed parallel as either duplicate or triplicate technical replicates, and for WNV the screen was repeated in an independent biological experiment.

Genomic DNA was extracted from the cells that survived WNV or JEV infection, and sgRNA sequences were amplified. The amplified product was subjected to next generation sequencing using an Illumina Hi-Seq 2500 platform, and the sgRNA sequences against specific genes were recovered after removal of the tag sequences.

Gene Validation.

Bioinformatic analysis was used to determine the sgRNA sequences that were enriched in the cells that survived WNV or JEV infection. This was achieved using a program, and accounted for the number of sequencing reads per gene, and the enrichment of a given sgRNA compared to the uninfected cell library, which was prepared in parallel. A further cut-off of candidate genes was made manually and reflected the reproducibility across the different technical and biological repeats. From this, we identified 45 top ‘hits’. These candidate genes were tested for validation by designing 4 to 5 independent sgRNA per gene as oligonucleotides and cloning them into the pLentiCRISPR v2 (Addgene plasmid 52961) per the manufacturer's instructions. A control sgRNA was designed. Plasmids were transfected into 293T or HeLa cells using Lipofectamine 2000 (Life Technologies) and puromycin was added one day later. Three days later, puromycin was removed, and cells were allowed to recover for three additional days prior to infection with different viruses.

For flow cytometric analyses, gene-edited 293T or HeLa cells were infected with WNV (MOI, 5), JEV (MOI, 50), DENV-2 (MOI, 3), YFV (MOI, 3), CHIKV, SINV, LACV, or VSV and analyzed 12 or 24 hours later depending on the individual virus. Cells were fixed with 1% paraformaldehyde (PFA, Electron Microscopy Sciences) diluted in PBS for 20 min at room temperature and permeabilized with Perm buffer (HBSS (Invitrogen), 10 mM HEPES, 0.1% (w/v) saponin (Sigma), and 0.025% NaN₃ (Sigma)) for 10 min at room temperature. Cells then were rinsed one additional time with Perm buffer. Cells (5×10⁴) were transferred to a U-bottom plate and incubated for 1 h at 4° C. with 1 mg/ml of the following virus-specific or isotype control mouse antibodies. After washing, cells were incubated with an Alexa Fluor 647-conjugated goat anti-mouse or anti-human IgG (Invitrogen) for 1 h at 4° C. Cells were fixed in 1% PFA in PBS, processed on a FACS Array (BD Biosciences) and analyzed using FlowJo software (Tree Star).

Validation also was performed by an infectious virus yield assay. Gene-edited 293T cells were infected with WNV or JEV (MOI, 0.01). Supernatants were harvested at specific times after infection and focus-forming assays were performed in 96-well plates as described previously⁴. Following infection, cell monolayers were overlaid with 100 ml per well of medium (1×DMEM, 4% FBS) containing 1% carboxymethylcellulose, and incubated for 16 to 18 hours at 37° C. with 5% CO₂. Cells were then fixed by adding 100 ml per well of 1% paraformaldehyde directly onto the overlay at room temperature for 40 minutes. Cells were washed twice with PBS, permeabilized (in 1×PBS, 0.1% saponin, and 0.1% BSA) for 20 minutes, and incubated with cross-reactive antibodies specific for WNV or JEV (mouse WNV E18⁵) E glycoprotein for 1 h at room temperature. After rinsing cells twice, cells were incubated with species-specific HRP-conjugated secondary antibodies (Sigma). After further washing, foci were developed by incubating in 50 ml/well of TrueBlue peroxidase substrate (KPL) for 10 min at room temperature, after which time cells were washed twice in water. Well images were captured using Immuno Capture software (Cell Technology Ltd.), and foci counted using BioSpot software (Cell Technology Ltd.).

Insect Cell Infections.

dsRNAs were generated as described⁶. To silence genes using RNAi, insect cells were passaged into serum-free media containing dsRNAs targeting the indicated genes. Cells were serum-starved for one hour, after which complete media was added and cells were incubated for 3 days. Cells were infected with WNV (Kunjin strain) at an MOI of 4 or DENV-2 (NGC strain) at an MOI of 1 for 30 h and then processed for microscopy with automated image analysis as described⁷.

siRNA Treatments in Human Cells.

Human U2OS cells were transfected with siRNAs against either control or SPCS2 for three days and infected with WNV (KUN) (MOI, 1) for 18 h, or SINV (MOI, 0.1) and CHIKV (MOI, 2) for 20 h, and processed for microscopy with automated image analysis as described⁷.

Gene Ontology Enrichment Analysis.

Enrichment analysis was performed on the 45 top candidates that were identified by CRISPR-Cas9 screening using Panther.

Replicon Transfection and Analysis.

The construction of WT and NS5 polymerase mutant (GDD→GVD) WNV replicons (lineage I, strain New York 1999) was based on a previously described cDNA launched molecular done system⁸. The backbone of this strategy, a plasmid containing a truncated WNV genome under the control of a CMV promoter (pWNV-backbone); was designed to be complemented via ligation of a structural gene DNA fragment; transfection of pWNV-backbone alone does not result in production of a self-replicating RNA molecule. Using overlap extension PCR and unique restriction endonuclease sites, pWNV-backbone was modified by the introduction of a fragment downstream of the CMV promoter encoding [5′UTR-cylization sequence of capsid-FMDV2a protease-signal sequence of E-NS1] to complement the [NS2→NS5-3′UTR] already present in the pWNV-backbone plasmid, generating the replicon plasmid pWNVI-rep. The reporter gene GFP then was cloned upstream of the FMDV2a protease sequence via a unique Mlul site to generate pWNVI-rep-GFP. The construction and organization of this WNVI replicon is analogous to a previously described lineage II WNV replicon (pWNVIIrep-GFP)⁹. Finally, QuikChange mutagenesis (Agilent Technologies) was used to delete the enhancer portion of the CMV immediate early enhancer/promoter, generating pWNVI-minCMV-rep-GFP, and to generate the GDD→GVD NS5 polymerase variant. Although the CMV enhancer/promoter combination commonly found in cloning vectors results in robust and constitutive expression, inclusion of only the minimal CMV promoter (no enhancer) results in low level expression¹⁰. As such, direct transfection of pWNVI-minCMV-rep-GFP results in a low GFP signal, which reflects translation of the RNA generated by DNA-dependent RNA translation. RNA polymerase-dependent replication of the WT (but not GVD mutant) replicon results in higher production of GFP over time. The eGFP is bracketed by the FMDV2a autocleavage site, and does not rely on host or viral proteases for processing. WT and NS5 GVD variants of pWNVI-minCMV-rep-GFP (200 ng) were transfected into 10 controller gene-edited 293T cells (96 well plates) using Lipofectamine 2000. At various times after transfection, cells were harvested, cooled to 4° C., stained sequentially with a biotinylated anti-9NS1¹¹ (or biotin anti-chikungunya virus negative control MAb) and Alexa 647 conjugated streptavidin. In some samples, cells were fixed with 4% paraformaldehyde in PBS (10 min, room temperature) and permeabilized with 0.1% saponin (w/v). Cells were processed for two-color flow cytometry using a FACSScan (Becton Dickinson).

prM-E or NS1 Plasmid Transfection.

CRISPR-Cas9 293T cells were transfected with a pWN-AB plasmid expressing prM and E genes from the New York 1999 WNV strain¹² or an expression plasmid encoding the signal sequence of human CD33 linked to the full length WNV NS1 (gift of M. Edeling and D. Fremont, St Louis, Mo.) using FuGENE HD (Roche). Supernatants containing prM-E subviral particles (SVPs) were collected 24 and 48 h after transfection, filtered through a 0.2-μm filter, and stored aliquoted at −80° C. For the capture ELISA, Nunc MaxiSorp polystyrene 96-well plates were coated overnight at 4° C. with mouse E60 mAb⁵ (5 μg/ml) in a pH 9.3 carbonate buffer. Plates were washed three times in enzyme-linked immunosorbent assay (ELISA) wash buffer (PBS with 0.02% Tween 20) and blocked for 1 h at 37° C. with ELISA block buffer (PBS, 2% bovine serum albumin, and 0.02% Tween 20). Supernatants from prM-E plasmid transfected cells were captured on plates coated with E60 for 90 min at room temperature (RT). Subsequently, plates were rinsed five times in wash buffer and then incubated with humanized anti-WNV E16 (1 μg/ml in block buffer) in triplicate for 1 h at RT. Plates were washed five times and then incubated with pre-absorbed biotinylated goat anti-human IgG antibody (1 mg/ml; Jackson Laboratories) for 1 h at RT in blocking buffer. Plates were washed again five times and then sequentially incubated with 2 μg/ml of horseradish peroxidase-conjugated streptavidin (Vector Laboratories) and tetramethylbenzidine substrate (Dako). The reaction was stopped with the addition of 2 N H₂SO₄ to the medium, and emission (450 nm) was read using an iMark microplate reader (Bio-Rad).

Western Blotting.

CRISPR-Cas9 gene-edited 293T cells (10⁶) were lysed directly in 50 ml 5×SDS sample buffer. After heating samples (95° C., 5 min), 10 ml of the preparation was electrophoresed (10% SDS-PAGE) and proteins were transferred to nylon membranes using an iBlot2 Dry Blotting System (Life Technologies). Membranes were blocked with 5% non-fat dry powdered mile and then probed with antibodies. For studies with prM-E and NS1 transfected cells, membranes were probed with anti-E (human WNV E16), anti-NS1 (mouse 8-NS1), and anti-prM (human CR4293¹³), and the relevant secondary antibodies.

293T Cell Viability Assay.

A Vybrant MTT cell viability assay (Life Technologies) was used according to the manufacturer's instructions. Briefly, 10 ml of 12 mM MTT (4,5-dimethylthiazol-2-yl)-2-5-diphenyltetrazolium bromide) was added to 10⁵ 293 T cells (different gene-edited lines, with or without WNV infection) in 100 ml of phenol-red free medium. Cells were incubated for 4h at 37° C., at which time medium was removed and formazan crystals solubilized in 100 ml of DMSO were added for 10 min at 37° C. Liquid was analyzed for absorbance at 540 nm using a Synergy H1 Hybrid Plate Reader (Biotek).

HLA-A2 Surface Protein Expression.

Surface expression of HLA-A2 class I MHC molecules was evaluated using W6/32 (BioLegend), a mouse mAb that recognizes a common determinant on HLA-A, -B, and -C molecules. W6/32 (10 mg/ml) was incubated at 4° C. with individual CRISPR-Cas9 gene-edited cell lines. After incubation with an Alexa Fluor-488 conjugated goat anti-mouse secondary antibody, cells were processed by flow cytometry on a BD FACSArray (Becton Dickinson), and data was processed with FlowJo software (Tree Star, Inc).

Statistical Analysis.

Statistical significance was assigned when P values were <0.05 using GraphPad Prism Version 5.04 (La Jolla, Calif.). Viral antigen staining after expression of sgRNA was analyzed using a one-way ANOVA adjusting for repeated measures with a Dunnett's multiple comparison test or with a Mann-Whitney test depending on the number of comparison groups. Analysis of levels of E protein in the supernatant from CRISPR-Cas9 gene edited cells was analyzed by a one-way ANOVA. Analysis of siRNA in insect and human cells was performed using a Student's T-test.

### REFERENCES FOR THE METHODS

1 Rose, P. P. et al. Natural resistance-associated macrophage protein is a cellular receptor for sindbis virus in both insect and mammalian hosts. Cell Host Microbe 10, 97-104, doi:10.1016/j.chom.2011.06.009 S1931-3128(11)00218-6 [pii] (2011).
2 Brien, J. D., Lazear, H. M. & Diamond, M. S. Propagation, quantification, detection, and storage of West Nile virus. Curr Protoc Microbiol 31, 15D 13 11-15D 13 18, doi:10.1002/9780471729259.mc15d03s31 (2013).
3 Sanjana, N. E., Shalem, O. & Zhang, F. Improved vectors and genome-wide libraries for CRISPR screening. Nat Methods 11, 783-784, doi:10.1038/nmeth.3047. nmeth.3047 [pii] (2014).
4 Fuchs, A., Pinto, A. K., Schwaeble, W. J. & Diamond, M. S. The lectin pathway of complement activation contributes to protection from West Nile virus infection. Virology 412, 101-109, doi:S0042-6822(11)00008-0 [pii]. 10.1016/j.viro1.2011.01.003 (2011).
5 Oliphant, T. et al. Antibody recognition and neutralization determinants on domains I and II of West Nile Virus envelope protein. J Virol 80, 12149-12159 (2006).
6 Boutros, M. et al. Genome-wide RNAi analysis of growth and viability in Drosophila cells. Science 303, 832-835, doi:10.1126/science.1091266. 303/5659/832 [pii] (2004).
7 Hackett, B. A. et al. RNASEK is required for internalization of diverse acid-dependent viruses. Proc Natl Acad Sci USA 112, 7797-7802, doi:10.1073/pnas.1424098112. 1424098112 [pii] (2015).
8 Lin, T. Y. et al. A novel approach for the rapid mutagenesis and directed evolution of the structural genes of west nile virus. J Virol 86, 3501-3512, doi:JVI.06435-11 [pii]. 10.1128/JVI.06435-11 (2012).
9 Pierson, T. C. et al. A rapid and quantitative assay for measuring antibody-mediated neutralization of West Nile virus. Virology 346, 53-65 (2006).
10 Mishin, V. P., Cominelli, F. & Yamshchikov, V. F. A ‘minimal’ approach in design of flavivirus infectious DNA. Virus Res 81, 113-123, doi:S0168170201003719 [pii] (2001).
11 Chung, K. M. et al. Antibodies against West Nile virus non-structural (NS)-1 protein prevent lethal infection through Fc gamma receptor-dependent and independent mechanisms. J Virol 80, 1340-1351 (2006).
12 Vogt, M. R. et al. Human Monoclonal Antibodies Induced by Natural Infection Against West Nile Virus Neutralize at a Post-Attachment Step. J Virol 83, 6494-6507 (2009).
13 Throsby, M. et al. Isolation and characterization of human monoclonal antibodies from individuals infected with West Nile Virus. J Virol 80, 6982-6992 (2006).
14 Youn, S., Cho, H., Fremont, D. H. & Diamond, M. S. A short N-terminal peptide motif on flavivirus nonstructural protein NS1 modulates cellular targeting and immune recognition. J Virol 84, 9516-9532 (2010).
15 Melian, E. B. et al. NS1′ of flaviviruses in the Japanese encephalitis virus serogroup is a product of ribosomal frameshifting and plays a role in viral neuroinvasiveness. J Virol 84, 1641-1647 (2010).

TABLE 1
sgRNA showing enrichment

| Screen 1—Technical repeat 1 | | | | Screen 2—Technical repeat 2 | | | | Screen 2 | | | |
|---|---|---|---|---|---|---|---|---|---|---|---|
| | Un- | WNV | Fold- | | Un- | WNV | Fold- | | Un- | WNV | Fold- |
| gene | infected | infected | enrich | gene | infected | infected | enrich | gene | infected | infected | enrich |
| SPCS3 | 174 | 1424744 | 8188.18 | SPCS3 | 235 | 2542792 | 10820.39 | SEC63 | 158 | 406252 | 2571.22 |
| RAP1GAP | 57 | 377951 | 6630.72 | ZYX | 104 | 199532 | 1918.58 | MARK3 | 244 | 559773 | 2294.15 |
| SEC63 | 200 | 601326 | 3006.63 | PARVG | 41 | 54507 | 1329.44 | SEL1L | 237 | 211140 | 890.89 |
| SLC35G5 | 18 | 32150 | 1786.11 | SEC63 | 264 | 321061 | 1216.14 | EMC4 | 378 | 259120 | 685.50 |
| SEC61A1 | 36 | 34519 | 958.86 | RAPSN | 18 | 13253 | 736.28 | SPCS1 | 114 | 52000 | 456.14 |
| OR6T1 | 88 | 84108 | 955.77 | SEC63 | 280 | 205583 | 734.23 | RPL23A | 65 | 25561 | 393.25 |
| RRAS | 255 | 192540 | 755.06 | SLIT2 | 14 | 9254 | 661.00 | CYP11B2 | 108 | 42208 | 390.81 |
| RAB2B | 105 | 74975 | 714.05 | CYB5A | 159 | 61636 | 387.65 | SEL1L | 195 | 68199 | 349.74 |
| CCSER2 | 22 | 8368 | 380.36 | TPTE2 | 33 | 12402 | 375.82 | KLRK1 | 280 | 93843 | 335.15 |
| SMAD4 | 276 | 90740 | 328.77 | CYP1A2 | 34 | 11091 | 326.21 | SERP1 | 98 | 23999 | 244.89 |
| OSTC | 221 | 72308 | 327.19 | OR52E2 | 218 | 70413 | 323.00 | FIG4 | 7 | 1644 | 234.86 |
| ATOH7 | 81 | 19372 | 239.16 | FBXL20 | 243 | 76780 | 315.97 | FAM212A | 281 | 60908 | 216.75 |
| SPCS3 | 67 | 13809 | 206.10 | SEC61B | 58 | 17688 | 304.97 | PCP2 | 50 | 10099 | 201.98 |
| LRRC37A3 | 348 | 69652 | 200.15 | AP1S3 | 150 | 40256 | 268.37 | SLC26A9 | 116 | 22944 | 197.79 |
| OST4 | 143 | 20481 | 143.22 | CPSF3L | 42 | 9501 | 226.21 | CTSD | 382 | 74112 | 194.01 |
| FEZ2 | 56 | 7261 | 129.66 | hsa-mir-4421 | 16 | 3084 | 192.75 | ESPN | 155 | 29154 | 188.09 |
| SEC63 | 98 | 11879 | 121.21 | STT3A | 149 | 28383 | 190.49 | USB1 | 308 | 49168 | 159.64 |
| DAPL1 | 169 | 18351 | 108.59 | OSTC | 270 | 49888 | 184.77 | FAM151B | 481 | 72317 | 150.35 |
| TBPL1 | 266 | 28757 | 108.11 | ZKSCAN4 | 256 | 46519 | 181.71 | TRERF1 | 296 | 43508 | 146.99 |
| CHCHD7 | 116 | 12437 | 107.22 | HSPA13 | 84 | 12914 | 153.74 | PCDH9 | 263 | 37138 | 141.21 |
| CLEC2D | 119 | 12143 | 102.04 | SEC61B | 242 | 34312 | 141.79 | FBXL20 | 196 | 27535 | 140.48 |
| CHMP1B | 84 | 7678 | 91.40 | PCDHGA6 | 15 | 2099 | 139.93 | GKN2 | 129 | 15933 | 123.51 |
| PRH2 | 283 | 25498 | 90.10 | ALDH16A1 | 51 | 7024 | 137.73 | GORAB | 502 | 60958 | 121.43 |
| hsa-mir-6775 | 23 | 2024 | 88.00 | ASB16 | 170 | 21989 | 129.35 | UNKL | 469 | 53570 | 114.22 |
| SSR3 | 241 | 20343 | 84.41 | KCTD13 | 40 | 5089 | 127.23 | HSPA13 | 120 | 12706 | 105.88 |
| SEC61B | 70 | 5835 | 83.36 | HIST1H2BI | 146 | 16571 | 113.50 | STT3A | 275 | 27910 | 101.49 |
| SEC63 | 297 | 23086 | 77.73 | SCLT1 | 95 | 8745 | 92.05 | ASPSCR1 | 8 | 763 | 95.38 |
| EMC6 | 95 | 7224 | 76.04 | ATG4D | 95 | 8718 | 91.77 | NCOA7 | 46 | 4190 | 91.09 |
| GGTLC1 | 94 | 6416 | 68.26 | APBB3 | 130 | 10341 | 79.55 | C12orf50 | 220 | 16684 | 75.84 |
| RBMX2 | 76 | 4410 | 58.03 | NCAPH2 | 40 | 3006 | 75.15 | KCP | 26 | 1873 | 72.04 |
| C11orf65 | 339 | 17813 | 52.55 | ZSCAN23 | 154 | 10946 | 71.08 | DNAJC25 | 199 | 13734 | 69.02 |
| MORN2 | 714 | 37202 | 52.10 | SEC63 | 109 | 7492 | 68.73 | STT3A | 205 | 13819 | 67.41 |
| TMEM232 | 347 | 13916 | 40.10 | DDIT4 | 139 | 8975 | 64.57 | CD63 | 155 | 9623 | 62.08 |
| TAAR8 | 270 | 10202 | 37.79 | FAM111A | 173 | 11151 | 64.46 | RGMB | 767 | 40968 | 53.41 |
| GZMM | 355 | 13053 | 36.77 | C1orf110 | 226 | 14339 | 63.45 | ADAMTS15 | 165 | 7303 | 44.26 |
| PPP1R13B | 132 | 4596 | 34.82 | DNAJB1 | 242 | 14930 | 61.69 | SSR3 | 476 | 20802 | 43.70 |
| IL26 | 184 | 6211 | 33.76 | KIDINS220 | 170 | 10456 | 61.51 | PASK | 75 | 3030 | 40.40 |
| SERP1 | 216 | 6723 | 31.13 | DCTN5 | 153 | 9297 | 60.76 | USP7 | 180 | 7110 | 39.50 |
| DNAJB14 | 118 | 3593 | 30.45 | STT3A | 96 | 5807 | 60.49 | IFT20 | 240 | 8557 | 35.65 |
| NDST1 | 156 | 4545 | 29.13 | CYP17A1 | 45 | 2667 | 59.27 | PNP | 361 | 11816 | 32.73 |
| hsa-mir-802 | 511 | 14197 | 27.78 | hsa-mir-4442 | 9 | 513 | 57.00 | PHF10 | 210 | 6679 | 31.80 |
| RER1 | 206 | 5723 | 27.78 | TMEM168 | 361 | 20449 | 56.65 | RAB39A | 258 | 7507 | 29.10 |
| CES4A | 108 | 2981 | 27.60 | SERP1 | 204 | 10941 | 53.63 | TRIM21 | 277 | 7957 | 28.73 |
| COA5 | 68 | 1761 | 25.90 | MED26 | 37 | 1943 | 52.51 | SYT10 | 46 | 1278 | 27.78 |
| SEC61B | 225 | 5128 | 22.79 | ABCC9 | 18 | 902 | 50.11 | GGT1 | 74 | 2000 | 27.03 |
| PANX2 | 136 | 2880 | 21.18 | TBK1 | 95 | 4706 | 49.54 | DDR2 | 235 | 6309 | 26.85 |
| RASA1 | 120 | 2527 | 21.06 | OR5L2 | 184 | 8853 | 48.11 | ANKIB1 | 130 | 3145 | 24.19 |
| MOGS | 364 | 7590 | 20.85 | AP4S1 | 136 | 6491 | 47.73 | ESF1 | 62 | 1457 | 23.50 |
| EXO1 | 148 | 2931 | 19.80 | FNBP4 | 57 | 2587 | 45.39 | NT5C2 | 86 | 1960 | 22.79 |
| ANTXR2 | 148 | 2852 | 19.27 | ATP6AP1L | 103 | 4605 | 44.71 | BCAS1 | 142 | 3200 | 22.54 |
| VPS4B | 49 | 935 | 19.08 | TRIM72 | 26 | 1143 | 43.96 | BOP1 | 76 | 1711 | 22.51 |
| hsa-mir-18a | 582 | 11000 | 18.90 | THRB | 143 | 6250 | 43.71 | ZNF75D | 165 | 3601 | 21.82 |
| NPFF | 85 | 1480 | 17.41 | hsa-mir-548as | 41 | 1766 | 43.07 | UBE2J2 | 196 | 4157 | 21.21 |
| hsa-mir-4647 | 147 | 2495 | 16.97 | SYNE2 | 189 | 7808 | 41.31 | MGLL | 19 | 395 | 20.79 |
| MMGT1 | 154 | 2460 | 15.97 | MKL2 | 570 | 23450 | 41.14 | PRDM13 | 110 | 2278 | 20.71 |
| RGMA | 149 | 2339 | 15.70 | hsa-mir-506 | 550 | 22568 | 41.03 | NASP | 93 | 1760 | 18.92 |
| PLCL1 | 185 | 2878 | 15.56 | XIRP1 | 179 | 7183 | 40.13 | AP2B1 | 382 | 7205 | 18.86 |
| GRIP1 | 19 | 295 | 15.53 | PTK2 | 714 | 28185 | 39.47 | SSX1 | 262 | 4750 | 18.13 |
| EMC2 | 29 | 424 | 14.62 | VPRBP | 94 | 3640 | 38.72 | OR2T2 | 575 | 10281 | 17.88 |
| TG | 83 | 1107 | 13.34 | SHC1 | 632 | 23483 | 37.16 | TECPR1 | 76 | 1345 | 17.70 |
| TRAF5 | 186 | 2201 | 11.83 | hsa-mir-4666b | 126 | 4538 | 36.02 | HIST1H2AK | 141 | 2444 | 17.33 |
| ASB5 | 371 | 4082 | 11.00 | hsa-mir-5696 | 186 | 6527 | 35.09 | BEND5 | 592 | 10144 | 17.14 |
| SERP1 | 155 | 1682 | 10.85 | UPK1B | 20 | 677 | 33.85 | EMC3 | 203 | 3365 | 16.58 |
| FES | 278 | 3012 | 10.83 | DKKL1 | 41 | 1382 | 33.71 | ARL6IP5 | 33 | 507 | 15.36 |
| USP20 | 3 | 30 | 10.00 | DDC | 152 | 5116 | 33.66 | TSPYL5 | 85 | 1215 | 14.29 |
| HPRT1 | 104 | 967 | 9.30 | KRTAP13-1 | 39 | 1288 | 33.03 | OR10W1 | 71 | 1007 | 14.18 |
| MUM1L1 | 362 | 3204 | 8.85 | ME2 | 74 | 2408 | 32.54 | FAM120A | 222 | 2919 | 13.15 |
| hsa-mir-132 | 168 | 1415 | 8.42 | BHMT2 | 247 | 7889 | 31.94 | DGKK | 89 | 1115 | 12.53 |
| hsa-mir-4738 | 37 | 310 | 8.38 | hsa-mir-758 | 183 | 5636 | 30.80 | MRPL52 | 223 | 2783 | 12.48 |
| CC2D2B | 158 | 1312 | 8.30 | GLRB | 231 | 7077 | 30.64 | SFMBT2 | 283 | 3516 | 12.42 |
| SYNDIG1L | 117 | 956 | 8.17 | TMEM170B | 29 | 855 | 29.48 | MCCD1 | 656 | 7964 | 12.14 |
| MMGT1 | 41 | 315 | 7.68 | MTM1 | 200 | 5857 | 29.29 | OR4D2 | 175 | 2112 | 12.07 |
| DYNC1LI2 | 118 | 899 | 7.62 | SLC35B4 | 195 | 5710 | 29.28 | RPS25 | 427 | 5084 | 11.91 |
| FAM32A | 704 | 4936 | 7.01 | PQLC1 | 293 | 8496 | 29.00 | SCARF1 | 593 | 6977 | 11.77 |
| FAM73B | 71 | 487 | 6.86 | RLN1 | 205 | 5884 | 28.70 | SCCPDH | 402 | 4717 | 11.73 |
| STT3A | 186 | 1227 | 6.60 | KIAA1239 | 135 | 3837 | 28.42 | KLF3 | 234 | 2726 | 11.65 |
| PVRL3 | 164 | 1077 | 6.57 | PPP1CC | 122 | 3463 | 28.39 | ZC2HC1A | 567 | 6394 | 11.28 |
| EREG | 310 | 2004 | 6.46 | OR13H1 | 208 | 5891 | 28.32 | KRTAP5-6 | 538 | 5769 | 10.72 |
| SCGB2B2 | 126 | 811 | 6.44 | FHIT | 320 | 8861 | 27.69 | ALDOB | 144 | 1500 | 10.42 |
| NPFFR2 | 125 | 767 | 6.14 | GPR63 | 38 | 1032 | 27.16 | EMC4 | 320 | 3262 | 10.19 |
| FAM209B | 267 | 1637 | 6.13 | MC3R | 308 | 7941 | 25.78 | HNRNPF | 100 | 1000 | 10.00 |
| SLC10A5 | 73 | 437 | 5.99 | CDH13 | 47 | 1188 | 25.28 | FAM210A | 70 | 666 | 9.51 |
| BRD3 | 276 | 1605 | 5.82 | HBG1 | 184 | 4633 | 25.18 | GDPD1 | 171 | 1613 | 9.43 |
| GUCA1C | 134 | 774 | 5.78 | BEGAIN | 109 | 2724 | 24.99 | CCR5 | 156 | 1437 | 9.21 |
| TMEM134 | 29 | 167 | 5.76 | OST4 | 162 | 3981 | 24.57 | C20orf85 | 20 | 182 | 9.10 |
| hsa-mir-644a | 105 | 603 | 5.74 | RBM25 | 287 | 7018 | 24.45 | SMARCB1 | 242 | 2162 | 8.93 |
| LY6H | 135 | 770 | 5.70 | DEF6 | 105 | 2555 | 24.33 | KLHL34 | 241 | 2061 | 8.55 |
| MESDC1 | 19 | 104 | 5.47 | STXBP5L | 7 | 169 | 24.14 | PQLC2 | 367 | 3061 | 8.34 |
| DHRS13 | 225 | 1210 | 5.38 | hsa-mir-4723 | 113 | 2657 | 23.51 | LPCAT2 | 240 | 1925 | 8.02 |
| hsa-mir-521-2 | 222 | 1146 | 5.16 | ZNF264 | 95 | 2221 | 23.38 | ZNF347 | 112 | 892 | 7.96 |
| ASB9 | 109 | 532 | 4.88 | ANO10 | 176 | 3831 | 21.77 | NABP1 | 132 | 1043 | 7.90 |
| PREX1 | 171 | 805 | 4.71 | SVEP1 | 182 | 3539 | 19.45 | GPR25 | 83 | 646 | 7.78 |
| MFSD3 | 308 | 1433 | 4.65 | ETS1 | 94 | 1827 | 19.44 | FRS2 | 786 | 5987 | 7.62 |
| ENPP5 | 103 | 455 | 4.42 | KLHL7 | 14 | 268 | 19.14 | RSPO2 | 294 | 2209 | 7.51 |
| ANXA2 | 517 | 2263 | 4.38 | LOC100130451 | 28 | 533 | 19.04 | ARL5A | 180 | 1349 | 7.49 |
| PRRT3 | 66 | 282 | 4.27 | AKAP5 | 295 | 5604 | 19.00 | TCAIM | 287 | 2120 | 7.39 |
| EIF1 | 112 | 471 | 4.21 | BUB3 | 45 | 843 | 18.73 | GBP4 | 396 | 2893 | 7.31 |
| C12orf75 | 34 | 142 | 4.18 | hsa-mir-1-1 | 81 | 1517 | 18.73 | SEC63 | 115 | 827 | 7.19 |
| AFTPH | 68 | 280 | 4.12 | NAA25 | 156 | 2896 | 18.56 | PRAF2 | 96 | 688 | 7.17 |
| POC1B | 466 | 1900 | 4.08 | BEAN1 | 81 | 1502 | 18.54 | CROCC | 208 | 1412 | 6.79 |
| hsa-mir-4780 | 272 | 1067 | 3.92 | SULT4A1 | 77 | 1411 | 18.32 | RBX1 | 206 | 1393 | 6.76 |
| SEC61B | 214 | 837 | 3.91 | LEAP2 | 119 | 2179 | 18.31 | HDLBP | 130 | 878 | 6.75 |
| AR | 210 | 809 | 3.85 | C17orf72 | 94 | 1677 | 17.84 | ADAMTS14 | 276 | 1825 | 6.61 |
| CAV2 | 173 | 655 | 3.79 | MS4A6E | 375 | 6333 | 16.89 | EXT2 | 129 | 820 | 6.36 |
| PLEK2 | 89 | 328 | 3.69 | PTOV1 | 523 | 8787 | 16.80 | CCDC178 | 658 | 4137 | 6.29 |
| PRELID2 | 75 | 274 | 3.65 | FBXO33 | 62 | 1019 | 16.44 | PPRC1 | 303 | 1886 | 6.22 |
| NCKIPSD | 177 | 646 | 3.65 | OTUD6B | 300 | 4832 | 16.11 | FBXL14 | 87 | 536 | 6.16 |
| FCRLA | 293 | 1013 | 3.46 | HIST1H2AB | 48 | 767 | 15.98 | VSIG10L | 117 | 716 | 6.12 |
| CASP8 | 175 | 594 | 3.39 | GUCY2F | 368 | 5713 | 15.52 | DMXL2 | 327 | 1989 | 6.08 |
| SAYSD1 | 201 | 680 | 3.38 | C20orf201 | 40 | 613 | 15.33 | BCAR3 | 133 | 808 | 6.08 |
| ABCA10 | 124 | 415 | 3.35 | DIO3 | 250 | 3783 | 15.13 | PSMG3 | 400 | 2414 | 6.04 |
| LYPLA2 | 148 | 495 | 3.34 | PIGL | 487 | 7186 | 14.76 | PEX11B | 296 | 1773 | 5.99 |
| PHEX | 306 | 1002 | 3.27 | DNAJC24 | 322 | 4650 | 14.44 | TCERG1 | 267 | 1587 | 5.94 |
| ZNF564 | 264 | 855 | 3.24 | CLDND1 | 79 | 1135 | 14.37 | C9orf41 | 224 | 1319 | 5.89 |
| NOA1 | 51 | 161 | 3.16 | MMGT1 | 63 | 905 | 14.37 | USH1G | 209 | 1228 | 5.88 |
| SBDS | 143 | 447 | 3.13 | OSTC | 178 | 2556 | 14.36 | SFT2D3 | 161 | 940 | 5.84 |
| LRRC40 | 126 | 386 | 3.06 | STAB2 | 185 | 2594 | 14.02 | MYO3B | 183 | 1067 | 5.83 |
| BRI3BP | 122 | 373 | 3.06 | CLVS2 | 46 | 644 | 14.00 | MEIG1 | 585 | 3388 | 5.79 |
| MPND | 767 | 2338 | 3.05 | GALNT1 | 29 | 404 | 13.93 | PDCD6 | 119 | 680 | 5.71 |
| RLF | 317 | 961 | 3.03 | SAMD9 | 462 | 6398 | 13.85 | BTN2A2 | 560 | 3158 | 5.64 |
| hsa-mir-3166 | 233 | 699 | 3.00 | INS | 200 | 2740 | 13.70 | NVL | 70 | 389 | 5.56 |
| TMEM203 | 203 | 594 | 2.93 | HIST1H2AA | 49 | 667 | 13.61 | C2orf40 | 85 | 468 | 5.51 |
| C4orf48 | 144 | 413 | 2.87 | SERP1 | 157 | 2133 | 13.59 | ADIPOR2 | 262 | 1430 | 5.46 |
| CNOT4 | 178 | 509 | 2.86 | P2RY14 | 83 | 1100 | 13.25 | WBSCR28 | 232 | 1254 | 5.41 |
| THAP8 | 178 | 506 | 2.84 | VIM | 195 | 2534 | 12.99 | AKAP13 | 411 | 2217 | 5.39 |
| ERMP1 | 272 | 770 | 2.83 | CTPS1 | 103 | 1331 | 12.92 | ARF1 | 184 | 989 | 5.38 |
| MCCD1 | 152 | 428 | 2.82 | CST3 | 32 | 412 | 12.88 | HIST1H2BL | 183 | 954 | 5.21 |
| HSPA13 | 103 | 285 | 2.77 | SLC25A14 | 37 | 474 | 12.81 | LCN6 | 221 | 1146 | 5.19 |
| hsa-mir-5696 | 334 | 867 | 2.60 | ZFP36L1 | 216 | 2761 | 12.78 | FGR | 116 | 594 | 5.12 |
| hsa-mir-1289-2 | 101 | 254 | 2.51 | SLU7 | 186 | 2375 | 12.77 | EIF4A3 | 69 | 352 | 5.10 |
| ELANE | 356 | 879 | 2.47 | GYPB | 394 | 5029 | 12.76 | NT5DC3 | 271 | 1372 | 5.06 |
| FAM210A | 192 | 466 | 2.43 | PSORS1C1 | 438 | 5585 | 12.75 | MYEOV2 | 94 | 474 | 5.04 |
| HSPA13 | 137 | 331 | 2.42 | STX8 | 64 | 810 | 12.66 | RBP5 | 28 | 139 | 4.96 |
| BRIP1 | 456 | 1099 | 2.41 | STT3A | 135 | 1704 | 12.62 | LIN7A | 484 | 2357 | 4.87 |
| FAM181B | 284 | 683 | 2.40 | RAP1GAP2 | 99 | 1247 | 12.60 | DOK1 | 388 | 1886 | 4.86 |
| XKRX | 165 | 394 | 2.39 | GREM2 | 200 | 2508 | 12.54 | RUNDC3B | 76 | 364 | 4.79 |
| MRPS7 | 43 | 100 | 2.33 | FGFR2 | 187 | 2330 | 12.46 | NLRP2 | 95 | 442 | 4.65 |
| FBP1 | 100 | 229 | 2.29 | SEC11A | 474 | 5892 | 12.43 | SLC25A42 | 237 | 1101 | 4.65 |
| SDPR | 618 | 1414 | 2.29 | hsa-mir-1976 | 176 | 2159 | 12.27 | NARF | 589 | 2680 | 4.55 |
| PTPRU | 320 | 682 | 2.13 | SNRNP200 | 97 | 1189 | 12.26 | ASCC2 | 353 | 1604 | 4.54 |
| STT3A | 127 | 266 | 2.09 | hsa-mir-4683 | 297 | 3554 | 11.97 | EIF4ENIF1 | 314 | 1416 | 4.51 |
| OR2G6 | 233 | 479 | 2.06 | RAB37 | 128 | 1527 | 11.93 | EMC3 | 490 | 2188 | 4.47 |
| YLPM1 | 132 | 269 | 2.04 | NTRK3 | 61 | 727 | 11.92 | GNG8 | 275 | 1212 | 4.41 |
| SPCS1 | 14 | 28 | 2.00 | SHROOM2 | 271 | 3198 | 11.80 | MCM8 | 185 | 812 | 4.39 |
| CLCC1 | 38 | 75 | 1.97 | ATF2 | 214 | 2480 | 11.59 | AKIP1 | 132 | 577 | 4.37 |
| RPL5 | 14 | 27 | 1.93 | RGS22 | 106 | 1227 | 11.58 | FAM72D | 396 | 1676 | 4.23 |
| CLCC1 | 286 | 547 | 1.91 | LSMEM2 | 84 | 957 | 11.39 | ABCC4 | 215 | 896 | 4.17 |
| MARVELD2 | 44 | 84 | 1.91 | KLHL28 | 56 | 626 | 11.18 | HTR1E | 242 | 1998 | 7.06 |
| CD163L1 | 399 | 761 | 1.91 | ZNF620 | 67 | 747 | 11.15 | HIF3A | 114 | 468 | 4.11 |
| APCS | 112 | 213 | 1.90 | AK9 | 126 | 1386 | 11.00 | FBXO9 | 565 | 2309 | 4.09 |
| hsa-mir-323a | 476 | 874 | 1.84 | MICALL1 | 128 | 1407 | 10.99 | FUT2 | 610 | 2399 | 3.93 |
| ACSM4 | 158 | 284 | 1.80 | SENP5 | 227 | 2486 | 10.95 | HIST2H2BF | 273 | 1058 | 3.88 |
| GPSM3 | 190 | 340 | 1.79 | SHQ1 | 82 | 865 | 10.55 | TMEM253 | 42 | 162 | 3.86 |
| PRR14L | 302 | 535 | 1.77 | C4orf21 | 322 | 3348 | 10.40 | INPP5B | 249 | 957 | 3.84 |
| XYLT1 | 161 | 284 | 1.76 | POLR2A | 666 | 6877 | 10.33 | C16orf97 | 485 | 1824 | 3.76 |
| ALDH1A2 | 119 | 209 | 1.76 | DFNB31 | 354 | 3619 | 10.22 | AK8 | 99 | 369 | 3.73 |
| hsa-mir-4804 | 126 | 217 | 1.72 | BEX5 | 9 | 90 | 10.00 | MAP9 | 229 | 848 | 3.70 |
| hsa-mir-9-3 | 124 | 211 | 1.70 | AMOTL2 | 260 | 2588 | 9.95 | MICU3 | 117 | 432 | 3.69 |
| hsa-mir-137 | 20 | 34 | 1.70 | hsa-mir-103a-1 | 284 | 2773 | 9.76 | CINP | 225 | 829 | 3.68 |
| SERPINI2 | 86 | 146 | 1.70 | MS4A4A | 97 | 918 | 9.46 | CYP2B6 | 174 | 633 | 3.64 |
| BAG5 | 225 | 366 | 1.63 | SP5 | 357 | 3342 | 9.36 | CCDC179 | 448 | 1625 | 3.63 |
| GSC | 153 | 248 | 1.62 | hsa-mir-4295 | 57 | 532 | 9.33 | TMEM200B | 64 | 227 | 3.55 |
| KIF2B | 48 | 76 | 1.58 | hsa-mir-146b | 172 | 1587 | 9.23 | CLOCK | 143 | 503 | 3.52 |
| SAMD5 | 67 | 105 | 1.57 | NFIB | 134 | 1233 | 9.20 | SNAPC1 | 111 | 388 | 3.50 |
| E2F6 | 206 | 314 | 1.52 | MAN1B1 | 78 | 717 | 9.19 | DCHS1 | 226 | 788 | 3.49 |
| ZNF18 | 313 | 471 | 1.50 | hsa-mir-4440 | 48 | 440 | 9.17 | HAP1 | 108 | 373 | 3.45 |
| LIMK2 | 448 | 665 | 1.48 | hsa-mir-196a-2 | 285 | 2603 | 9.13 | PDE11A | 468 | 1612 | 3.44 |
| ACAD10 | 77 | 114 | 1.48 | DTX3 | 439 | 3966 | 9.03 | PNO1 | 164 | 558 | 3.40 |
| GIF | 385 | 557 | 1.45 | NEGR1 | 78 | 698 | 8.95 | CLEC11A | 414 | 1408 | 3.40 |
| FAM20B | 121 | 175 | 1.45 | SEC62 | 178 | 1573 | 8.84 | RAB11FIP5 | 276 | 935 | 3.39 |
| C22orf24 | 181 | 258 | 1.43 | RD3 | 236 | 2084 | 8.83 | GSE1 | 154 | 506 | 3.29 |
| C1orf168 | 170 | 242 | 1.42 | AP1B1 | 141 | 1243 | 8.82 | GCC1 | 133 | 436 | 3.28 |
| PSG8 | 217 | 306 | 1.41 | GGT6 | 64 | 558 | 8.72 | RABEP2 | 127 | 407 | 3.20 |
| TECRL | 105 | 147 | 1.40 | hsa-mir-1825 | 93 | 805 | 8.66 | TMEM14A | 254 | 805 | 3.17 |
| EMC6 | 95 | 133 | 1.40 | SMARCE1 | 183 | 1569 | 8.57 | EPAS1 | 328 | 1028 | 3.13 |
| hsa-mir-759 | 96 | 133 | 1.39 | METTL20 | 174 | 1485 | 8.53 | SPINK4 | 692 | 2125 | 3.07 |
| PLA2G10 | 283 | 385 | 1.36 | EFCAB3 | 292 | 2490 | 8.53 | STAU2 | 328 | 1007 | 3.07 |
| hsa-mir-140 | 272 | 370 | 1.36 | SET | 72 | 611 | 8.49 | PDE10A | 144 | 441 | 3.06 |
| ZFYVE16 | 234 | 316 | 1.35 | NDUFAF1 | 116 | 974 | 8.40 | TMEM100 | 303 | 902 | 2.98 |
| XDH | 89 | 119 | 1.34 | OR5J2 | 187 | 1565 | 8.37 | IFRD2 | 127 | 375 | 2.95 |
| SENP8 | 61 | 81 | 1.33 | ZCCHC10 | 165 | 1380 | 8.36 | SHC1 | 150 | 440 | 2.93 |
| INSIG2 | 255 | 338 | 1.33 | HAUS2 | 230 | 1921 | 8.35 | FADS3 | 42 | 123 | 2.93 |
| S100A11 | 156 | 204 | 1.31 | BRWD3 | 125 | 1035 | 8.28 | APCDD1L | 401 | 1167 | 2.91 |
| CDKN2C | 452 | 583 | 1.29 | PLCH1 | 119 | 985 | 8.28 | WFDC6 | 88 | 253 | 2.88 |
| PXDC1 | 324 | 415 | 1.28 | EP400 | 149 | 1224 | 8.21 | C1QL1 | 277 | 795 | 2.87 |
| VWA3B | 209 | 266 | 1.27 | FYN | 285 | 2340 | 8.21 | EPHA3 | 154 | 428 | 2.78 |
| TTC18 | 111 | 138 | 1.24 | hsa-mir-6131 | 171 | 1387 | 8.11 | SLITRK4 | 305 | 847 | 2.78 |
| NTM | 873 | 1080 | 1.24 | SMIM17 | 117 | 942 | 8.05 | BRD3 | 228 | 626 | 2.75 |
| SYVN1 | 192 | 226 | 1.18 | FRMD1 | 98 | 781 | 7.97 | GPIHBP1 | 76 | 206 | 2.71 |
| PHTF2 | 215 | 253 | 1.18 | IRF7 | 154 | 1221 | 7.93 | MRPL2 | 122 | 327 | 2.68 |
| GLIPR1L2 | 97 | 112 | 1.15 | NCMAP | 123 | 974 | 7.92 | COX19 | 154 | 411 | 2.67 |
| SDSL | 69 | 79 | 1.14 | hsa-mir-539 | 73 | 577 | 7.90 | OAS3 | 143 | 381 | 2.66 |
| TIMP4 | 113 | 127 | 1.12 | LYN | 253 | 1988 | 7.86 | STT3A | 127 | 326 | 2.57 |
| EMC3 | 80 | 89 | 1.11 | MBNL3 | 91 | 708 | 7.78 | KDM5D | 877 | 2220 | 2.53 |
| IL19 | 117 | 129 | 1.10 | FFAR1 | 126 | 969 | 7.69 | TMCO4 | 261 | 642 | 2.46 |
| GDF1 | 148 | 163 | 1.10 | TBCEL | 347 | 2662 | 7.67 | PFKM | 156 | 381 | 2.44 |
| ZNF653 | 249 | 274 | 1.10 | DST | 350 | 2670 | 7.63 | ACIN1 | 178 | 433 | 2.43 |
| PRSS45 | 54 | 59 | 1.09 | TSSK6 | 135 | 1019 | 7.55 | SMTN | 152 | 369 | 2.43 |
| EMC6 | 141 | 153 | 1.09 | PCGF1 | 142 | 1070 | 7.54 | HTR4 | 206 | 492 | 2.39 |
| ZNF670 | 331 | 354 | 1.07 | AK3 | 534 | 3996 | 7.48 | CHIC2 | 262 | 624 | 2.38 |
| FUCA2 | 385 | 410 | 1.06 | VEZT | 122 | 894 | 7.33 | ZCCHC24 | 16 | 38 | 2.38 |
| ADAP1 | 109 | 113 | 1.04 | ADAM20 | 51 | 363 | 7.12 | CTSV | 71 | 168 | 2.37 |
| OTUD4 | 67 | 69 | 1.03 | ANK2 | 389 | 2738 | 7.04 | SLC20A1 | 279 | 643 | 2.30 |
| KCTD16 | 468 | 481 | 1.03 | RAB30 | 114 | 801 | 7.03 | ABCC1 | 285 | 654 | 2.29 |
| WNT5B | 208 | 212 | 1.02 | TMC7 | 411 | 2865 | 6.97 | SLC8A2 | 219 | 500 | 2.28 |
| FXYD3 | 82 | 82 | 1.00 | OR4D5 | 28 | 194 | 6.93 | DGKZ | 378 | 863 | 2.28 |
| SRP72 | 61 | 61 | 1.00 | STAT4 | 241 | 1658 | 6.88 | HOXD8 | 225 | 507 | 2.25 |
| LYNX1 | 192 | 185 | 0.96 | CAMSAP1 | 269 | 1842 | 6.85 | HAS2 | 141 | 316 | 2.24 |
| MTMR2 | 394 | 379 | 0.96 | TDRD7 | 292 | 1993 | 6.83 | CCDC8 | 283 | 630 | 2.23 |
| hsa-mir-891b | 365 | 348 | 0.95 | hsa-mir-500a | 454 | 3074 | 6.77 | KIF25 | 289 | 638 | 2.21 |
| MED10 | 77 | 72 | 0.94 | CDRT1 | 86 | 581 | 6.76 | TRANK1 | 557 | 1223 | 2.20 |
| BPIFB4 | 88 | 82 | 0.93 | RNF112 | 233 | 1565 | 6.72 | LDLRAD4 | 590 | 1277 | 2.16 |
| ARHGAP18 | 169 | 157 | 0.93 | COPS5 | 348 | 2336 | 6.71 | ALB | 153 | 330 | 2.16 |
| GABRG3 | 156 | 144 | 0.92 | NOC4L | 83 | 539 | 6.49 | CARNS1 | 110 | 236 | 2.15 |
| hsa-mir-302e | 184 | 168 | 0.91 | ATL3 | 267 | 1690 | 6.33 | ZIC2 | 331 | 705 | 2.13 |
| RHEB | 211 | 189 | 0.90 | ZNF844 | 107 | 676 | 6.32 | RBM25 | 98 | 207 | 2.11 |
| SLC28A3 | 425 | 378 | 0.89 | CEACAM21 | 184 | 1158 | 6.29 | NMNAT2 | 263 | 542 | 2.06 |
| DCTD | 188 | 166 | 0.88 | RAPGEF4 | 88 | 545 | 6.19 | OR51M1 | 259 | 532 | 2.05 |
| GSTT1 | 126 | 110 | 0.87 | DCST2 | 126 | 776 | 6.16 | DENND3 | 143 | 291 | 2.03 |
| INPP4A | 228 | 196 | 0.86 | EHBP1 | 137 | 840 | 6.13 | C9orf57 | 176 | 357 | 2.03 |
| KIF4B | 60 | 50 | 0.83 | INPP5F | 91 | 551 | 6.05 | CCL26 | 201 | 407 | 2.02 |
| KRTAP5-5 | 243 | 200 | 0.82 | hsa-mir-1260a | 140 | 833 | 5.95 | COX6C | 234 | 470 | 2.01 |
| GLG1 | 176 | 144 | 0.82 | EPHX3 | 283 | 1669 | 5.90 | ITFG3 | 180 | 358 | 1.99 |
| hsa-mir-521-1 | 436 | 356 | 0.82 | ZNF488 | 115 | 674 | 5.86 | PAX5 | 262 | 520 | 1.98 |
| TMEM151B | 304 | 243 | 0.80 | HSPA12B | 88 | 515 | 5.85 | COQ10A | 190 | 377 | 1.98 |
| HBD | 389 | 310 | 0.80 | TRIM69 | 119 | 687 | 5.77 | GPR161 | 293 | 581 | 1.98 |
| ART1 | 87 | 68 | 0.78 | SLITRK6 | 318 | 1820 | 5.72 | KIAA0930 | 213 | 414 | 1.94 |
| DHX35 | 31 | 24 | 0.77 | CAPNS1 | 220 | 1249 | 5.68 | MAP2K5 | 402 | 779 | 1.94 |
| hsa-mir-4803 | 75 | 58 | 0.77 | hsa-mir-31 | 115 | 643 | 5.59 | C11orf65 | 218 | 422 | 1.94 |
| DENND3 | 121 | 92 | 0.76 | HBM | 264 | 1475 | 5.59 | GFRA4 | 804 | 1550 | 1.93 |
| CYP2F1 | 175 | 132 | 0.75 | WDR96 | 70 | 390 | 5.57 | PPA1 | 142 | 273 | 1.92 |
| ELP4 | 290 | 217 | 0.75 | SEMA4A | 65 | 362 | 5.57 | LOC100287177 | 243 | 465 | 1.91 |
| GTF2B | 223 | 166 | 0.74 | hsa-mir-891b | 427 | 2376 | 5.56 | EML6 | 223 | 423 | 1.90 |
| TNFAIP8L2 | 166 | 123 | 0.74 | MAGEA1 | 100 | 552 | 5.52 | FAM214B | 241 | 447 | 1.85 |
| YPEL4 | 403 | 293 | 0.73 | CCDC25 | 403 | 2201 | 5.46 | ACSS1 | 189 | 344 | 1.82 |
| HES7 | 29 | 21 | 0.72 | SLC30A10 | 147 | 796 | 5.41 | PVRL3 | 195 | 349 | 1.79 |
| EOMES | 46 | 33 | 0.72 | ZC3H4 | 126 | 672 | 5.33 | PCDHA13 | 146 | 261 | 1.79 |
| GNPDA1 | 380 | 266 | 0.70 | OR56B1 | 132 | 698 | 5.29 | CHM | 56 | 100 | 1.79 |
| BCHE | 48 | 33 | 0.69 | hsa-mir-7515 | 7 | 37 | 5.29 | TADA2A | 68 | 121 | 1.78 |
| ASPM | 382 | 260 | 0.68 | TRAF1 | 149 | 787 | 5.28 | VPS37D | 157 | 279 | 1.78 |
| GPR68 | 73 | 49 | 0.67 | ERVFRD-1 | 255 | 1346 | 5.28 | LTA4H | 526 | 934 | 1.78 |
| TMEM72 | 229 | 153 | 0.67 | CD1D | 171 | 877 | 5.13 | OR51E1 | 307 | 545 | 1.78 |
| EMC3 | 696 | 450 | 0.65 | C1orf50 | 12 | 61 | 5.08 | DTHD1 | 88 | 156 | 1.77 |
| ZYX | 129 | 83 | 0.64 | ERVV-1 | 65 | 329 | 5.06 | BLVRB | 214 | 374 | 1.75 |
| XPO4 | 266 | 167 | 0.63 | SLC26A6 | 185 | 934 | 5.05 | KLB | 285 | 493 | 1.73 |
| OR1G1 | 74 | 46 | 0.62 | SOD3 | 198 | 989 | 4.99 | CNTROB | 327 | 564 | 1.72 |
| RAVER1 | 274 | 170 | 0.62 | PER1 | 253 | 1236 | 4.89 | PFN4 | 449 | 766 | 1.71 |
| SETD9 | 274 | 170 | 0.62 | UBL7 | 240 | 1170 | 4.88 | ST8SIA6 | 500 | 845 | 1.69 |
| TMEM165 | 420 | 259 | 0.62 | C17orf85 | 222 | 1070 | 4.82 | WNK3 | 241 | 405 | 1.68 |
| TBATA | 69 | 42 | 0.61 | PLCXD3 | 166 | 798 | 4.81 | ZNF543 | 234 | 387 | 1.65 |
| DENND5B | 267 | 162 | 0.61 | FAM109A | 228 | 1095 | 4.80 | SH3GL1 | 293 | 482 | 1.65 |
| ERLEC1 | 237 | 141 | 0.59 | DCST1 | 151 | 721 | 4.77 | CPXM2 | 748 | 1228 | 1.64 |
| SPTA1 | 341 | 202 | 0.59 | SERPINC1 | 236 | 1124 | 4.76 | ZNF706 | 523 | 850 | 1.63 |
| CHIC2 | 491 | 290 | 0.59 | SOS2 | 140 | 664 | 4.74 | SFRP2 | 93 | 148 | 1.59 |
| INTS2 | 78 | 46 | 0.59 | WBSCR16 | 229 | 1083 | 4.73 | COX7A1 | 335 | 532 | 1.59 |
| OR5L1 | 212 | 125 | 0.59 | C10orf25 | 121 | 572 | 4.73 | NR3C1 | 225 | 356 | 1.58 |
| NAT10 | 232 | 136 | 0.59 | SEC24B | 479 | 2262 | 4.72 | RCBTB2 | 201 | 318 | 1.58 |
| FAM19A4 | 95 | 53 | 0.56 | ARHGAP8 | 199 | 930 | 4.67 | TNRC6C | 84 | 132 | 1.57 |
| GLIS3 | 85 | 47 | 0.55 | TRPV6 | 154 | 715 | 4.64 | LACTB2 | 331 | 514 | 1.55 |
| TNNI2 | 152 | 84 | 0.55 | EFHC2 | 197 | 914 | 4.64 | NUCB1 | 667 | 1035 | 1.55 |
| TTBK2 | 241 | 132 | 0.55 | CPSF6 | 95 | 428 | 4.51 | YIPF4 | 200 | 308 | 1.54 |
| KRT8 | 165 | 90 | 0.55 | CCNL1 | 16 | 71 | 4.44 | HSPA12B | 41 | 63 | 1.54 |
| hsa-mir-1302-5 | 410 | 223 | 0.54 | hsa-mir-4675 | 193 | 855 | 4.43 | OTOP2 | 117 | 179 | 1.53 |
| PRKCG | 70 | 38 | 0.54 | TTC16 | 138 | 600 | 4.35 | OR8D1 | 142 | 217 | 1.53 |
| GCG | 405 | 218 | 0.54 | UBE2G2 | 376 | 1616 | 4.30 | GLI4 | 207 | 310 | 1.50 |
| HIST1H3F | 73 | 39 | 0.53 | IDO1 | 190 | 795 | 4.18 | ZNF490 | 147 | 220 | 1.50 |
| hsa-mir-578 | 86 | 45 | 0.52 | PCMTD1 | 346 | 1446 | 4.18 | AUP1 | 59 | 88 | 1.49 |
| FNDC3B | 267 | 138 | 0.52 | AUP1 | 259 | 1080 | 4.17 | PDE7B | 1039 | 1542 | 1.48 |
| CDKN2D | 327 | 169 | 0.52 | EPOR | 61 | 253 | 4.15 | MAN1B1 | 29 | 43 | 1.48 |
| hsa-mir-96 | 273 | 140 | 0.51 | WDFY2 | 237 | 982 | 4.14 | OR2T4 | 253 | 374 | 1.48 |
| DDA1 | 181 | 92 | 0.51 | NUP107 | 199 | 824 | 4.14 | PITPNC1 | 143 | 211 | 1.48 |
| FAM217A | 28 | 14 | 0.50 | SLC39A2 | 201 | 832 | 4.14 | CCDC47 | 120 | 177 | 1.48 |
| CCDC167 | 8 | 4 | 0.50 | CTIF | 157 | 637 | 4.06 | C1orf112 | 222 | 327 | 1.47 |
| CNBD1 | 155 | 77 | 0.50 | CRISP2 | 30 | 121 | 4.03 | FUCA1 | 797 | 1169 | 1.47 |
| FAM180A | 222 | 110 | 0.50 | OR5P3 | 648 | 2605 | 4.02 | ALDH4A1 | 172 | 245 | 1.42 |
| STEAP4 | 145 | 69 | 0.48 | C1QL3 | 155 | 621 | 4.01 | ODF3L2 | 224 | 315 | 1.41 |
| CCR6 | 358 | 169 | 0.47 | LAMA1 | 299 | 1195 | 4.00 | IFNGR2 | 566 | 794 | 1.40 |
| OLIG2 | 114 | 53 | 0.46 | hsa-mir-548ai | 111 | 442 | 3.98 | CHCHD4 | 233 | 325 | 1.39 |
| VAPA | 91 | 42 | 0.46 | SPANXF1 | 896 | 3526 | 3.94 | SLC35D1 | 236 | 329 | 1.39 |
| C15orf62 | 128 | 59 | 0.46 | BRWD1 | 107 | 419 | 3.92 | FCRLB | 184 | 252 | 1.37 |
| ZBTB5 | 204 | 94 | 0.46 | hsa-mir-4660 | 130 | 505 | 3.88 | HIVEP2 | 46 | 63 | 1.37 |
| AMOT | 163 | 75 | 0.46 | GK2 | 244 | 943 | 3.86 | TTC29 | 336 | 455 | 1.35 |
| EMC2 | 64 | 29 | 0.45 | WWC3 | 186 | 709 | 3.81 | FAM26D | 89 | 119 | 1.34 |
| PARVG | 31 | 14 | 0.45 | TNFAIP8 | 73 | 277 | 3.79 | PKN1 | 122 | 163 | 1.34 |
| hsa-mir-130b | 60 | 27 | 0.45 | RASSF9 | 159 | 603 | 3.79 | DEDD2 | 231 | 308 | 1.33 |
| CCDC106 | 147 | 66 | 0.45 | PCDHB14 | 126 | 474 | 3.76 | C12orf54 | 879 | 1164 | 1.32 |
| DCAF4L2 | 148 | 66 | 0.45 | ABLIM3 | 355 | 1333 | 3.75 | RBBP6 | 38 | 50 | 1.32 |
| hsa-mir-2681 | 275 | 122 | 0.44 | FAM71F2 | 520 | 1936 | 3.72 | P2RY12 | 121 | 157 | 1.30 |
| IL22RA1 | 46 | 20 | 0.43 | HDAC10 | 136 | 505 | 3.71 | HBB | 169 | 219 | 1.30 |
| KIAA1217 | 148 | 64 | 0.43 | POU5F2 | 36 | 130 | 3.61 | NADSYN1 | 62 | 80 | 1.29 |
| hsa-mir-4286 | 132 | 57 | 0.43 | DOCK2 | 677 | 2388 | 3.53 | TACO1 | 273 | 345 | 1.26 |
| RBMX | 106 | 44 | 0.42 | TPR | 78 | 275 | 3.53 | TYW1 | 587 | 738 | 1.26 |
| LUZP2 | 122 | 50 | 0.41 | TAS2R39 | 151 | 529 | 3.50 | CTSF | 105 | 131 | 1.25 |
| NXPH4 | 108 | 44 | 0.41 | C15orf39 | 120 | 417 | 3.48 | JRKL | 206 | 256 | 1.24 |
| KIAA0907 | 370 | 149 | 0.40 | TAS2R46 | 696 | 2409 | 3.46 | BTBD16 | 100 | 124 | 1.24 |
| SLC4A5 | 243 | 97 | 0.40 | ITGA7 | 341 | 1176 | 3.45 | PDE8A | 192 | 238 | 1.24 |
| ZNF93 | 254 | 100 | 0.39 | C4orf26 | 175 | 594 | 3.39 | XPO7 | 682 | 844 | 1.24 |
| AP4M1 | 379 | 149 | 0.39 | hsa-mir-6836 | 84 | 285 | 3.39 | ZNF560 | 19 | 23 | 1.21 |
| GCSAML | 51 | 20 | 0.39 | SYT15 | 55 | 186 | 3.38 | CBLN4 | 232 | 277 | 1.19 |
| VASH2 | 291 | 111 | 0.38 | hsa-mir-1208 | 124 | 413 | 3.33 | POP7 | 53 | 63 | 1.19 |
| HAP1 | 250 | 94 | 0.38 | hsa-mir-4692 | 70 | 233 | 3.33 | MPZL3 | 95 | 112 | 1.18 |
| TRNT1 | 104 | 39 | 0.38 | hsa-mir-4792 | 43 | 143 | 3.33 | SDR9C7 | 175 | 206 | 1.18 |
| hsa-mir-378f | 235 | 88 | 0.37 | OR51Q1 | 31 | 103 | 3.32 | OR14J1 | 501 | 587 | 1.17 |
| SOX30 | 206 | 77 | 0.37 | HGF | 284 | 939 | 3.31 | ZNF407 | 120 | 140 | 1.17 |
| GUSB | 163 | 60 | 0.37 | OTUB1 | 376 | 1232 | 3.28 | TMEM194B | 65 | 75 | 1.15 |
| MAPRE3 | 300 | 110 | 0.37 | UBFD1 | 282 | 914 | 3.24 | OR10R2 | 549 | 633 | 1.15 |
| STAG3 | 267 | 97 | 0.36 | SOSTDC1 | 235 | 727 | 3.09 | SHISA2 | 300 | 345 | 1.15 |
| R3HDM2 | 205 | 74 | 0.36 | UBL4A | 692 | 2139 | 3.09 | CENPC1 | 327 | 374 | 1.14 |
| KRTAP9-6 | 347 | 124 | 0.36 | DEXI | 327 | 1009 | 3.09 | ZNF529 | 154 | 174 | 1.13 |
| hsa-mir-2861 | 79 | 28 | 0.35 | FLVCR1 | 45 | 138 | 3.07 | KRT6B | 177 | 199 | 1.12 |
| SNRPN | 289 | 102 | 0.35 | HMGB2 | 185 | 560 | 3.03 | FAM151B | 280 | 314 | 1.12 |
| LEO1 | 153 | 54 | 0.35 | hsa-mir-3187 | 383 | 1151 | 3.01 | HPDL | 203 | 226 | 1.11 |
| IMPDH2 | 88 | 31 | 0.35 | COL25A1 | 326 | 978 | 3.00 | LSM3 | 54 | 60 | 1.11 |
| hsa-mir-200b | 216 | 76 | 0.35 | SNX7 | 101 | 303 | 3.00 | CSF2 | 224 | 248 | 1.11 |
| HLA-DRB5 | 172 | 60 | 0.35 | GPR61 | 55 | 165 | 3.00 | PCDH11Y | 182 | 201 | 1.10 |
| hsa-mir-556 | 73 | 25 | 0.34 | PSME1 | 167 | 499 | 2.99 | UTS2 | 195 | 215 | 1.10 |
| SCML2 | 280 | 95 | 0.34 | GZMB | 403 | 1204 | 2.99 | NFATC2 | 427 | 470 | 1.10 |
| GABRA3 | 118 | 40 | 0.34 | FAM3D | 66 | 196 | 2.97 | ADCYAP1R1 | 191 | 210 | 1.10 |
| IL9 | 210 | 70 | 0.33 | ZNF329 | 148 | 439 | 2.97 | SLAMF9 | 119 | 130 | 1.09 |
| VSNL1 | 46 | 15 | 0.33 | EXOC7 | 52 | 151 | 2.90 | SETD7 | 273 | 297 | 1.09 |
| GNAL | 120 | 39 | 0.33 | SGK3 | 123 | 355 | 2.89 | MXRA7 | 585 | 636 | 1.09 |
| TXN2 | 238 | 77 | 0.32 | hsa-mir-8057 | 34 | 98 | 2.88 | SLC14A1 | 627 | 677 | 1.08 |
| C5orf20 | 598 | 192 | 0.32 | MNS1 | 53 | 152 | 2.87 | MSC | 293 | 313 | 1.07 |
| RSAD2 | 202 | 63 | 0.31 | MANBA | 339 | 972 | 2.87 | LRRC29 | 91 | 97 | 1.07 |
| ALOXE3 | 209 | 65 | 0.31 | PLEKHO2 | 133 | 377 | 2.83 | TEK | 92 | 98 | 1.07 |
| FAM24A | 164 | 51 | 0.31 | MLL5 | 157 | 445 | 2.83 | SOST | 71 | 72 | 1.01 |
| SYPL1 | 342 | 106 | 0.31 | OR10X1 | 513 | 1449 | 2.82 | SCGB3A2 | 443 | 446 | 1.01 |
| RBP5 | 459 | 141 | 0.31 | RILPL2 | 45 | 127 | 2.82 | TAS2R3 | 641 | 643 | 1.00 |
| ARSB | 212 | 65 | 0.31 | BBS10 | 44 | 124 | 2.82 | SLC24A4 | 434 | 435 | 1.00 |
| TNFSF12 | 92 | 28 | 0.30 | WFDC8 | 258 | 726 | 2.81 | MYOZ3 | 94 | 94 | 1.00 |
| hsa-mir-4733 | 211 | 64 | 0.30 | MRPL46 | 146 | 409 | 2.80 | APLNR | 5 | 5 | 1.00 |
| EFCAB4B | 63 | 19 | 0.30 | ZNF597 | 584 | 1633 | 2.80 | CASP3 | 750 | 749 | 1.00 |
| hsa-mir-107 | 355 | 105 | 0.30 | AREG | 166 | 463 | 2.79 | FAM204A | 76 | 75 | 0.99 |
| FZD1 | 44 | 13 | 0.30 | FSCB | 272 | 758 | 2.79 | NUCKS1 | 478 | 468 | 0.98 |
| RILPL1 | 184 | 54 | 0.29 | CDKN1B | 269 | 741 | 2.75 | PSAP | 270 | 263 | 0.97 |
| COG4 | 103 | 30 | 0.29 | RAP1GAP | 43 | 118 | 2.74 | BARHL1 | 152 | 147 | 0.97 |
| SLC24A2 | 171 | 49 | 0.29 | KCNN3 | 131 | 357 | 2.73 | RESP18 | 449 | 430 | 0.96 |
| OR5H15 | 161 | 46 | 0.29 | AEBP2 | 139 | 377 | 2.71 | MMGT1 | 298 | 285 | 0.96 |
| ZFP69B | 146 | 41 | 0.28 | VSX1 | 561 | 1516 | 2.70 | CCDC74A | 438 | 414 | 0.95 |
| IBTK | 364 | 102 | 0.28 | ERP29 | 219 | 590 | 2.69 | DUSP8 | 88 | 83 | 0.94 |
| CXCR1 | 204 | 56 | 0.27 | OMD | 322 | 865 | 2.69 | PPP2R5E | 193 | 181 | 0.94 |
| hsa-mir-1182 | 51 | 14 | 0.27 | AICDA | 151 | 404 | 2.68 | ORC5 | 484 | 445 | 0.92 |
| TMEM213 | 128 | 35 | 0.27 | ANTXR1 | 18 | 48 | 2.67 | CCDC88C | 147 | 135 | 0.92 |
| PRSS8 | 549 | 150 | 0.27 | KPNA2 | 112 | 298 | 2.66 | CD19 | 356 | 321 | 0.90 |
| MAP2K7 | 112 | 30 | 0.27 | PKP4 | 78 | 205 | 2.63 | FAM175B | 182 | 164 | 0.90 |
| hsa-mir-492 | 273 | 73 | 0.27 | TUFM | 247 | 643 | 2.60 | ZSWIM2 | 421 | 375 | 0.89 |
| FLVCR2 | 185 | 49 | 0.26 | GJA3 | 303 | 788 | 2.60 | GLIPR2 | 166 | 147 | 0.89 |
| FSCN2 | 68 | 18 | 0.26 | FBX018 | 235 | 611 | 2.60 | ERAL1 | 224 | 198 | 0.88 |
| HCK | 87 | 23 | 0.26 | TMIGD2 | 88 | 228 | 2.59 | CLCN7 | 445 | 390 | 0.88 |
| CLLU1OS | 250 | 66 | 0.26 | USP16 | 361 | 935 | 2.59 | COL4A6 | 102 | 89 | 0.87 |
| BCKDHA | 163 | 43 | 0.26 | HP1BP3 | 169 | 435 | 2.57 | PRPH2 | 112 | 97 | 0.87 |
| ZNF770 | 133 | 35 | 0.26 | SRMS | 168 | 431 | 2.57 | CTSV | 261 | 226 | 0.87 |
| POLR1E | 76 | 20 | 0.26 | C17orf112 | 132 | 337 | 2.55 | PCNX | 94 | 81 | 0.86 |
| SLC8A1 | 88 | 23 | 0.26 | TSFM | 11 | 28 | 2.55 | FOPNL | 114 | 98 | 0.86 |
| DCAF6 | 174 | 45 | 0.26 | NQO1 | 288 | 733 | 2.55 | ZNF581 | 139 | 119 | 0.86 |
| KIAA1024L | 396 | 102 | 0.26 | TRAPPC5 | 162 | 412 | 2.54 | CD3G | 468 | 399 | 0.85 |
| hsa-mir-6778 | 231 | 59 | 0.26 | DENR | 29 | 73 | 2.52 | EMC6 | 161 | 137 | 0.85 |
| FGL2 | 149 | 38 | 0.26 | hsa-mir-1277 | 308 | 774 | 2.51 | INADL | 92 | 78 | 0.85 |
| EBP | 564 | 142 | 0.25 | CDCA7L | 222 | 553 | 2.49 | TIE1 | 332 | 281 | 0.85 |
| FAM153A | 864 | 217 | 0.25 | SLC2A14 | 110 | 273 | 2.48 | CCDC134 | 457 | 385 | 0.84 |
| METAP1D | 52 | 13 | 0.25 | hsa-mir-645 | 102 | 253 | 2.48 | NAA35 | 139 | 117 | 0.84 |
| PRB3 | 279 | 69 | 0.25 | TSHR | 196 | 486 | 2.48 | RWDD2B | 131 | 110 | 0.84 |
| UBAC2 | 267 | 65 | 0.24 | EMC6 | 175 | 433 | 2.47 | RNF215 | 710 | 596 | 0.84 |
| PCDHA9 | 153 | 37 | 0.24 | COMTD1 | 434 | 1063 | 2.45 | ZIC2 | 143 | 120 | 0.84 |
| ADRM1 | 397 | 96 | 0.24 | RAP1GAP | 273 | 667 | 2.44 | ZNF79 | 1043 | 864 | 0.83 |
| SVOPL | 406 | 98 | 0.24 | CD300LF | 199 | 482 | 2.42 | SNX16 | 290 | 239 | 0.82 |
| MTERFD3 | 531 | 128 | 0.24 | MPPED1 | 186 | 447 | 2.40 | MSX2 | 78 | 64 | 0.82 |
| MAP9 | 241 | 57 | 0.24 | FAM47B | 40 | 96 | 2.40 | GPBP1 | 122 | 99 | 0.81 |
| FMR1NB | 102 | 24 | 0.24 | SMARCA2 | 147 | 352 | 2.39 | MLXIP | 163 | 132 | 0.81 |
| ABHD14B | 64 | 15 | 0.23 | ADH5 | 190 | 454 | 2.39 | MINOS1 | 227 | 183 | 0.81 |
| TAS2R19 | 444 | 104 | 0.23 | SLC10A7 | 522 | 1238 | 2.37 | CCDC13 | 313 | 251 | 0.80 |
| PPA1 | 284 | 66 | 0.23 | ZFP91 | 127 | 298 | 2.35 | SRPK3 | 349 | 279 | 0.80 |
| BRD2 | 194 | 45 | 0.23 | LOC100506388 | 441 | 1033 | 2.34 | SST | 357 | 285 | 0.80 |
| ENTPD7 | 441 | 101 | 0.23 | KIF15 | 199 | 466 | 2.34 | ZNF786 | 550 | 435 | 0.79 |
| hsa-mir-2116 | 206 | 47 | 0.23 | FBX048 | 538 | 1256 | 2.33 | EEF1B2 | 42 | 33 | 0.79 |
| UBXN11 | 197 | 44 | 0.22 | PTGIS | 160 | 373 | 2.33 | MMP21 | 606 | 472 | 0.78 |
| hsa-mir-758 | 701 | 156 | 0.22 | PSG5 | 381 | 886 | 2.33 | ARHGEF5 | 163 | 126 | 0.77 |
| hsa-mir-1289-1 | 207 | 46 | 0.22 | FN3KRP | 245 | 565 | 2.31 | CD33 | 163 | 126 | 0.77 |
| RHEBL1 | 9 | 2 | 0.22 | SPINT2 | 107 | 244 | 2.28 | ZNF491 | 245 | 189 | 0.77 |
| OR5H2 | 221 | 49 | 0.22 | MTUS2 | 194 | 442 | 2.28 | C2orf83 | 165 | 127 | 0.77 |
| HLA-F | 193 | 42 | 0.22 | ADAT1 | 27 | 61 | 2.26 | YIPF7 | 502 | 380 | 0.76 |
| PDIA6 | 92 | 20 | 0.22 | WWC2 | 273 | 614 | 2.25 | HTR2B | 415 | 314 | 0.76 |
| LZTFL1 | 69 | 15 | 0.22 | hsa-mir-23c | 145 | 326 | 2.25 | GTF3C5 | 273 | 206 | 0.75 |
| FHL3 | 65 | 14 | 0.22 | UEVLD | 291 | 654 | 2.25 | ATP8A2 | 524 | 395 | 0.75 |
| hsa-mir-200c | 451 | 97 | 0.22 | hsa-mir-5697 | 251 | 563 | 2.24 | CCL14 | 539 | 403 | 0.75 |
| DAAM2 | 280 | 60 | 0.21 | ABCB6 | 186 | 416 | 2.24 | CCDC108 | 414 | 309 | 0.75 |
| ZC3HAV1 | 196 | 42 | 0.21 | PYDC1 | 192 | 428 | 2.23 | GDPD5 | 332 | 247 | 0.74 |
| EXT2 | 99 | 21 | 0.21 | AKT2 | 89 | 198 | 2.22 | TEX15 | 233 | 173 | 0.74 |
| CLRN3 | 302 | 64 | 0.21 | WIPF1 | 499 | 1106 | 2.22 | TENC1 | 186 | 138 | 0.74 |
| ABCC2 | 90 | 19 | 0.21 | THUMPD1 | 275 | 609 | 2.21 | CNOT7 | 364 | 270 | 0.74 |
| SSTR3 | 133 | 28 | 0.21 | GSN | 441 | 972 | 2.20 | SNAPC5 | 173 | 128 | 0.74 |
| DPP9 | 281 | 59 | 0.21 | C8A | 412 | 907 | 2.20 | C5orf49 | 259 | 191 | 0.74 |
| ALKBH 7 | 447 | 93 | 0.21 | CEACAM7 | 138 | 302 | 2.19 | ITGB1BP2 | 387 | 285 | 0.74 |
| OR52L1 | 267 | 55 | 0.21 | EPHA1 | 70 | 153 | 2.19 | B4GALT2 | 269 | 198 | 0.74 |
| LRIT1 | 122 | 25 | 0.20 | OR52E2 | 18 | 39 | 2.17 | ACOT13 | 303 | 223 | 0.74 |
| MXI1 | 124 | 25 | 0.20 | TEX101 | 184 | 398 | 2.16 | HABP2 | 331 | 242 | 0.73 |
| C11orf82 | 273 | 55 | 0.20 | RAD23B | 186 | 402 | 2.16 | ACSM2B | 556 | 405 | 0.73 |
| hsa-mir-4421 | 5 | 1 | 0.20 | SSR3 | 252 | 535 | 2.12 | CNTN4 | 327 | 237 | 0.72 |
| EPC1 | 431 | 86 | 0.20 | CCDC144NL | 225 | 475 | 2.11 | CELSR3 | 112 | 81 | 0.72 |
| NDRG4 | 393 | 78 | 0.20 | hsa-mir-4779 | 169 | 355 | 2.10 | AP5M1 | 341 | 246 | 0.72 |
| KLHL7 | 162 | 32 | 0.20 | VARS2 | 88 | 184 | 2.09 | SH3TC2 | 185 | 133 | 0.72 |
| METAP1 | 291 | 57 | 0.20 | CBX7 | 254 | 529 | 2.08 | AIM1 | 213 | 153 | 0.72 |
| ATOH7 | 97 | 19 | 0.20 | ATP6AP1 | 466 | 967 | 2.08 | INSR | 340 | 244 | 0.72 |
| HMP19 | 128 | 25 | 0.20 | LSMEM1 | 151 | 313 | 2.07 | CXADR | 194 | 139 | 0.72 |
| C6orf70 | 134 | 26 | 0.19 | FCHO2 | 48 | 99 | 2.06 | EAPP | 305 | 218 | 0.71 |
| PCDHB14 | 182 | 35 | 0.19 | PDYN | 143 | 292 | 2.04 | SLC35B2 | 313 | 223 | 0.71 |
| ZNF695 | 236 | 45 | 0.19 | HEATR3 | 77 | 157 | 2.04 | C5orf15 | 152 | 108 | 0.71 |
| FAM114A2 | 273 | 52 | 0.19 | THRA | 26 | 53 | 2.04 | KDM4D | 114 | 81 | 0.71 |
| FGG | 428 | 81 | 0.19 | ENPP3 | 149 | 303 | 2.03 | ZNF835 | 270 | 191 | 0.71 |
| RPP40 | 106 | 20 | 0.19 | STC2 | 184 | 374 | 2.03 | ZNF408 | 149 | 105 | 0.70 |
| PYCARD | 203 | 38 | 0.19 | ERLEC1 | 221 | 445 | 2.01 | BTBD10 | 267 | 188 | 0.70 |
| ZBTB42 | 391 | 73 | 0.19 | CDS1 | 327 | 657 | 2.01 | PAPOLA | 27 | 19 | 0.70 |
| hsa-mir-4276 | 162 | 30 | 0.19 | SYVN1 | 238 | 478 | 2.01 | ZNF586 | 157 | 110 | 0.70 |
| CCL4 | 348 | 64 | 0.18 | hsa-mir-6824 | 463 | 929 | 2.01 | SPATA4 | 353 | 247 | 0.70 |
| NUDT2 | 490 | 89 | 0.18 | PRR22 | 67 | 132 | 1.97 | DDOST | 457 | 318 | 0.70 |
| FAM21B | 281 | 51 | 0.18 | ACSL6 | 132 | 260 | 1.97 | TMEM159 | 495 | 343 | 0.69 |
| OR6N1 | 238 | 43 | 0.18 | TMEM100 | 63 | 124 | 1.97 | PRMT5 | 314 | 217 | 0.69 |
| SH3YL1 | 195 | 35 | 0.18 | ANAPC2 | 154 | 303 | 1.97 | OR4C13 | 157 | 107 | 0.68 |
| LOC100288524 | 301 | 54 | 0.18 | ALOX12B | 241 | 471 | 1.95 | P4HA3 | 781 | 532 | 0.68 |
| FLT3 | 552 | 99 | 0.18 | hsa-mir-20b | 509 | 993 | 1.95 | NLGN3 | 456 | 308 | 0.68 |
| CFLAR | 147 | 26 | 0.18 | NEIL2 | 411 | 793 | 1.93 | C9orf91 | 351 | 237 | 0.68 |
| PTPRC | 221 | 39 | 0.18 | OR6K2 | 228 | 439 | 1.93 | C15orf62 | 273 | 182 | 0.67 |
| TPTE2 | 17 | 3 | 0.18 | OR5L1 | 277 | 533 | 1.92 | TM4SF18 | 244 | 161 | 0.66 |
| DAPL1 | 159 | 28 | 0.18 | PTPRB | 118 | 227 | 1.92 | C1orf100 | 75 | 49 | 0.65 |
| HYLS1 | 239 | 42 | 0.18 | hsa-mir-4674 | 150 | 288 | 1.92 | KIF20A | 679 | 440 | 0.65 |
| SPACA4 | 97 | 17 | 0.18 | NR2C2AP | 37 | 71 | 1.92 | PELI1 | 252 | 162 | 0.64 |
| GPATCH2 | 556 | 97 | 0.17 | CRYM | 17 | 32 | 1.88 | CNST | 223 | 143 | 0.64 |
| ZNF77 | 310 | 54 | 0.17 | CD40LG | 168 | 316 | 1.88 | ABLIM1 | 508 | 325 | 0.64 |
| VAMP2 | 219 | 38 | 0.17 | SLC2A3 | 72 | 135 | 1.88 | LY86 | 157 | 100 | 0.64 |
| C1orf174 | 173 | 30 | 0.17 | STX19 | 253 | 473 | 1.87 | DKK1 | 138 | 87 | 0.63 |
| TSSK6 | 243 | 42 | 0.17 | CYP19A1 | 183 | 342 | 1.87 | SLTM | 73 | 46 | 0.63 |
| ANKRD28 | 369 | 63 | 0.17 | PSG6 | 325 | 607 | 1.87 | IL11 | 154 | 97 | 0.63 |
| CLRN2 | 223 | 38 | 0.17 | PLIN5 | 124 | 231 | 1.86 | GBE1 | 54 | 34 | 0.63 |
| ZC2HC1A | 94 | 16 | 0.17 | GPR161 | 113 | 208 | 1.84 | IFIT3 | 310 | 195 | 0.63 |
| TGFBR1 | 316 | 53 | 0.17 | EXT1 | 219 | 403 | 1.84 | DIAPH3 | 197 | 123 | 0.62 |
| STK31 | 373 | 62 | 0.17 | ANO1 | 236 | 434 | 1.84 | NAA15 | 527 | 328 | 0.62 |
| CD163 | 121 | 20 | 0.17 | hsa-mir-377 | 86 | 158 | 1.84 | SNRNP25 | 305 | 189 | 0.62 |
| SAGE1 | 294 | 48 | 0.16 | AFP | 226 | 415 | 1.84 | PGLYRP3 | 206 | 126 | 0.61 |
| ITFG1 | 184 | 30 | 0.16 | CTDSP1 | 218 | 396 | 1.82 | INTS12 | 347 | 211 | 0.61 |
| SLC37A2 | 351 | 57 | 0.16 | AQP10 | 291 | 528 | 1.81 | RFK | 160 | 97 | 0.61 |
| GCSAM | 111 | 18 | 0.16 | NKX1-2 | 75 | 136 | 1.81 | EXOC3 | 88 | 52 | 0.59 |
| CACNG1 | 205 | 33 | 0.16 | MVD | 118 | 213 | 1.81 | CERKL | 275 | 162 | 0.59 |
| AZI2 | 201 | 32 | 0.16 | TMPRSS11D | 71 | 128 | 1.80 | ABHD12 | 461 | 271 | 0.59 |
| CYP1A2 | 45 | 7 | 0.16 | C16orf72 | 227 | 409 | 1.80 | SLC22A6 | 223 | 131 | 0.59 |
| IGDCC4 | 52 | 8 | 0.15 | KIAA0907 | 424 | 759 | 1.79 | GNG10 | 175 | 102 | 0.58 |
| AMER3 | 13 | 2 | 0.15 | HDAC5 | 376 | 673 | 1.79 | TMED7- | 534 | 311 | 0.58 |
| | | | | | | | | TICAM2 | | | |
| FAM110D | 438 | 67 | 0.15 | VIP | 78 | 139 | 1.78 | RNASE4 | 358 | 207 | 0.58 |
| DTNB | 113 | 17 | 0.15 | KCNB2 | 150 | 267 | 1.78 | NRAP | 410 | 236 | 0.58 |
| hsa-mir-513c | 88 | 13 | 0.15 | MLXIP | 45 | 80 | 1.78 | C4BPB | 42 | 24 | 0.57 |
| RAD51 | 285 | 42 | 0.15 | APOBEC3F | 210 | 373 | 1.78 | RAB26 | 302 | 172 | 0.57 |
| P2RY1 | 163 | 24 | 0.15 | PYGM | 308 | 547 | 1.78 | HIST1H2AG | 390 | 222 | 0.57 |
| ANKRD20A1 | 34 | 5 | 0.15 | ARRDC2 | 113 | 200 | 1.77 | GTF2H2D | 60 | 34 | 0.57 |
| ZCCHC14 | 48 | 7 | 0.15 | hsa-mir-4769 | 56 | 99 | 1.77 | IPP | 825 | 466 | 0.56 |
| hsa-mir-551a | 174 | 25 | 0.14 | KCNC4 | 302 | 527 | 1.75 | RARRES3 | 514 | 290 | 0.56 |
| hsa-mir-4678 | 42 | 6 | 0.14 | HNRNPH1 | 298 | 519 | 1.74 | SRR | 695 | 391 | 0.56 |
| C10orf90 | 220 | 31 | 0.14 | KIF1B | 89 | 154 | 1.73 | SDR42E1 | 136 | 75 | 0.55 |
| PCDHB10 | 57 | 8 | 0.14 | LRP2 | 321 | 555 | 1.73 | KRT3 | 565 | 311 | 0.55 |
| MYL12B | 646 | 90 | 0.14 | VN1R5 | 253 | 437 | 1.73 | ZNF555 | 255 | 140 | 0.55 |
| CLEC17A | 133 | 18 | 0.14 | SLPI | 220 | 380 | 1.73 | BAIAP2 | 596 | 327 | 0.55 |
| KLHL3 | 215 | 29 | 0.13 | FAM71C | 143 | 247 | 1.73 | TANC1 | 483 | 265 | 0.55 |
| PPP5C | 157 | 21 | 0.13 | OR4Q3 | 43 | 74 | 1.72 | GFOD2 | 174 | 95 | 0.55 |
| RAPSN | 15 | 2 | 0.13 | SOWAHC | 235 | 404 | 1.72 | ZNF101 | 194 | 105 | 0.54 |
| PINX1 | 219 | 29 | 0.13 | SPINK14 | 7 | 12 | 1.71 | ENG | 320 | 173 | 0.54 |
| ESRP2 | 144 | 19 | 0.13 | BIRC5 | 114 | 195 | 1.71 | OR5H2 | 288 | 155 | 0.54 |
| CYB5A | 147 | 19 | 0.13 | RABL3 | 230 | 389 | 1.69 | WISP1 | 164 | 88 | 0.54 |
| VCX2 | 217 | 28 | 0.13 | CCR6 | 314 | 527 | 1.68 | LYPD1 | 235 | 126 | 0.54 |
| OST4 | 527 | 67 | 0.13 | OSCAR | 93 | 155 | 1.67 | NFKB1 | 162 | 86 | 0.53 |
| SGPP1 | 269 | 34 | 0.13 | CLCN5 | 83 | 138 | 1.66 | PLCL2 | 276 | 146 | 0.53 |
| KLHL2 | 319 | 40 | 0.13 | KIF13A | 126 | 209 | 1.66 | BHLHE41 | 82 | 43 | 0.52 |
| PLA2G10 | 639 | 80 | 0.13 | MX1 | 315 | 518 | 1.64 | TNFSF4 | 284 | 148 | 0.52 |
| SERP1 | 104 | 13 | 0.13 | NR1H4 | 213 | 350 | 1.64 | DPCR1 | 142 | 74 | 0.52 |
| DENR | 8 | 1 | 0.13 | TRPC6 | 230 | 373 | 1.62 | CA12 | 224 | 116 | 0.52 |
| PDIK1L | 211 | 26 | 0.12 | ZNF57 | 359 | 577 | 1.61 | RERE | 141 | 73 | 0.52 |
| PEBP1 | 180 | 22 | 0.12 | TREM1 | 217 | 347 | 1.60 | BCAN | 114 | 59 | 0.52 |
| MED7 | 304 | 37 | 0.12 | RGP1 | 402 | 640 | 1.59 | KRT12 | 177 | 91 | 0.51 |
| HTN3 | 189 | 23 | 0.12 | FAM115C | 277 | 439 | 1.58 | BCL2L13 | 283 | 143 | 0.51 |
| UBE2Q1 | 222 | 27 | 0.12 | COX7A2 | 110 | 174 | 1.58 | CTCFL | 321 | 162 | 0.50 |
| KRTAP10-4 | 141 | 17 | 0.12 | CAPRIN1 | 597 | 938 | 1.57 | LYPD4 | 298 | 149 | 0.50 |
| XCR1 | 83 | 10 | 0.12 | EIF5B | 72 | 113 | 1.57 | DOHH | 54 | 27 | 0.50 |
| ARPP21 | 167 | 20 | 0.12 | ZNF488 | 220 | 342 | 1.55 | CCDC166 | 22 | 11 | 0.50 |
| NAALAD2 | 59 | 7 | 0.12 | ZNF318 | 150 | 233 | 1.55 | MDH2 | 247 | 123 | 0.50 |
| TIRAP | 287 | 34 | 0.12 | CCDC24 | 96 | 149 | 1.55 | RTTN | 181 | 90 | 0.50 |
| ZNF883 | 423 | 50 | 0.12 | ARHGAP22 | 127 | 197 | 1.55 | CHCHD7 | 212 | 105 | 0.50 |
| CMTM2 | 128 | 15 | 0.12 | hsa-mir-299 | 274 | 424 | 1.55 | HMX2 | 85 | 42 | 0.49 |
| EHF | 293 | 34 | 0.12 | PLA2G10 | 689 | 1066 | 1.55 | UBE2D4 | 334 | 165 | 0.49 |
| KLHL9 | 181 | 21 | 0.12 | hsa-mir-196b | 151 | 232 | 1.54 | KLRG1 | 189 | 93 | 0.49 |
| TRAPPC8 | 315 | 36 | 0.11 | CXCR4 | 156 | 239 | 1.53 | ANAPC7 | 316 | 155 | 0.49 |
| SEMA6A | 168 | 19 | 0.11 | hsa-mir-5687 | 115 | 176 | 1.53 | SF3B14 | 351 | 171 | 0.49 |
| VWA5B2 | 142 | 16 | 0.11 | LARGE | 168 | 257 | 1.53 | KSR2 | 187 | 91 | 0.49 |
| LOC440563 | 45 | 5 | 0.11 | ARIH2 | 294 | 449 | 1.53 | NIPSNAP3B | 101 | 49 | 0.49 |
| hsa-mir-154 | 9 | 1 | 0.11 | B4GALT2 | 105 | 160 | 1.52 | ABCF2 | 385 | 186 | 0.48 |
| TERT | 226 | 25 | 0.11 | NEURL4 | 156 | 235 | 1.51 | UXS1 | 118 | 57 | 0.48 |
| CLEC4D | 182 | 20 | 0.11 | OR6V1 | 342 | 515 | 1.51 | MAGEA12 | 292 | 141 | 0.48 |
| hsa-mir-628 | 110 | 12 | 0.11 | hsa-mir-4654 | 186 | 280 | 1.51 | SLC4A1AP | 460 | 221 | 0.48 |
| PKHD1LI | 230 | 25 | 0.11 | APOL2 | 93 | 140 | 1.51 | SH3YL1 | 465 | 223 | 0.48 |
| hsa-mir-217 | 92 | 10 | 0.11 | TMSB10 | 194 | 292 | 1.51 | PLA2G4B | 484 | 232 | 0.48 |
| ADAM17 | 378 | 41 | 0.11 | NRL | 196 | 294 | 1.50 | SASH3 | 84 | 40 | 0.48 |
| TCTEX1D4 | 93 | 10 | 0.11 | TLR2 | 269 | 403 | 1.50 | CLN8 | 259 | 123 | 0.47 |
| TWISTNB | 298 | 32 | 0.11 | RASA1 | 121 | 181 | 1.50 | ACTN4 | 57 | 27 | 0.47 |
| UBAP2L | 149 | 16 | 0.11 | IFNA10 | 610 | 912 | 1.50 | TMEM191C | 146 | 69 | 0.47 |
| OR10G4 | 112 | 12 | 0.11 | TTC7A | 116 | 173 | 1.49 | IMPAD1 | 345 | 163 | 0.47 |
| hsa-mir-4494 | 131 | 14 | 0.11 | FOXD3 | 72 | 107 | 1.49 | GALNT13 | 575 | 269 | 0.47 |
| UBE2V1 | 113 | 12 | 0.11 | C1QL1 | 373 | 552 | 1.48 | ELL2 | 620 | 289 | 0.47 |
| hsa-mir-5584 | 396 | 42 | 0.11 | ITGA10 | 174 | 256 | 1.47 | LRRC8E | 251 | 116 | 0.46 |
| FBXL20 | 265 | 28 | 0.11 | SIGLEC7 | 62 | 91 | 1.47 | TBC1D29 | 362 | 166 | 0.46 |
| hsa-mir-383 | 771 | 81 | 0.11 | SLC3A1 | 340 | 499 | 1.47 | CHRNA4 | 132 | 60 | 0.45 |
| CLCC1 | 182 | 19 | 0.10 | hsa-mir-8068 | 75 | 110 | 1.47 | ALDH1L1 | 22 | 10 | 0.45 |
| GTF2A1 | 154 | 16 | 0.10 | NEUROD4 | 176 | 257 | 1.46 | MIP | 214 | 97 | 0.45 |
| DNAJC3 | 799 | 81 | 0.10 | ZNF346 | 59 | 86 | 1.46 | FAM162A | 759 | 343 | 0.45 |
| MMP28 | 168 | 17 | 0.10 | TMEM186 | 326 | 475 | 1.46 | KRTCAP2 | 175 | 79 | 0.45 |
| PSMD13 | 277 | 28 | 0.10 | CXCR5 | 125 | 182 | 1.46 | NTAN1 | 100 | 45 | 0.45 |
| CBFB | 357 | 36 | 0.10 | STOML2 | 121 | 176 | 1.45 | CYYR1 | 147 | 66 | 0.45 |
| AP4E1 | 130 | 13 | 0.10 | hsa-mir-6797 | 33 | 48 | 1.45 | EMC6 | 171 | 76 | 0.44 |
| CALB2 | 10 | 1 | 0.10 | CPEB2 | 565 | 821 | 1.45 | STOM | 363 | 161 | 0.44 |
| METTL17 | 171 | 17 | 0.10 | TOP2B | 274 | 398 | 1.45 | BCL2L11 | 428 | 189 | 0.44 |
| TPPP | 550 | 54 | 0.10 | REEP4 | 125 | 181 | 1.45 | BDNF | 222 | 98 | 0.44 |
| LOC154872 | 369 | 36 | 0.10 | GYLTL1B | 102 | 146 | 1.43 | LRRC14 | 114 | 50 | 0.44 |
| SEMA6C | 72 | 7 | 0.10 | HMP19 | 91 | 130 | 1.43 | IL12B | 57 | 25 | 0.44 |
| NAP1L5 | 319 | 31 | 0.10 | ABR | 165 | 235 | 1.42 | MYB | 504 | 221 | 0.44 |
| DNAJC2 | 228 | 22 | 0.10 | L1CAM | 148 | 210 | 1.42 | PATE1 | 211 | 92 | 0.44 |
| RB1 | 685 | 66 | 0.10 | ZNF286B | 78 | 110 | 1.41 | CDH20 | 186 | 80 | 0.43 |
| CISD1 | 461 | 44 | 0.10 | RBM6 | 98 | 138 | 1.41 | NWD1 | 119 | 51 | 0.43 |
| SRPK2 | 42 | 4 | 0.10 | CROCC | 56 | 78 | 1.39 | PDXP | 7 | 3 | 0.43 |
| HNRNPF | 21 | 2 | 0.10 | AGPAT6 | 90 | 125 | 1.39 | NLRP1 | 150 | 64 | 0.43 |
| KIAA1430 | 200 | 19 | 0.10 | KRTAP27-1 | 50 | 69 | 1.38 | PHLDA3 | 214 | 90 | 0.42 |
| MANEAL | 443 | 42 | 0.09 | PRLHR | 241 | 332 | 1.38 | OR5D16 | 239 | 100 | 0.42 |
| ARHGEF1 | 285 | 27 | 0.09 | hsa-mir-5089 | 134 | 184 | 1.37 | SYNDIG1L | 787 | 326 | 0.41 |
| hsa-mir-183 | 454 | 43 | 0.09 | FAM129C | 207 | 284 | 1.37 | DHRS7C | 237 | 98 | 0.41 |
| RASGEF1B | 286 | 27 | 0.09 | COPS7B | 297 | 405 | 1.36 | CES1 | 199 | 82 | 0.41 |
| ZMYND10 | 435 | 41 | 0.09 | SPCS2 | 22 | 30 | 1.36 | NDRG2 | 261 | 107 | 0.41 |
| LHX5 | 340 | 32 | 0.09 | SPATA19 | 127 | 173 | 1.36 | BRS3 | 244 | 100 | 0.41 |
| PTPRE | 812 | 76 | 0.09 | ATRN | 377 | 512 | 1.36 | TCF19 | 159 | 65 | 0.41 |
| CHMP4A | 294 | 27 | 0.09 | AQPEP | 263 | 356 | 1.35 | IQCB1 | 306 | 125 | 0.41 |
| MIS18BP1 | 98 | 9 | 0.09 | EEF1G | 51 | 69 | 1.35 | SETD1A | 642 | 262 | 0.41 |
| RASSF8 | 98 | 9 | 0.09 | TOPAZ1 | 179 | 240 | 1.34 | DDX27 | 302 | 123 | 0.41 |
| SOX13 | 297 | 27 | 0.09 | AXIN1 | 77 | 103 | 1.34 | RBM44 | 106 | 43 | 0.41 |
| ABCC9 | 11 | 1 | 0.09 | TMEM199 | 113 | 151 | 1.34 | C3orf72 | 227 | 92 | 0.41 |
| ARHGEF12 | 11 | 1 | 0.09 | TAZ | 143 | 191 | 1.34 | CDC25C | 385 | 156 | 0.41 |
| C12orf60 | 264 | 23 | 0.09 | BEST4 | 332 | 442 | 1.33 | NCR1 | 358 | 145 | 0.41 |
| TMPRSS6 | 138 | 12 | 0.09 | GPR112 | 160 | 213 | 1.33 | MAP1LC3B2 | 159 | 64 | 0.40 |
| RNF185 | 461 | 40 | 0.09 | BROX | 483 | 641 | 1.33 | MYO1G | 110 | 44 | 0.40 |
| ORC2 | 58 | 5 | 0.09 | MAPKBP1 | 178 | 236 | 1.33 | FRS3 | 78 | 31 | 0.40 |
| SEMA3C | 199 | 17 | 0.09 | YIPF4 | 203 | 269 | 1.33 | IGJ | 172 | 68 | 0.40 |
| LPPR5 | 285 | 24 | 0.08 | hsa-mir-7703 | 31 | 41 | 1.32 | NFKBID | 363 | 143 | 0.39 |
| MNT | 264 | 22 | 0.08 | FAM110C | 47 | 62 | 1.32 | NANOG | 216 | 85 | 0.39 |
| ARSJ | 48 | 4 | 0.08 | SPHAR | 29 | 38 | 1.31 | DNASE2B | 102 | 40 | 0.39 |
| OR56B4 | 327 | 27 | 0.08 | BRD4 | 614 | 804 | 1.31 | ZFYVE26 | 82 | 32 | 0.39 |
| CEP250 | 73 | 6 | 0.08 | C1orf194 | 145 | 189 | 1.30 | SYCE3 | 510 | 199 | 0.39 |
| NYX | 225 | 18 | 0.08 | VWA5B2 | 290 | 377 | 1.30 | ZNF664- | 217 | 84 | 0.39 |
| | | | | | | | | FAM101A | | | |
| hsa-mir-132 | 100 | 8 | 0.08 | CERKL | 246 | 319 | 1.30 | CEBPG | 223 | 86 | 0.39 |
| R3HCC1L | 113 | 9 | 0.08 | SOST | 54 | 70 | 1.30 | KDM4E | 236 | 91 | 0.39 |
| NT5C1B | 165 | 13 | 0.08 | SPG20 | 350 | 453 | 1.29 | AVPR2 | 205 | 79 | 0.39 |
| CCDC160 | 128 | 10 | 0.08 | ULBP3 | 131 | 169 | 1.29 | NSMAF | 330 | 127 | 0.38 |
| ERLIN2 | 90 | 7 | 0.08 | MAGEB10 | 45 | 57 | 1.27 | SPINK6 | 169 | 65 | 0.38 |
| ACAN | 309 | 24 | 0.08 | GAB1 | 166 | 210 | 1.27 | ZNF22 | 241 | 92 | 0.38 |
| BICD2 | 362 | 28 | 0.08 | NEK5 | 267 | 337 | 1.26 | CLEC7A | 160 | 61 | 0.38 |
| ASB1 | 569 | 44 | 0.08 | C2orf44 | 313 | 395 | 1.26 | PTPRO | 193 | 73 | 0.38 |
| FAM3D | 273 | 21 | 0.08 | HMGA2 | 463 | 583 | 1.26 | CES5A | 766 | 289 | 0.38 |
| NFKBIL1 | 26 | 2 | 0.08 | PTTG2 | 255 | 321 | 1.26 | FHOD1 | 345 | 130 | 0.38 |
| SPINK14 | 13 | 1 | 0.08 | ALCAM | 209 | 263 | 1.26 | ATF3 | 316 | 119 | 0.38 |
| MBD5 | 378 | 29 | 0.08 | RHOBTB3 | 194 | 244 | 1.26 | SPTBN4 | 170 | 64 | 0.38 |
| DNAJC16 | 170 | 13 | 0.08 | ACSF2 | 156 | 195 | 1.25 | SPCS1 | 75 | 28 | 0.37 |
| TMEM123 | 158 | 12 | 0.08 | FAM47A | 20 | 25 | 1.25 | SPINK14 | 418 | 156 | 0.37 |
| GNAS | 79 | 6 | 0.08 | LPCAT2 | 90 | 112 | 1.24 | EGLN2 | 201 | 75 | 0.37 |
| DEFB118 | 358 | 27 | 0.08 | CEP44 | 64 | 79 | 1.23 | HILPDA | 183 | 68 | 0.37 |
| SGCZ | 347 | 26 | 0.07 | hsa-mir-376a-2 | 505 | 620 | 1.23 | CTLA4 | 253 | 94 | 0.37 |
| PCDP1 | 107 | 8 | 0.07 | TMEM114 | 314 | 385 | 1.23 | OR8B4 | 27 | 10 | 0.37 |
| C9orf64 | 228 | 17 | 0.07 | LRRC28 | 99 | 121 | 1.22 | FAM21C | 130 | 48 | 0.37 |
| hsa-mir-3671 | 121 | 9 | 0.07 | hsa-mir-1273g | 470 | 573 | 1.22 | ARMC2 | 271 | 100 | 0.37 |
| MKX | 175 | 13 | 0.07 | ASIP | 52 | 63 | 1.21 | ADCY6 | 125 | 46 | 0.37 |
| LILRB3 | 245 | 18 | 0.07 | OVCH2 | 53 | 64 | 1.21 | PLCG1 | 226 | 83 | 0.37 |
| INSM1 | 123 | 9 | 0.07 | LPIN3 | 58 | 70 | 1.21 | BTAF1 | 131 | 48 | 0.37 |
| CCDC106 | 411 | 30 | 0.07 | hsa-mir-4657 | 29 | 35 | 1.21 | HDAC4 | 202 | 74 | 0.37 |
| PGM1 | 274 | 20 | 0.07 | LSP1 | 103 | 124 | 1.20 | SOX15 | 706 | 258 | 0.37 |
| TRIML1 | 96 | 7 | 0.07 | CXCL5 | 208 | 250 | 1.20 | ZNF202 | 451 | 164 | 0.36 |
| RAB18 | 55 | 4 | 0.07 | RBP4 | 224 | 269 | 1.20 | PSMB11 | 942 | 342 | 0.36 |
| hsa-mir-29b-2 | 252 | 18 | 0.07 | MFGE8 | 85 | 102 | 1.20 | TCHHL1 | 711 | 257 | 0.36 |
| RAB37 | 84 | 6 | 0.07 | TAS2R30 | 36 | 43 | 1.19 | FZD7 | 183 | 66 | 0.36 |
| SLC37A1 | 70 | 5 | 0.07 | CSTF2T | 350 | 417 | 1.19 | GP5 | 379 | 136 | 0.36 |
| FAM19A1 | 323 | 23 | 0.07 | NABP1 | 144 | 169 | 1.17 | THNSL2 | 306 | 109 | 0.36 |
| SPAG7 | 127 | 9 | 0.07 | BST1 | 410 | 480 | 1.17 | PRG3 | 287 | 102 | 0.36 |
| STUB1 | 113 | 8 | 0.07 | RAB3IP | 102 | 119 | 1.17 | FCN3 | 197 | 70 | 0.36 |
| LST1 | 229 | 16 | 0.07 | GPBP1L1 | 139 | 161 | 1.16 | RAB1A | 364 | 129 | 0.35 |
| ATP5G1 | 276 | 19 | 0.07 | SON | 121 | 140 | 1.16 | ZGLP1 | 398 | 141 | 0.35 |
| MID2 | 204 | 14 | 0.07 | HN1L | 720 | 832 | 1.16 | HMGCLL1 | 211 | 74 | 0.35 |
| OR52E2 | 219 | 15 | 0.07 | EAF2 | 45 | 52 | 1.16 | CCDC67 | 341 | 119 | 0.35 |
| CPB1 | 73 | 5 | 0.07 | ADD2 | 291 | 336 | 1.15 | C1R | 109 | 38 | 0.35 |
| NUP188 | 176 | 12 | 0.07 | ST6GALNAC3 | 289 | 332 | 1.15 | DHRS4L1 | 609 | 212 | 0.35 |
| MAPK12 | 88 | 6 | 0.07 | KIF4A | 216 | 246 | 1.14 | KIAA1731 | 230 | 80 | 0.35 |
| ALDH16A1 | 44 | 3 | 0.07 | hsa-mir-4729 | 53 | 60 | 1.13 | PSMC6 | 72 | 25 | 0.35 |
| SPSB3 | 177 | 12 | 0.07 | CCM2L | 107 | 121 | 1.13 | PRM1 | 242 | 84 | 0.35 |
| ABL1 | 266 | 18 | 0.07 | LATS2 | 23 | 26 | 1.13 | PRKAA1 | 75 | 26 | 0.35 |
| ASB16 | 133 | 9 | 0.07 | IPCEF1 | 346 | 390 | 1.13 | TEX101 | 252 | 87 | 0.35 |
| CBX2 | 284 | 19 | 0.07 | CCDC132 | 342 | 385 | 1.13 | BOK | 271 | 93 | 0.34 |
| ODF3B | 180 | 12 | 0.07 | OR4K2 | 289 | 325 | 1.12 | LMO1 | 191 | 65 | 0.34 |
| B3GNT5 | 135 | 9 | 0.07 | SERPINI1 | 76 | 85 | 1.12 | SLC6A16 | 391 | 133 | 0.34 |
| CCKAR | 120 | 8 | 0.07 | NPY4R | 186 | 208 | 1.12 | WNT10A | 300 | 102 | 0.34 |
| PLCB2 | 75 | 5 | 0.07 | GNRH2 | 110 | 123 | 1.12 | CECR6 | 381 | 129 | 0.34 |
| LOC286238 | 166 | 11 | 0.07 | ZNF431 | 414 | 459 | 1.11 | GGCT | 101 | 34 | 0.34 |
| SPATA6 | 61 | 4 | 0.07 | SCYL3 | 74 | 82 | 1.11 | CD28 | 119 | 40 | 0.34 |
| SLFN14 | 168 | 11 | 0.07 | CAV3 | 205 | 227 | 1.11 | TBX3 | 499 | 167 | 0.33 |
| FAM175B | 550 | 36 | 0.07 | AGL | 357 | 394 | 1.10 | GPR83 | 273 | 91 | 0.33 |
| ZNF706 | 306 | 20 | 0.07 | hsa-mir-224 | 132 | 144 | 1.09 | INTU | 165 | 55 | 0.33 |
| WASF2 | 153 | 10 | 0.07 | SPTLC3 | 335 | 365 | 1.09 | MPZ | 99 | 33 | 0.33 |
| MAGEA8 | 123 | 8 | 0.07 | PHLDA3 | 123 | 134 | 1.09 | GRB7 | 239 | 79 | 0.33 |
| FABP6 | 400 | 26 | 0.07 | CCDC11 | 758 | 824 | 1.09 | C5orf51 | 106 | 35 | 0.33 |
| NCAPH2 | 31 | 2 | 0.06 | IL1F10 | 35 | 38 | 1.09 | TAS2R43 | 155 | 51 | 0.33 |
| hsa-mir-3684 | 280 | 18 | 0.06 | RAD51B | 95 | 103 | 1.08 | TMEM165 | 349 | 114 | 0.33 |
| MFSD1 | 140 | 9 | 0.06 | UNG | 107 | 116 | 1.08 | SERPINB4 | 98 | 32 | 0.33 |
| 0R4E2 | 327 | 21 | 0.06 | ARF4 | 337 | 365 | 1.08 | EDAR | 92 | 30 | 0.33 |
| HCN3 | 234 | 15 | 0.06 | NR2F2 | 53 | 57 | 1.08 | TGM5 | 210 | 68 | 0.32 |
| SETMAR | 236 | 15 | 0.06 | IFIT5 | 29 | 31 | 1.07 | SLC16A6 | 351 | 113 | 0.32 |
| FXYD5 | 571 | 36 | 0.06 | SEC61B | 239 | 255 | 1.07 | FOXB2 | 499 | 159 | 0.32 |
| ENAM | 143 | 9 | 0.06 | STK17B | 255 | 272 | 1.07 | RAC1 | 400 | 127 | 0.32 |
| ACTL6B | 32 | 2 | 0.06 | OR2M3 | 554 | 586 | 1.06 | CHRNA6 | 334 | 106 | 0.32 |
| hsa-mir-4328 | 403 | 25 | 0.06 | ZNF540 | 323 | 341 | 1.06 | EBLN2 | 462 | 146 | 0.32 |
| PLA2G5 | 178 | 11 | 0.06 | CCDC27 | 274 | 289 | 1.05 | DCXR | 285 | 90 | 0.32 |
| ZKSCAN4 | 260 | 16 | 0.06 | NKX3-1 | 148 | 156 | 1.05 | SLC30A9 | 57 | 18 | 0.32 |
| BPIFB6 | 65 | 4 | 0.06 | hsa-mir-376b | 97 | 102 | 1.05 | APOLD1 | 73 | 23 | 0.32 |
| HCK | 278 | 17 | 0.06 | MAP3K13 | 125 | 130 | 1.04 | ERI1 | 127 | 40 | 0.31 |
| SPINT2 | 131 | 8 | 0.06 | POU2F3 | 80 | 83 | 1.04 | RSBN1 | 143 | 45 | 0.31 |
| CHST11 | 279 | 17 | 0.06 | LCE6A | 192 | 196 | 1.02 | UNC13C | 387 | 121 | 0.31 |
| hsa-mir-4263 | 149 | 9 | 0.06 | hsa-mir-526a-1 | 48 | 49 | 1.02 | FKBP10 | 253 | 79 | 0.31 |
| OTOP1 | 414 | 25 | 0.06 | ZMYND8 | 280 | 285 | 1.02 | AMOT | 264 | 82 | 0.31 |
| ZFYVE21 | 829 | 50 | 0.06 | RAB3IL1 | 260 | 264 | 1.02 | OR2AT4 | 248 | 77 | 0.31 |
| FNDC1 | 166 | 10 | 0.06 | hsa-mir-30a | 294 | 297 | 1.01 | ABRA | 406 | 126 | 0.31 |
| UBE2G2 | 100 | 6 | 0.06 | CTNNA3 | 297 | 300 | 1.01 | VTA1 | 87 | 27 | 0.31 |
| NRXN2 | 50 | 3 | 0.06 | TAS2R13 | 148 | 149 | 1.01 | C13orf45 | 243 | 75 | 0.31 |
| RAD51AP1 | 367 | 22 | 0.06 | MAP1LC3C | 97 | 97 | 1.00 | MED6 | 26 | 8 | 0.31 |
| NQO2 | 419 | 25 | 0.06 | GDF6 | 47 | 47 | 1.00 | CASR | 186 | 57 | 0.31 |

TABLE 2
List of gene hits and scores.

| Screen 1—Technical repeat 1 | | | Screen 1—Technical repeat 2 | | | Screen 2 | | |
|---|---|---|---|---|---|---|---|---|
| gene | rank | effect_size | gene | rank | effect_size | gene | rank | effect_size |
| SEC63 | 1 | 6.65686 | SEC63 | 1 | 6.63909 | STT3A | 1 | 4.57001 |
| SPCS3 | 2 | 8.28261 | STT3A | 2 | 4.58143 | SEL1L | 2 | 7.67986 |
| SEC61B | 3 | 3.83774 | SEC61B | 3 | 4.19879 | EMC4 | 3 | 5.78404 |
| RAP1GAP | 4 | 9.75488 | OSTC | 4 | 4.57055 | SEC63 | 4 | 6.28183 |
| SLC35G5 | 5 | 8.25877 | SERP1 | 5 | 3.91901 | EMC3 | 5 | 3.45899 |
| OR6T1 | 6 | 7.74729 | SPCS3 | 6 | 10.6345 | MARK3 | 6 | 9.25255 |
| SEC61A1 | 7 | 7.69873 | ZYX | 7 | 8.21384 | CYP11B2 | 7 | 7.27094 |
| RRAS | 8 | 7.5599 | PARVG | 8 | 7.79001 | RPL23A | 8 | 7.24956 |
| RAB2B | 9 | 7.45755 | RAPSN | 9 | 7.13626 | KLRK1 | 9 | 7.15577 |
| SERP1 | 10 | 3.79056 | SLIT2 | 10 | 7.00096 | SERP1 | 10 | 6.79395 |
| SMAD4 | 11 | 6.70044 | CYB5A | 11 | 6.59212 | FAM212A | 11 | 6.7087 |
| CCSER2 | 12 | 6.72758 | TPTE2 | 12 | 6.50752 | CTSD | 12 | 6.60584 |
| OSTC | 13 | 6.68833 | FBXL20 | 13 | 6.3948 | SLC26A9 | 13 | 6.58576 |
| ATOH7 | 14 | 6.34179 | CYP1A2 | 14 | 6.36727 | ESPN | 14 | 6.54546 |
| LRRC37A3 | 15 | 6.2001 | AP1S3 | 15 | 6.21934 | PCP2 | 15 | 6.56191 |
| OST4 | 16 | 5.84208 | CPSF3L | 16 | 6.01095 | USB1 | 16 | 6.4 |
| FEZ2 | 17 | 5.71344 | ZKSCAN4 | 17 | 5.8358 | FIG4 | 17 | 6.35348 |
| TBPL1 | 18 | 5.57081 | hsa-mir-4421 | 18 | 5.78248 | TRERF1 | 18 | 6.31503 |
| DAPL1 | 19 | 5.56721 | HSPA13 | 19 | 5.64703 | PCDH9 | 19 | 6.27122 |
| CHCHD7 | 20 | 5.54647 | ALDH16A1 | 20 | 5.52185 | FBXL20 | 20 | 6.25805 |
| MMGT1 | 21 | 3.29686 | ASB16 | 21 | 5.48721 | GORAB | 21 | 6.1351 |
| PRH2 | 22 | 5.38824 | KCTD13 | 22 | 5.43248 | GKN2 | 22 | 6.1157 |
| CHMP1B | 23 | 5.37789 | PCDHGA6 | 23 | 5.45522 | UNKL | 23 | 6.07086 |
| SSR3 | 24 | 5.32014 | HIST1H2BI | 24 | 5.35364 | HSPA13 | 24 | 5.95852 |
| hsa-mir-6775 | 25 | 5.26677 | SCLT1 | 25 | 5.13587 | NCOA7 | 25 | 5.75767 |
| GGTLC1 | 26 | 5.08853 | ATG4D | 26 | 5.13277 | C12orf50 | 26 | 5.63995 |
| RBMX2 | 27 | 4.91974 | APBB3 | 27 | 4.99539 | DNAJC25 | 27 | 5.54295 |
| MORN2 | 28 | 4.84886 | ZSCAN23 | 28 | 4.88516 | KCP | 28 | 5.46617 |
| C11orf65 | 29 | 4.84833 | NCAPH2 | 29 | 4.90572 | ASPSCR1 | 29 | 5.49734 |
| TMEM232 | 30 | 4.57723 | FAM111A | 30 | 4.78874 | CD63 | 30 | 5.43052 |
| TAAR8 | 31 | 4.51472 | DDIT4 | 31 | 4.78743 | RGMB | 31 | 5.30957 |
| GZMM | 32 | 4.49035 | C1orf110 | 32 | 4.7761 | SSR3 | 32 | 5.09917 |
| PPP1R13B | 33 | 4.42226 | DNAJ B1 | 33 | 4.74875 | ADAMTS15 | 33 | 5.09282 |
| IL26 | 34 | 4.39677 | DCTN5 | 34 | 4.728 | PASK | 34 | 4.97399 |
| DNAJB14 | 35 | 4.28583 | CYP17A1 | 35 | 4.67326 | IFT20 | 35 | 4.88396 |
| NDST1 | 36 | 4.24682 | MED26 | 36 | 4.54364 | PNP | 36 | 4.80465 |
| hsa-mir-802 | 37 | 4.21247 | TBK1 | 37 | 4.5155 | PHF10 | 37 | 4.76692 |
| RER1 | 38 | 4.20327 | OR5L2 | 38 | 4.49653 | RAB39A | 38 | 4.68146 |
| CES4A | 39 | 4.18567 | AP4S1 | 39 | 4.48447 | TRIM21 | 39 | 4.66975 |
| COA5 | 40 | 4.10895 | ATP6AP1L | 40 | 4.41445 | DDR2 | 40 | 4.5992 |
| EMC6 | 41 | 2.45528 | FNBP4 | 41 | 4.41489 | GGT1 | 41 | 4.57118 |
| MOGS | 42 | 3.92186 | hsa-mir-4442 | 42 | 4.48664 | SYT10 | 42 | 4.56983 |
| PANX2 | 43 | 3.92521 | ABCC9 | 43 | 4.44742 | ANKIB1 | 43 | 4.48158 |
| RASA1 | 44 | 3.91723 | THRB | 44 | 4.39707 | BCAS1 | 44 | 4.41301 |
| EXO1 | 45 | 3.85964 | MKL2 | 45 | 4.34966 | ESF1 | 45 | 4.42205 |

TABLE 3
sgRNA sequence used for gene validation.

| | | SEQ |
|---|---|---|
| Gene | Spacer Sequence | ID NO: |
| AUP1 | AACCTGCGAAGGACGCTGTC | 1 |
| AUP1 | AGAGATTCTGTGCTTCCACG | 2 |
| AUP1 | CTGCTGCTCTACGCGCCAGT | 3 |
| B4GALT2 | TGCAGTCGGGCGGTGTGTAT | 4 |
| B4GALT2 | CCCTGTCCTGACTCGCCACC | 5 |
| B4GALT2 | GACCGCAACCTATACCGCTG | 6 |
| BRD3 | CGACGTGACGTTTGCAGTGA | 7 |
| BRD3 | ATTATTACCCCCTGCTCCAA | 8 |
| BRD3 | CATCACTGCAAACGTCACGT | 9 |
| C11orf65 | ATTCATGTGCGATTCAGATT | 10 |
| C11orf65 | TTAGCACAGAGATCTTCAAT | 11 |
| C11orf65 | TATCATCGTATAGAAAACAA | 12 |
| C1QL1 | CCGCGTCGTAGTTGTTGCCT | 13 |
| C1QL1 | CTTGATGAAGACCTCGTCGC | 14 |
| C1QL1 | CACGCGCGGCACCGTGGTGT | 15 |
| CLCC1 | TGGCGATTCGAAGATTCCTT | 16 |
| CLCC1 | GGATCCATATAATGTGTTAA | 17 |
| CLCC1 | TCTTTGTCTGCTCTGCATCG | 18 |
| CTSV | CGTGACGCCAGTGAAGAATC | 19 |
| CTSV | CATGTCACCAAAAGCATTCA | 20 |
| CTSV | ACAATGGCCATGAATGCTTT | 21 |
| CTSV | CATGAATCTTTCGCTCGTCC | 22 |
| CTSV | ACACAGAAGATTATATGGCG | 23 |
| EMC2 | CACAGAGTCAAGCGATTAAC | 24 |
| EMC2 | GATTGCCATTCGAAAAGCCC | 25 |
| EMC2 | TAATGAATATGCTTCTAAGC | 26 |
| EMC3 | GTCCTCCCTATGATTCTTAT | 27 |
| EMC3 | TCCGAAGCCCAAATACATTG | 28 |
| EMC3 | CATCCACCAATAAGAATCAT | 29 |
| EMC4 | TGCTTGTCCAAGTAACCGAC | 30 |
| EMC4 | AACCAATCCGATGCATGTGT | 31 |
| EMC4 | AGCTGTTGCCATGACGGCCC | 32 |
| EMC6 | ACGGCCGCCTCGCTGATGAA | 33 |
| EMC6 | GACCTCGGTGTCAGCGCTGT | 34 |
| EMC6 | AGACGTGCACCATGCCGTAG | 35 |
| FAM151B | GTATCATGCAGCTAACCACA | 36 |
| FAM151B | AGAACACAGCCAGCCAATTA | 37 |
| FAM151B | GCGCTTACCTGGGCCTCCAG | 38 |
| FAM210A | GTCCACGCCGCCACCCGTCA | 39 |
| FAM210A | GTGAAGTATCTGCGCAGTCA | 40 |
| FAM210A | AACCCAATGAGTTCTAGAAA | 41 |
| FBXL20 | ATTATACCTCAATATCCCTC | 42 |
| FBXL20 | TTACCGTAACAGGAGTTCTT | 43 |
| FBXL20 | TTCCCAAAGAACTCCTGTTA | 44 |
| GPR161 | CACAGTCGTCATCGTGGAGG | 45 |
| GPR161 | CACCTGCCATGAGCGCAGTG | 46 |
| GPR161 | AGAGACTCCACGTCCCGCTC | 47 |
| HSPA12B | ACGTGGAGACCGCTCTGCGC | 48 |
| HSPA12B | CACTGGGGACCGCTCCGGGC | 49 |
| HSPA12B | GGGCAATGCCGCAGCTTTCC | 50 |
| HSPA13 | CAAACCCACTGTTCACGCGA | 51 |
| HSPA13 | CCAAGTCTATCACCAAGACG | 52 |
| HSPA13 | GGCTGACGTCTTCCACGTCT | 53 |
| HSPA13 | AGTCGAGAGCCAACATATTC | 54 |
| HSPA13 | TACTGACAATGATGTATATG | 55 |
| MAN1B1 | AGCAACTGTCGAGATTGCAG | 56 |
| MAN1B1 | ATCCGCAGAGGACAGTCATC | 57 |
| MAN1B1 | CCTTCCGGGCGGGGATCCAC | 58 |
| MCCD1 | GCTCCAACAACTCTTCAGCT | 59 |
| MCCD1 | CTGCTCTTCCATGCTTGCTT | 60 |
| MCCD1 | AAACTTAGGCGCCTCCTCCA | 61 |
| MLL5 | TACAGCAGAGACGTCATACT | 62 |
| MLL5 | ATGCTCATGACGTTCGCCTC | 63 |
| MLL5 | GAGGACGAGCACCATAATTA | 64 |
| MLXIP | TGCCAAGTACCTCCGGCCGG | 65 |
| MLXIP | GGACCTCTCCAGCCTGGTCC | 66 |
| MLXIP | TGGCCCAATCCCCGGGAAAT | 67 |
| MMGT1 | GCATCATGGCGCCGTCGCTG | 68 |
| MMGT1 | CAGGCACTTACGCTGCGCAG | 69 |
| MMGT1 | CAAGGACATTTGATACGTTA | 70 |
| OMD | CCATTTAACATACATTCGTG | 71 |
| OMD | GCCAATATGAAACTTATCAG | 72 |
| OMD | CCTGTTTGGTAATCATCATC | 73 |
| OR52E2 | TATCCACAACTTCACACTTA | 74 |
| OR52E2 | TAGCGCCATCCTCACCAACA | 75 |
| OR52E2 | TTGAGTCGGGCTTCATGAGT | 76 |
| OST4 | CGCCATCTTCGCCAACATGC | 77 |
| OST4 | GAGCGACACGCCCAGCATGT | 78 |
| OST4 | CGTCAACAATCCCAAGAAGC | 79 |
| OSTC | TCAGTCATAGAACCGACACT | 80 |
| OSTC | CGAATCCAATGAACAGAAGA | 81 |
| OSTC | AGATTCCTTCTTCTGTTCAT | 82 |
| PARVG | CGTGAACCGGAGTCTGCAGC | 83 |
| PARVG | CTCCCTCCCAACCAACGTCC | 84 |
| PARVG | GTGGCAGGCCAAGTGGAGCG | 85 |
| PLA2G10 | AGTCCGGCTCACATAGGAAC | 86 |
| PLA2G10 | CTGCTGCTGCTGCTTCTACC | 87 |
| PLA2G10 | CCAGGATATTACGTGTGCAC | 88 |
| PVRL3 | GTGCTTCGTGCGCCGAACTC | 89 |
| PVRL3 | AACGGTCCCCGGAGCAAACC | 90 |
| PVRL3 | TCTGAAGCCAATAAACCATC | 91 |
| RAP1GAP | GAAGTTGGACGCGATCATGT | 92 |
| RAP1GAP | CGGATGCGACCCTCCCAGAC | 93 |
| RAP1GAP | TGCTGAATATGCCTGCTACA | 94 |
| RAP1GAP | TCCCGGACCCCGCTGTGTTC | 95 |
| RAP1GAP | GCTTTGTCAGCAAAAATTCC | 96 |
| RASA1 | TACCATCCGTCGTAAAACAA | 97 |
| RASA1 | AATGGTTGACAACATTCATC | 98 |
| RASA1 | CGATAGCAGAAGAACGCCTC | 99 |
| RASA1 | GCTTATAATTTACTAATGAC | 100 |
| RASA1 | ACAAGTACTCAATGACACAG | 101 |
| RBM25 | GCCATAATGCTCATTGGTAC | 102 |
| RBM25 | TTATTACATGACCTGCAAAT | 103 |
| SEC61B | TAGTGGCCCTGTTCCAGTAT | 104 |
| SEC61B | GTAGAATCGCCACATCCCCC | 105 |
| SEC61B | TCCTTACCTCTGCCGGACAG | 106 |
| SEC63 | GGTGTATGTGGTATCGTTTA | 107 |
| SEC63 | GTGATGAGGTTATGTTCATG | 108 |
| SEC63 | TTGGTATTCTCGGTCTGTTT | 109 |
| SEL1L | AGCATATCGGTATCTCCAAA | 110 |
| SEL1L | GCAGAAATGATGTATCAAAC | 111 |
| SEL1L | CTTGGCTTTCTGTATGCCTC | 112 |
| SERP1 | TCTTGGCGACGTTGCCGCGC | 113 |
| SERP1 | CGAAGATGGTCGCCAAGCAA | 114 |
| SERP1 | TCCTACAGACGCCTTCTCTT | 115 |
| SHC1 | CCTCCAGTCAATGCGTGCCC | 116 |
| SHC1 | TTACCAATGTAGCTCCCAAG | 117 |
| SHC1 | GGCAACATAGGCGACATACT | 118 |
| SHC1 | AGTCCAGGGCACGCATTGAC | 119 |
| SHC1 | CCCTTCATACCTGGACAGGG | 120 |
| SPCS1 | TGGCACTGCGCGTCAGTAGC | 121 |
| SPCS1 | ACGTGGCTGAACAGTTCGGG | 122 |
| SPCS1 | GAGAGGATGCCGGCGATAGA | 123 |
| SPCS3 | CTCTGAACCAAGTTGTCCTA | 124 |
| SPCS3 | TGTCCTGATCCTGTCACAAG | 125 |
| SPCS3 | ACCTAGAGAAAGAAGTGATC | 126 |
| SPCS3 | TCAAAACAATCTTGTCCCAT | 127 |
| SPCS3 | AAAAATGTAGAAGATTTCAC | 128 |
| SSR3 | CTATAACAACACTCTGTTCC | 129 |
| SSR3 | GACCCTAGTAAGCACATATT | 130 |
| SSR3 | TCTATTTGGAGCCAGTAGAC | 131 |
| SSR3 | CAGGGTTATACTGGCGAATA | 132 |
| SSR3 | AAGCAACAATGACCACGACC | 133 |
| STT3A | ACAGACATTCCGAATGTCGA | 134 |
| STT3A | AAGGTGGTACGTGACGATGG | 135 |
| STT3A | CTCGGTCATCAAACCAGTTA | 136 |
| SYVN1 | GTATGCCATCCTGATGACGA | 137 |
| SYVN1 | CCGCCATCATCACTGCCGTG | 138 |
| SYVN1 | GGCCAGGGCAATGTTCCGCA | 139 |
| TMEM100 | TGTTTGACTCTCCCGTCTCT | 140 |
| TMEM100 | GGTGATCACAACTTCACTCT | 141 |
| TMEM100 | TGTCTTCATCGCCGGCATCG | 142 |
| ZIC2 | ACACGCACCCCAGCTCGCTG | 143 |
| ZIC2 | GCTTCGCCAACAGCAGCGAC | 144 |
| ZIC2 | CTATGAGTCGTCCACGCCCC | 145 |
| ZNF488 | CTTTCGCCTAACGTCCGACC | 146 |
| ZNF488 | ACACTACAGACCTCGCTTGT | 147 |
| ZNF488 | AAAGTCGACCCCAACAAGCG | 148 |
| sgRNA control | CGCTTCCGCGGCCCGTTCAA | 149 |
| sgRNA control | ATCGTTTCCGCTTAACGGCG | 150 |

What is claimed is:

1. A method to inhibit flaviviral infection, the method comprising contacting a cell with a composition comprising a compound that downregulates or inhibits the ER signal peptidase complex components SPCS1, SPCS2 and/or SPCS3.

2. The method of claim 1, wherein the composition comprises a compound that downregulates or inhibits SPCS1.

3. The method of claim 1, wherein the flaviviral infection is due to a flavivirus selected from the group consisting of West Nile virus, Dengue virus, Japanese encephalitis virus or yellow fever virus.

4. The method of claim 1, wherein the amount of virus is reduced by a factor of at least 50.

5. The method of claim 1, wherein the amount of virus is reduced by a factor of at least 1,000.

6. The method of claim 1, wherein the amount of virus is reduced by a factor of at least 10,000.

7. A method to prevent flaviviral infection in a subject, the method comprising administering to the subject a composition comprising a compound that downregulates or inhibits the ER signal peptidase complex components SPCS1, SPCS2 and/or SPCS3.

8. The method of claim 7, wherein the composition comprises a compound that downregulates or inhibits SPCS1.

9. The method of claim 7, wherein the flaviviral infection is due to a flavivirus selected from the group consisting of West Nile virus, Dengue virus, Japanese encephalitis virus or yellow fever virus.

10. The method of claim 7, wherein the amount of virus is reduced by a factor of at least 50.

11. The method of claim 7, wherein the amount of virus is reduced by a factor of at least 1,000.

12. The method of claim 7, wherein the amount of virus is reduced by a factor of at least 10,000.

13. The method of claim 7, wherein the subject is protected from flaviviral infection.

14. A method to reduce the amount of flavivirus in a subject infected with a flavivirus, the method comprising administering to the subject a composition comprising a compound that downregulates or inhibits the ER signal peptidase complex components SPCS1, SPCS2 and/or SPCS3.

15. The method of claim 14, wherein the composition comprises a compound that downregulates or inhibits SPCS1.

16. The method of claim 14, wherein the flaviviral infection is due to a flavivirus selected from the group consisting of West Nile virus, Dengue virus, Japanese encephalitis virus or yellow fever virus.

17. The method of claim 14, wherein the amount of virus is reduced by a factor of at least 50.

18. The method of claim 14, wherein the amount of virus is reduced by a factor of at least 1,000.

19. The method of claim 14, wherein the amount of virus is reduced by a factor of at least 10,000.
